(19) United States
(12) Patent Application Publication
(10) Pub. No.: US 2006/0183746 A1
(43) Pub. Date: Aug. 17, 2006

(54) Certain imidazo[1,2-a]pyrazin-8-ylamines and method of inhibition of Bruton's tyrosine kinase by such compounds

(76) Inventors: Kevin S. Currie, North Branford, CT (US); Robert W. Desimone, Durham, CT (US); Scott A. Mitchell, East Haven, CT (US); Douglas A. I. Pippin, Chester Springs, PA (US); James W. Darrow, Wallingford, CT (US); Xiaobing Qian, Guilford, CT (US); Mark Velleca, New Haven, CT (US); Dapeng Qian, Guilford, CT (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/292,300

(22) Filed: Dec. 2, 2005

Related U.S. Application Data
(63) Continuation-in-part of application No. 10/861,791, filed on Jun. 4, 2004.
(60) Provisional application No. 60/475,634, filed on Jun. 4, 2003.
(60) Provisional application No. 60/519,311, filed on Nov. 11, 2003.
(60) Provisional application No. 60/633,378, filed on Dec. 6, 2004.

(51) Int. Cl. A61K 31/498; C07D 487/04

(52) U.S. Cl. 514/249; 544/330

(57) ABSTRACT

Certain compounds, such as at least one chemical entity choen from compounds of Formula 1 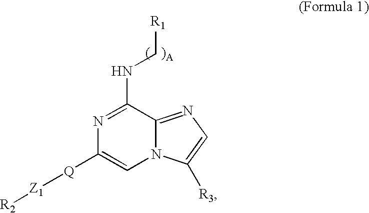 and pharmaceutically acceptable salts, solvates, crystal forms, chelates, non-covalent complexes, prodrugs, and mixtures thereof, may in certain embodiments be used to treat patients suffering from one or more diseases responsive to inhibition of tyrosine kinase activity. The diseases may, for instance, be responsive to inhibition of Btk activity and/or B-cell proliferation. Example diseases include cancer, an autoimmune and/or inflammatory disease, and an acute inflammatory reaction.

This application is a continuation-in-part of co-pending application Ser. No. 10/861,791, filed on Jun. 4, 2004, which claims priority to U.S. provisional application No. 60/475,634, filed on Jun. 4, 2003, and to U.S. provisional application No. 60/519,311, filed on Nov. 11, 2003. This application also claims priority to U.S. provisional application No. 60/633,378, filed on Dec. 6, 2004.

Provided herein are certain imidazo[1,2-a]pyrazinylamines and related compounds, compositions comprising such compounds, and methods of their use.

Protein kinases, the largest family of human enzymes, encompass well over 500 proteins. Bruton's Tyrosine Kinase (Btk) is a member of the Tec family of tyrosine kinases, and is a regulator of early B-cell development as well as mature B-cell activation, signaling and survival.

B-cell signaling through the B-cell receptor (BCR) leads to a wide range of biological outputs, which in turn depend on the developmental stage of the B-cell. The magnitude and duration of BCR signals must be precisely regulated. Aberrant BCR-mediated signaling can cause disregulated B-cell proliferation and/or the formation of pathogenic auto-antibodies leading to multiple autoimmune and/or inflammatory diseases. Mutation of Btk in humans results in X-linked agammaglobulinaemia (XLA). This disease is associated with the impaired maturation of B-cells, diminished immunoglobulin production, compromised T-cell-independent immune responses and marked attenuation of the sustained calcium sign upon BCR stimulation.

Evidence for the role of Btk in autoimmune and/or inflammatory disease has been established in Btk-deficient mouse models. For example, in standard murine preclinical models of systemic lupus erythematosus (SLE), Btk deficiency has been shown to result in a marked amelioration of disease progression. Moreover, Btk deficient mice are also resistant to developing collagen-induced arthritis and are less susceptible to Staphylococcus-induced arthritis.

A large body of evidence supports the role of B-cells and the humoral immune system in the pathogenesis of autoimmune and/or inflammatory diseases. Protein-based therapeutics (such as Rituxan) developed to deplete B-cells, represent an important approach to the treatment of a number of autoimmune and/or inflammatory diseases. Because of Btk's role in B-cell activation, inhibitors of Btk can be useful as inhibitors of B-cell mediated pathogenic activity (such as autoantibody production).

Btk is also expressed in mast cells and monocytes and has been shown to be important for the function of these cells. For example, Btk deficiency in mice is associated with impaired IgE-mediated mast cell activation (marked diminution of TNF-alpha and other inflammatory cytokine release), and greatly reduced TNF-alpha production by activated monocytes.

Thus, inhibition of Btk activity can be useful for the treatment of autoimmune and/or inflammatory diseases such as: SLE, rheumatoid arthritis, multiple vasculitides, idiopathic thrombocytopenic purpura (ITP), myasthenia gravis, and asthma. In addition, Btk has been reported to play a role in apoptosis, thus inhibition of Btk activity can be useful for the treatment of B-cell lymphoma and leukemia.

Provided is at least one chemical entity chosen from compounds of Formula 1 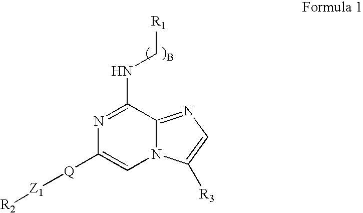
and pharmaceutically acceptable salts, solvates, crystal forms, chelates, non-covalent complexes, prodrugs, and mixtures thereof,
wherein
B is chosen from 0 and 1;
R₁ is chosen from
phenyl,
phenyl substituted with a group chosen from
   hydroxy,
   —CHO,
   —COOH,
   —CONH₂,
   —CONHOH,
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆ alkyl,
   substituted C₁-C₆ alkyl chosen from mono-, di-, and tri-substituted C₁-C₆ alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
   C₁-C₆ alkoxy,
   substituted C₁-C₆ alkoxy chosen from mono-, di-, and tri-substituted C₁-C₆ alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl,
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkyleneNR₁₀(SO₂)R₁₁,
   substituted —C₁-C₆alkyleneNR₁₀(SO₂)R₁₁ chosen from mono-, di-, and tri-substituted —C₁-C₆alkyleneNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyleneNR₁₀(C═O)R₁₁,
   substituted —C₀-C₆alkyleneNR₁₀(C═O)R₁₁ chosen from mono-, di-, and tri-substituted —C₀-C₆alkyleneNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylene(SO₂)R₁₀,
   substituted —C₀-C₆alkylene(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyleneNR₁₀(C═O)NR₁₁R₁₂,
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkyl amino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
heteroaryl substituted with a group chosen from
   hydroxy,
   —CHO,
   —COOH,
   —CONH₂,
   —CONHOH,
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl,
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂,
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl;
R₂ is chosen from
   C₁-C₇ alkyl,
   (C₁-C₆alkoxy)C₁-C₆alkoxy,
   (heterocycloalkyl)C₀-C₂alkyl,
   (C₃-C₇cycloalkyl)C₀-C₂alkyl,
   (phenyl)C₀-C₂alkyl,
   substituted (phenyl)C₀-C₂alkyl chosen from mono-, di-, and tri-substituted (phenyl)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
   (phenoxy)C₀-C₂alkyl,
   substituted (phenoxy)C₀-C₂alkyl chosen from mono-, di-, and tri-substituted (phenoxy)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
   (heteroaryl)C₀-C₂alkyl, and
   substituted (heteroaryl)C₀-C₂alkyl chosen from mono-, di-, and tri-substituted (heteroaryl)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl;
Z₁ is chosen from 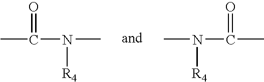
   wherein
   R₄ is chosen from
   hydrogen,
   C₁-C₆alkyl,
   C₁-C₆alkyl substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl;
   C₃-C₇cycloalkyl,
   C₃-C₇cycloalkyl substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl;
   heterocycloalkyl,
   heterocycloalkyl substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl;
   phenyl,
   phenyl substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl;
   heteroaryl, and
   heteroaryl substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl;
Q is chosen from phenylene and pyridylidene; and
R₃ is chosen from
   hydrogen,
   halo,
   C₁-C₇ alkyl,
   heterocycloalkyl,
   substituted heterocycloalkyl chosen from mono-, di-, and tri-substituted heterocycloalkyl wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
   C₃-C₇cycloalkyl,
   substituted C₃-C₇cycloalkyl chosen from mono-, di-, and tri-substituted C₃-C₇cycloalkyl wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
   heteroaryl, and
   substituted heteroaryl chosen from mono-, di-, and tri-substituted heteroaryl wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl.

In some embodiments, certain chemical entities described above exhibit an IC₅₀ of 1 micromolar or less, 100 nanomolar or less, or 10 nanomolar or less in standard biochemical assay for Btk activity. Certain chemical entities described herein exhibit an IC₅₀ value less than or equal to 10 micromolar, or an IC₅₀ value less than or equal to 1 micromolar, or an IC₅₀ value less than or equal to 500 nM. Certain chemical entities described herein are specific inhibitors of B-cell proliferation, exhibiting an IC₅₀ value that is at least 3-fold, for instance 5-fold, for instance 10-fold greater for T-cell proliferation than the IC₅₀ for B-cell proliferation.

Further embodiments provide methods for determining the presence of Btk in a sample, comprising contacting the sample with at least one chemical entity provided herein under conditions that permit detection of Btk activity, detecting a level of Btk activity in the sample, and therefrom determining the presence or absence of Btk in the sample.

Further embodiments provide pharmaceutical compositions comprising at least one chemical entity provided herein, together with at least one pharmaceutically acceptable vehicle chosen from carriers, adjuvants, and excipients.

Further embodiments provide packaged pharmaceutical compositions which comprise a pharmaceutical composition, comprising at least one chemical entity provided herein together with at least one pharmaceutically acceptable vehicle chosen from carriers, adjuvants, and excipients in a container and with instructions for using the pharmaceutical composition to treat a patient. The instructions may relate, for example, to using the pharmaceutical composition to treat a patient suffering from a disease responsive to inhibition of Btk activity.

Further embodiments provide methods of inhibiting Btk kinase, which comprises contacting a cell or cells expressing Btk, either in vivo or in vitro, with at least one chemical entity provided herein in an amount sufficient to detectably inhibit Btk activity in vitro.

Inhibiting Btk activity can inhibit B-cell proliferation. Thus further embodiments provide methods of inhibiting B-cell proliferation, which comprise contacting cells expressing Btk, either in vivo or in vitro with at least one chemical entity provided herein in an amount sufficient to detectably inhibit the activity of Btk in vitro.

Further embodiments provide methods of treating a mammal suffering from at least one disease responsive to inhibition of Btk activity, which comprise administering to the mammal an effective amount of at least one chemical entity provided herein, which is a highly active Btk inhibitor in an in vitro assay of Btk activity. In some embodiments, the at least one chemical entity is also a specific inhibitor of B-cell proliferation, exhibiting an IC₅₀ value that is at least 3-fold, for instance 5-fold, or for instance 10-fold greater for T-cell proliferation than the IC₅₀ value for B-cell proliferation.

Further embodiments provide methods for treating a patient having a disease responsive to inhibition of Btk activity and/or responsive to inhibition of B-cell proliferation. Such methods comprise administering to the patient an effective amount of at least one chemical entity provided herein. The patient may be a mammal, for example. In some embodiments, the patient is a human patient, however, methods of treating non-human patients are also included herein. For example, in some embodiments, the patient is a companion animal, such as a cat or dog, or the patient is a livestock animal, such as a horse, cow, or pig. Included herein are methods in which the disease responsive to Btk inhibition is chosen from cancers, autoimmune diseases, inflammatory diseases, and acute inflammatory reactions.

Further embodiments provide methods of treatment that include administering at least one chemical entity described above as a single active agent, and methods of treatment that include administering at least one chemical entity described above in combination with one or more other active agents.

As used in the present specification, the following words and phrases are generally intended to have the meanings as set forth below, except to the extent that the context in which they are used indicates otherwise. The following abbreviations and terms have the indicated meanings throughout:

As used herein, when any variable occurs more than one time in a chemical formula, its definition on each occurrence is independent of its definition at every other occurrence. In accordance with the usual meaning of “a” and “the” in patents, reference, for example, to “a” kinase or “the” kinase is inclusive of one or more kinases.

A dash (“-”) that is not between two letters or symbols is used to indicate a point of attachment for a substituent. For example, —CONH₂ is attached through the carbon atom.

“Alkyl” encompasses straight chain and branched chain having the indicated number of carbon atoms. For example C₁-C₆alkyl encompasses both straight and branched chain alkyl of from 1 to 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. For example, C₀ alkylene indicates a covalent bond and C₁ alkylene is a methylene group. When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl. “Lower alkyl” refers to alkyl groups having one to four carbons.

“Alkenyl” as used herein, indicates a straight or branched hydrocarbon chain comprising one or more unsaturated carbon-carbon bonds, which may occur in any stable point along the chain. Alkenyl groups described herein can have, for instance, from 2 to 12 carbons atoms. Examples of alkenyl groups include lower alkenyl groups, those alkenyl groups having from 2 to 8 carbon atoms, e.g. C₂-C₈, C₂-C₆, and C₂-C₄ alkenyl groups. Examples of alkenyl groups further include ethenyl, propenyl, and butenyl groups.

“Alkynyl” as used herein, indicates a straight or branched hydrocarbon chain comprising one or more triple carbon-carbon bonds that may occur in any stable point along the chain, such as ethynyl and propynyl. Alkynyl groups described herein may have, for instance, from 2 to 12 carbons atoms. Examples of alkynyl groups include lower alkynyl groups; those alkynyl groups having from 2 to 8 carbon atoms, e.g. C₂-C₈, C₂-C₆, and C₂-C₄ alkynyl groups.

“Cycloalkyl” indicates a monocyclic or multicyclic saturated hydrocarbon ring group, having the specified number of carbon atoms, for example from 3 to 10 ring carbon atoms. Monocyclic cycloalkyl groups can have, for example, from 3 to 8 carbon ring atoms or from 3 to 7 carbon ring atoms. Multicyclic cycloalkyl groups may have 2 or 3 fused cycloalkyl rings or contain bridged or caged cycloalkyl groups. Cycloalkyl substituents may be pendant to the substituted nitrogen or carbon atom, or where a substituted carbon atom may have two substituents a cycloalkyl group may be attached as a spiro group. Examples of cycloalkyl groups include cyclopropyl, cyclobutyl, cyclopentyl, and cyclohexyl as well as bridged or caged saturated ring groups such as norbornane or adamantane.

“Mono- and di-alkylcarboxamido” encompasses a group of the formula —(C═O)NRₐR_{b} where Rₐ and R_{b} are independently chosen from hydrogen and alkyl groups of the indicated number of carbon atoms, provided that Rₐ and R_{b} are not both hydrogen.

By “alkylthio” is meant an alkyl group of the indicated number of carbon atoms attached through a sulfur bridge.

“Alkoxy” indicates an alkyl group as defined above with the indicated number of carbon atoms attached through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

By “alkokycarbonyl” is meant an ester group of the formula (alkoxy)(C═O)— attached through the carbonyl carbon wherein the alkoxy group has the indicated number of carbon atoms. Thus a C₁-C₆alkoxycarbonyl group is an alkoxy group having from 1 to 6 carbon atoms attached through its oxygen to a carbonyl linker.

By “alkanoyl” is meant an alkyl group as defined above, attached through a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

By “amido” is meant —NH(C═O)R, wherein the R group is chosen from hydrogen and C₁-C₇alkyl. Except when R is hydrogen, each R may be unsubstituted or substituted with one or more, such as one, two or three, substituents independently chosen from halo, C₁-C₆alkyl, C₁-C₆haloalkyl, C₁-C₆haloalkoxy, C₁-C₆alkoxy, mono-(C₁-C₆ alkyl)amino, and di-(C₁-C₆ alkyl)amino.

By “amino” is meant the group —NH₂.

“Mono- and di-(alkyl)amino” encompasses secondary and tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

“Mono- and di-(alkyl)aminoalkyl” encompasses mono- and di-(alkyl)amino as defined above linked to an alkyl group.

By “amino(alkyl)” is meant an amino group linked to an alkyl group having the indicated number of carbons. Similarly “hydroxyalkyl” is a hydroxy group linked to an alkyl group.

“Aryl” encompasses:
5- and 6-membered carbocyclic aromatic rings, for example, benzene;
bicyclic ring systems wherein at least one ring is carbocyclic and aromatic, for example, naphthalene, indane, and tetralin; and
tricyclic ring systems wherein at least one ring is carbocyclic and aromatic, for example, fluorene.
   For example, aryl includes 5- and 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined below.

The term “aryloxy” refers to the group —O-aryl.

The term “halo” includes fluoro, chloro, bromo, and iodo, and the term “halogen” includes fluorine, chlorine, bromine, and iodine.

“Haloalkyl” indicates alkyl as defined above having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge.

“Heteroaryl” encompasses:
5- to 7-membered aromatic, monocyclic rings containing one or more, for example, from 1 to 4, or in certain embodiments, from 1 to 3, heteroatoms chosen from N, O, and S, with the remaining ring atoms being carbon; and
bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring.
   For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, systems (as numbered from the linkage position assigned priority 1), such as 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridizinyl, triazolyl, quinolinyl, pyrazolyl, and 5,6,7,8-tetrahydroisoquinoline. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above.

In the term “heteroarylalkyl,” heteroaryl and alkyl are as defined herein, and the point of attachment is on the alkyl group. This term encompasses, but is not limited to, pyridylmethyl, thiophenylmethyl, and (pyrrolyl)1-ethyl.

By “heterocycloalkyl” is meant a single aliphatic ring containing at least 2 carbon atoms in addition to 1-3 heteroatoms independently selected from oxygen, sulfur, and nitrogen, as well as combinations comprising at least one of the foregoing heteroatoms. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperdyl, and 2,5-piperzinyl. Morpholinyl groups are also contemplated, including 2-morpholinyl and 3-morpholinyl (numbered wherein the oxygen is assigned priority 1).

The term “substituted”, as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

The terms “substituted” alkyl, cycloalkyl, aryl, heterocycloalkyl, and heteroaryl, unless otherwise expressly defined, refer respectively to alkyl, cycloalkyl, aryl, heterocycloalkyl, and heteroaryl wherein one or more (up to 5, such as up to 3) hydrogen atoms are replaced by a substituent independently chosen from:

—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},

where R^{a} is chosen from optionally substituted C₁-C₆ alkyl, optionally substituted aryl, and optionally substituted heteroaryl;

R^{b} is chosen from H, optionally substituted C₁-C₆ alkyl, optionally substituted aryl, and optionally substituted heteroaryl; and

R^{c} is chosen from hydrogen and optionally substituted C₁-C₄ alkyl;

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted alkoxy” refers to alkoxy wherein the alkyl constituent is substituted (i.e., —O-(substituted alkyl)) wherein “substituted alkyl” refers to alkyl wherein one or more (up to 5, such as up to 3) hydrogen atoms are replaced by a substituent independently chosen from:

—R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —CONR^{b}R^{c}, —NR^{c}COR^{b}, —NR^{c}CO₂R^{a}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},

where R^{a} is chosen from optionally substituted C₁-C₆ alkyl, optionally substituted aryl, and optionally substituted heteroaryl;

R^{b} is chosen from H, optionally substituted C₁-C₆ alkyl, optionally substituted aryl, and optionally substituted heteroaryl; and

R^{c} is chosen from hydrogen and optionally substituted C₁-C₄ alkyl;

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl). In some embodiments, a substituted alkoxy group is “polyalkoxy” or —O-(optionally substituted alkylene)-(optionally substituted alkoxy), and includes groups such as —OCH₂CH₂OCH₃, and residues of glycol ethers such as polyethyleneglycol, and —O(CH₂CH₂O)ₓCH₃, where x is an integer of from 2 to 20, such as 2 to 10, and for example, 2 to 5. Another substituted alkoxy group is hydroxyalkoxy or —OCH₂(CH₂)_{y}OH, where y is an integer of 1 to 10, such as 1 to 4.

Compounds of Formula 1 include, but are not limited to, optical isomers of compounds of Formula 1, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula 1 include Z- and E-forms (or cis- and trans-forms) of compounds with carbon-carbon double bonds. Where compounds of Formula 1 exists in various tautomeric forms, chemical entities of the present invention include all tautomeric forms of the compound.

Compounds of Formula 1 also include isotopic variants. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Chemical entities of the present invention include, but are not limited to compounds of Formula 1 and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures of such compounds. In certain embodiments, the compounds described herein are in the form of pharmaceutically acceptable salts. Hence, the terms “chemical entity” and “chemical entities” also encompass pharmaceutically acceptable salts, solvates, crystal forms, chelates, non-covalent complexes, prodrugs, and mixtures.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COOH where n is 0-4, and like salts. Similarly, pharmaceutically acceptable cations include, but are not limited to sodium, potassium, calcium, aluminum, lithium, and ammonium.

In addition, if the compound of Formula 1 is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As noted above, prodrugs also fall within the scope of chemical entities, for example ester or amide derivatives of the compounds of Formula 1. The term “prodrugs” includes any compounds that become compounds of Formula 1 when administered to a patient, e.g., upon metabolic processing of the prodrug. Examples of prodrugs include, but are not limited to, acetate, formate, and benzoate and like derivatives of functional groups (such as alcohol or amine groups) in the compounds of Formula 1.

The term “solvate” refers to the chemical entity formed by the interaction of a solvent and a compound. Suitable solvates are pharmaceutically acceptable solvates, such as hydrates, including monohydrates and hemi-hydrates.

The term “chelate” refers to the chemical entity formed by the coordination of a compound to a metal ion at two (or more) points.

The term “active agent” is used to indicate a chemical entity which has biological activity. In certain embodiments, an “active agent” is a compound having pharmaceutical utility. For example an active agent may be an anti-cancer therapeutic.

The term “therapeutically effective amount” of a chemical entity of this invention means an amount effective, when administered to a human or non-human patient, to provide a therapeutic benefit such as amelioration of symptoms, slowing of disease progression, or prevention of disease e.g., a therapeutically effective amount may be an amount sufficient to decrease the symptoms of a disease responsive to Btk inhibition. In some embodiments, a therapeutically effective amount is an amount sufficient to reduce cancer symptoms, the symptoms of an autoimmune and/or inflammatory disease, or the symptoms of an acute inflammatory reaction. In some embodiments a therapeutically effective amount is an amount sufficient to decrease the number of detectable cancerous cells in an organism, detectably slow, or stop the growth of a cancerous tumor. In some embodiments, a therapeutically effective amount is an amount sufficient to shrink a cancerous tumor. In certain circumstances a patient suffering from cancer may not present symptoms of being affected. In some embodiments, a therapeutically effective amount of a chemical entity is an amount sufficient to prevent a significant increase or significantly reduce the detectable level of cancerous cells or cancer markers in the patient's blood, serum, or tissues. In methods described herein for treating autoimmune and/or inflammatory diseases or acute inflammatory reactions, a therapeutically effective amount may also be an amount sufficient, when administered to a patient, to detectably slow progression of the disease, or prevent the patient to whom the chemical entity is given from presenting symptoms of the autoimmune and/or inflammatory disease, or acute inflammatory response. In certain methods described herein for treating autoimmune and/or inflammatory diseases or acute inflammatory reactions, a therapeutically effective amount may also be an amount sufficient to produce a detectable decrease in the amount of a marker protein or cell type in the patient's blood or serum. For example, in some embodiments a therapeutically effective amount is an amount of a chemical entity described herein sufficient to significantly decrease the number of B-cells. In another example, in some embodiments a therapeutically effective amount is an amount of a chemical entity described herein sufficient to decrease the level of anti-acetylcholine receptor antibody in a patient's blood with the disease myasthenia gravis.

The term “inhibition” indicates a significant decrease in the baseline activity of a biological activity or process. “Inhibition of Btk activity” refers to a decrease in Btk activity as a direct or indirect response to the presence of at least one chemical entity described herein, relative to the activity of Btk in the absence of the at least one chemical entity. The decrease in activity may be due to the direct interaction of the compound with Btk, or due to the interaction of the chemical entity(ies) described herein with one or more other factors that in turn affect Btk activity. For example, the presence of the chemical entity(ies) may decrease Btk activity by directly binding to the Btk, by causing (directly or indirectly) another factor to decrease Btk activity, or by (directly or indirectly) decreasing the amount of Btk present in the cell or organism.

Inhibition of Btk activity also refers to observable inhibition of Btk activity in a standard biochemical assay for Btk activity, such as the ATP hydrolysis assay described below. In some embodiments, the chemical entity described herein has an IC₅₀ value less than or equal to 1 micromolar. In some embodiments, the chemical entity has an IC₅₀ value less than or equal to less than 100 nanomolar. In some embodiments, the chemical entity has an IC₅₀ value less than or equal to 10 nanomolar.

“Inhibition of B-cell proliferation” refers to inhibition of proliferation of abnormal B-cells, such as cancerous B-cells, e.g. lymphoma B-cells and/or inhibition of normal, non-diseased B-cells. The term “inhibition of B-cell proliferation” indicates any significant decrease in the number of B-cells, either in vitro or in vivo. Thus an inhibition of B-cell proliferation in vitro would be any significant decrease in the number of B-cells in an in vitro sample contacted with at least one chemical entity described herein as compared to a matched sample not contacted with the chemical entity(ies).

Inhibition of B-cell proliferation also refers to observable inhibition of B-cell proliferation in a standard thymidine incorporation assay for B-cell proliferation, such as the assay described herein. In some embodiments, the chemical entity has an IC₅₀ value less than or equal to 10 micromolar. In some embodiments, the chemical entity has an IC₅₀ value less than or equal to less than 1 micromolar. In some embodiments, the chemical entity has an IC₅₀ value less than or equal to 500 nanomolar.

By “significant” is meant any detectable change that is statistically significant in a standard parametric test of statistical significance such as Student's T-test, where p<0.05.

A “disease responsive to Btk inhibition” is a disease in which inhibiting Btk kinase provides a therapeutic benefit such as an amelioration of symptoms, decrease in disease progression, prevention or delay of disease onset, or inhibition of aberrant activity of certain cell-types (monocytes, B-cells, and mast cells).

“Treatment or treating means any treatment of a disease in a patient, including:
a) preventing the disease, that is, causing the clinical symptoms of the disease not to develop;
b) inhibiting the disease;
c) slowing or arresting the development of clinical symptoms; and/or
d) relieving the disease, that is, causing the regression of clinical symptoms.

“Patient” refers to an animal, such as a mammal, for example a human, that has been or will be the object of treatment, observation or experiment. The methods of the invention can be useful in both human therapy and veterinary applications. In some embodiments, the patient is a mammal, and in some embodiments the patient is human.

In certain embodiments, the invention provides at least one chemical entity chosen from compounds of Formula 1: 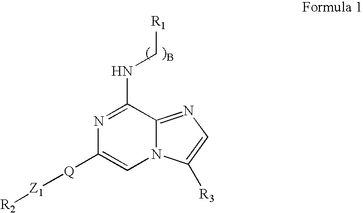
and pharmaceutically acceptable salts, solvates, crystal forms, chelates, non-covalent complexes, prodrugs, and mixtures thereof, wherein
B is chosen from 0 and 1;
R₁ is chosen from
   substituted phenyl wherein the substituent is chosen from
      hydroxy,
      —CHO,
      —COOH,
      —CONH₂,
      —CONHOH,
      C₂-C₆ alkenyl,
      substituted C₂-C₆ alkenyl chosen from mono-, di- and tri-substituted C₂-C₆ alkenyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₂-C₆ alkynyl,
      substituted C₂-C₆ alkynyl chosen from mono-, di- and tri-substituted C₂-C₆ alkynyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₁-C₆alkyl,
      substituted C₁-C₆alkyl chosen from mono-, di- and tri-substituted C₁-C₆alkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
      C₁-C₆alkoxy,
      substituted C₁-C₆alkoxy chosen from mono-, di- and tri-substituted C₁-C₆alkoxy, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
      heterocycloalkyl,
      substituted heterocycloalkyl chosen from mono-, di- and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di- and tri-substituted aryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      heteroaryl,
      substituted heteroaryl chosen from mono-, di- and tri-substituted heteroaryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₁-C₆alkylNR₁₀(SO₂)R₁₁,
      substituted —C₁-C₆alkylNR₁₀(SO₂)R₁₁, chosen from mono-, di- and tri-substituted —C₁-C₆alkylNR₁₀(SO₂)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkylNR₁₀(C═O)R₁₁,
      substituted —C₀-C₆alkylNR₁₀(C═O)R₁₁, chosen from mono-, di- and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkyl(SO₂)R₁₀,
      substituted —C₀-C₆alkyl(SO₂)R₁₀ chosen from mono-, di- and tri-substituted —C₀-C₆alkyl(SO₂)R₁₀, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkylNR₁₀(C═O)NR₁₁R₁₂,
      substituted —C₀-C₆alkylNR₁₀(C═O)NR₁₁R₁₂ chosen from mono-, di- and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      -L-G, where
         L is chosen from
         C₁-C₂alkylene,
         substituted C₁-C₂alkylene chosen from mono-, di- and tri-substituted C₁-C₂alkylene, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di- and tri-substituted C₀-C₂alkylene-O—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di- and tri-substituted —(C₁-C₂alkylene)(C═O)—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
         G is chosen from
         heterocycloalkyl,
         substituted heterocycloalkyl chosen from mono-, di- and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di- and tri-substituted C₃-C₇cycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di- and tri-substituted aryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
         heteroaryl, and
         substituted heteroaryl chosen from mono-, di- and tri-substituted heteroaryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   substituted heteroaryl wherein the substituent is chosen from
      hydroxy,
      —CHO,
      —COOH,
      —CONH₂,
      —CONHOH,
      C₂-C₆ alkenyl,
      substituted C₂-C₆ alkenyl chosen from mono-, di- and tri-substituted C₂-C₆ alkenyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₂-C₆ alkynyl,
      substituted C₂-C₆ alkynyl chosen from mono-, di- and tri-substituted C₂-C₆ alkynyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₁-C₆alkyl,
      substituted C₁-C₆alkyl chosen from mono-, di- and tri-substituted C₁-C₆alkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
      C₁-C₆alkoxy,
      substituted C₁-C₆alkoxy chosen from mono-, di- and tri-substituted C₁-C₆alkoxy, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
      heterocycloalkyl,
      substituted heterocycloalkyl chosen from mono-, di- and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      aryl,
      substituted aryl chosen from mono-, di- and tri-substituted aryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      heteroaryl,
      substituted heteroaryl chosen from mono-, di- and tri-substituted heteroaryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₁-C₆alkylNR₁₀(SO₂)R₁₁,
      substituted —C₁-C₆alkylNR₁₀(SO₂)R₁₁, chosen from mono-, di- and tri-substituted —C₁-C₆alkylNR₁₀(SO₂)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkylNR₁₀(C═O)R₁₁,
      substituted —C₀-C₆alkylNR₁₀(C═O)R₁₁ chosen from mono-, di- and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkyl(SO₂)R₁₀,
      substituted —C₀-C₆alkyl(SO₂)R₁₀ chosen from mono-, di- and tri-substituted —C₀-C₆alkyl(SO₂)R₁₀, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkylNR₁₀(C═O)NR₁₁R₁₂,
      substituted —C₀-C₆alkylNR₁₀(C═O)NR₁₁R₁₂ chosen from mono-, di- and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      -L-G, where
         L is chosen from
         C₁-C₂alkylene,
         substituted C₁-C₂alkylene chosen from mono-, di- and tri-substituted C₁-C₂alkylene, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di- and tri-substituted C₀-C₂alkylene-O—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di- and tri-substituted —(C₁-C₂alkylene)(C═O)—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
         G is chosen from
         heterocycloalkyl,
         substituted heterocycloalkyl chosen from mono-, di- and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di- and tri-substituted C₃-C₇cycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di- and tri-substituted aryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
         heteroaryl, and
         substituted heteroaryl chosen from mono-, di- and tri-substituted heteroaryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl, and
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl;
R₂ is chosen from
   C₁-C₇ alkyl,
   (C₁-C₆alkoxy)C₁-C₆alkoxy,
   (heterocycloalkyl)C₀-C₂alkyl,
   (C₃-C₇cycloalkyl)C₀-C₂alkyl,
   (phenyl)C₀-C₂alkyl,
   substituted (phenyl)C₀-C₂alkyl chosen from mono-, di-, and tri-substituted (phenyl)C₀-C₂alkyl, wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₁-C₂alkyl, C₁-C₃haloalkyl, heterocycloalkyl, phenyl, and heteroaryl,
   (phenoxy)C₀-C₂alkyl,
   substituted (phenoxy)C₀-C₂alkyl chosen from mono-, di-, and tri-substituted (phenoxy)C₀-C₂alkyl, wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
   (heteroaryl)C₀-C₂alkyl, and
   substituted (heteroaryl)C₀-C₂alkyl chosen from mono-, di-, and tri-substituted (heteroaryl)C₀-C₂alkyl, wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl;
Z₁ is chosen from 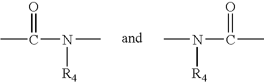
   wherein
   R₄ is chosen from
   hydrogen,
   C₁-C₆alkyl,
   substituted C₁-C₆alkyl chosen from C₁-C₆alkyl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
   C₃-C₇cycloalkyl,
   substituted C₃-C₇cycloalkyl chosen from C₃-C₇cycloalkyl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
   heterocycloalkyl,
   substituted heterocycloalkyl chosen from heterocycloalkyl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
   phenyl,
   substituted phenyl chosen from phenyl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
   heteroaryl, and
   substituted heteroaryl chosen from heteroaryl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl;
Q is chosen from phenylene and pyridylidene; and
R₃ is chosen from
   hydrogen,
   halo,
   C₁-C₇ alkyl,
   heterocycloalkyl,
   substituted heterocycloalkyl chosen from mono-, di-, and tri-substituted heterocycloalkyl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
   C₃-C₇cycloalkyl,
   substituted C₃-C₇cycloalkyl chosen from mono-, di-, and tri-substituted C₃-C₇cycloalkyl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
   heteroaryl, and
   substituted heteroaryl chosen from mono-, di-, and tri-substituted heteroaryl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl.

In some embodiments, A is 0. In some embodiments, A is 1.

In some embodiments, R₁ is chosen from
phenyl substituted with a substituent chosen from
   hydroxy,
   —CHO,
   —COOH,
   —CONH₂,
   —CONHOH,
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl,
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂,
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a substituent chosen from
   hydroxy,
   —CHO,
   —COOH,
   —CONH₂,
   —CONHOH,
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl,
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁, chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂,
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkyl amino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments, R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH,
wherein R₁ is optionally substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments, R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH, wherein
R₁ is optionally further mono-, di-, or tri-substituted with groups independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

In some embodiments, R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH, wherein
R₁ is optionally further mono-, di-, or tri-substituted with groups independently chosen from hydroxy, cyano, halo, C₁-C₂alkyl, and C₁-C₂alkoxy.

In some embodiments, R₁ is chosen from
phenyl substituted with a group chosen from
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkokycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl, and
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl, and
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments, R₁ is chosen from
phenyl substituted with a group chosen from
   C₁-C₆alkyl,
   substituted C₁-C₆alkyl chosen from mono-, di-, and tri-substituted C₁-C₆alkyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
   C₁-C₆alkoxy, and
   substituted C₁-C₆alkoxy chosen from mono-, di-, and tri-substituted C₁-C₆alkoxy, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido, and
pyridyl substituted with a group chosen from
   C₁-C₆alkyl,
   substituted C₁-C₆alkyl chosen from mono-, di-, and tri-substituted C₁-C₆alkyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
   C₁-C₆alkoxy, and
   substituted C₁-C₆alkoxy chosen from mono-, di-, and tri-substituted C₁-C₆alkoxy, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

In some embodiments, R₁ is chosen from
phenyl substituted with a group chosen from C₁-C₆hydroxyalkyl, C₁-C₆hydroxyalkoxy, (mono-C₁-C₆alkylamino)C₁-C₆alkoxy, (di-C₁-C₆alkylamino)C₁-C₆alkoxy, (C₁-C₆alkoxy)(C₁-C₆alkylamino)C₀-C₆alkyl, and (C₁-C₆alkoxy)(C₁-C₆alkoxy)C₁-C₆alkoxy, and
pyridyl substituted with a group chosen from C₁-C₆hydroxyalkyl, C₁-C₆hydroxyalkoxy, (mono-C₁-C₆alkylamino)C₁-C₆alkoxy, (di-C₁-C₆alkylamino)C₁-C₆alkoxy, (C₁-C₆alkoxy)(C₁-C₆alkylamino)C₀-C₆alkyl, and (C₁-C₆alkoxy)(C₁-C₆alkoxy)C₁-C₆alkoxy,
wherein R₁ is optionally further mono-, di-, or tri-substituted with substituents independently chosen from hydroxy, cyano, halo, C₁-C₂alkyl, and C₁-C₂alkoxy.

In some embodiments, R₁ is chosen from
phenyl substituted with a group chosen from
   pyrrolidinyl,
   substituted pyrrolidinyl chosen from mono-, di- and tri-substituted pyrrolidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di- and tri-substituted morpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di- and tri-substituted thiomorpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di- and tri-substituted piperazinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperidinyl,
   substituted piperidinyl chosen from mono-, di- and tri-substituted piperidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di- and tri-substituted phenyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di- and tri-substituted imidazolyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine,
   substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
   pyrrolidinyl,
   substituted pyrrolidinyl chosen from mono-, di- and tri-substituted pyrrolidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di- and tri-substituted morpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di- and tri-substituted thiomorpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di- and tri-substituted piperazinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperidinyl,
   substituted piperidinyl chosen from mono-, di- and tri-substituted piperidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di- and tri-substituted phenyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di- and tri-substituted imidazolyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine, and
   substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

In some embodiments, R₁ is chosen from
phenyl substituted with a group chosen from
   pyrrolidinyl,
   substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperidinyl,
   substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine, and
   substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
   pyrrolidinyl,
   substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperidinyl,
   substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkyl amino, di-C₁-C₄alkyl amino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine, and
   substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy.

In some embodiments, R₁ is chosen from
phenyl substituted with a group chosen from
   —C₁-C₆alkylene(C═O)OR₁₀,
   substituted —C₁-C₆alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylene(C═O)NR₁₀R₁₁,
   substituted —C₀-C₆alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁, chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkyl amino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂, and
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from
   —C₁-C₆alkylene(C═O)OR₁₀,
   substituted —C₁-C₆alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylene(C═O)NR₁₀R₁₁,
   substituted —C₀-C₆alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂, and
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl, and
wherein R₁₀, R₁₁, and R₁₂ are independently chosen from hydrogen, hydroxy, C₁-C₆alkyl, C₁-C₆alkoxy, C₃-C₇cycloalkyl, and heterocycloalkyl.

In some embodiments, R₁ is chosen from
phenyl substituted with a group chosen from
   —C₁-C₄alkylene(C═O)OR₁₀,
   substituted —C₁-C₄alkylene(C═O)OR₁₀ chosen from mono-, di-, and tri-substituted —C₁-C₄alkylene(C═O)OR₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylene(C═O)NR₁₀R₁₁,
   substituted —C₀-C₄alkylene(C═O)NR₁₀R₁₁ chosen from mono-, di-, and tri-substituted —C₀-C₄alkylene(C═O)NR₁₀R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₁-C₄alkylNR₁₀(SO₂)R₁₁,
   substituted —C₁-C₄alkylNR₁₀(SO₂)R₁₁ chosen from mono-, di-, and tri-substituted —C₁-C₄alkylNR₁₀(SO₂)R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylNR₁₀(C═O)R₁₁,
   substituted —C₀-C₄alkylNR₁₀(C═O)R₁₁ chosen from mono-, di-, and tri-substituted —C₀-C₄alkylNR₁₀(C═O)R₁₁, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkyl(SO₂)R₁₀,
   substituted —C₀-C₄alkyl(SO₂)R₁₀ chosen from mono-, di-, and tri-substituted —C₀-C₄alkyl(SO₂)R₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylNR₁₀(C═O)NR₁₁R₁₂, and
   substituted —C₀-C₄alkylNR₁₀(C═O)NR₁₁R₁₂ chosen from mono-, di-, and tri-substituted —C₀-C₄alkylNR₁₀(C═O)NR₁₁R₁₂, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
   —C₁-C₄alkylene(C═O)OR₁₀,
   substituted —C₁-C₄alkylene(C═O)OR₁₀ chosen from mono-, di-, and tri-substituted —C₁-C₄alkylene(C═O)OR₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylene(C═O)NR₁₀R₁₁,
   substituted —C₀-C₄alkylene(C═O)NR₁₀R₁₁ chosen from mono-, di-, and tri-substituted —C₀-C₄alkylene(C═O)NR₁₀R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₁-C₄alkylNR₁₀(SO₂)R₁₁,
   substituted —C₁-C₄alkylNR₁₀(SO₂)R₁₁ chosen from mono-, di-, and tri-substituted —C₁-C₄alkylNR₁₀(SO₂)R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylNR₁₀(C═O)R₁₁,
   substituted —C₀-C₄alkylNR₁₀(C═O)R₁₁, chosen from mono-, di-, and tri-substituted —C₀-C₄alkylNR₁₀(C═O)R₁₁, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkyl(SO₂)R₁₀,
   substituted —C₀-C₄alkyl(SO₂)R₁₀ chosen from mono-, di-, and tri-substituted —C₀-C₄alkyl(SO₂)R₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylNR₁₀(C═O)NR₁₁R₁₂ and
   substituted —C₀-C₄alkylNR₁₀(C═O)NR₁₁R₁₂ chosen from mono-, di-, and tri-substituted —C₀-C₄alkylNR₁₀(C═O)NR₁₁R₁₂, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy, and
wherein R₁₀, R₁₁, and R₁₂ are independently chosen from hydrogen, hydroxy, C₁-C₆alkyl, C₁-C₆alkoxy, C₃-C₁₀cycloalkyl, pyrrolidinyl, morpholinyl, thiomorpholinyl, piperazinyl, piperidinyl, and [1,4]diazepanyl.

In some embodiments, R₁ is chosen from
phenyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   G is chosen from
      heterocycloalkyl,
      substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₃-C₇cycloalkyl,
      substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      aryl,
      substituted aryl chosen from mono-, di-, and tri-substituted aryl wherein the substituents are independently-chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      heteroaryl, and
      substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   G is chosen from
      heterocycloalkyl,
      substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₃-C₇cycloalkyl,
      substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      aryl,
      substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      heteroaryl, and
      substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments, R₁ is chosen from phenyl substituted with -L-G, where
L is chosen from
   C₁-C₂alkylene,
   substituted C₁-C₂alkylene chosen from mono-, di-, and tri-substituted C₁-C₂alkylene wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   C₀-C₂alkylene-O—,
   substituted C₀-C₂alkylene-O— chosen from mono-, di-, and tri-substituted C₀-C₂alkylene-O— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —(C═O)—,
   —(C₁-C₂alkyl)(C═O)—, and
   substituted —(C₁-C₂alkyl)(C═O)— chosen from mono-, di-, and tri-substituted —(C₁-C₂alkyl)(C═O)— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   and
G is chosen from
   pyrrolidinyl,
   substituted pyrrolidinyl chosen from mono-, di-, and tri-substituted pyrrolidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di-, and tri-substituted morpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di-, and tri-substituted thiomorpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di-, and tri-substituted piperazinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, and di-C₁-C₄alkylcarboxamido,
   piperidinyl,
   substituted piperidinyl chosen from mono-, di-, and tri-substituted piperidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di-, and tri-substituted [1,4]diazepanyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di-, and tri-substituted phenyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di-, and tri-substituted imidazolyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine, and
   substituted 5,6-dihydro-8H-[1,2,4]triazolo[4,3-a]pyrazine chosen from mono-, di-, and tri-substituted 5,6-dihydro-8H-[1,2,4]triazolo[4,3-a]pyrazine wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
   G is chosen from
      pyrrolidinyl,
      substituted pyrrolidinyl chosen from mono-, di-, and tri-substituted pyrrolidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      morpholinyl,
      substituted morpholinyl chosen from mono-, di-, and tri-substituted morpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      thiomorpholinyl,
      substituted thiomorpholinyl chosen from mono-, di-, and tri-substituted thiomorpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperazinyl,
      substituted piperazinyl chosen from mono-, di-, and tri-substituted piperazinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperidinyl,
      substituted piperidinyl chosen from mono-, di-, and tri-substituted piperidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      [1,4]diazepanyl,
      substituted [1,4]diazepanyl chosen from mono-, di-, and tri-substituted [1,4]diazepanyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      octahydro-pyrido[1,2-a]pyrazin-2-yl,
      substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      phenyl,
      substituted phenyl chosen from mono-, di-, and tri-substituted phenyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      imidazolyl,
      substituted imidazolyl chosen from mono-, di-, and tri-substituted imidazolyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      5,6-dihydro-8H-[1,2,4]triazolo[4,3-a]pyrazine, and
      substituted 5,6-dihydro-8H-[1,2,4]triazolo[4,3-a]pyrazine chosen from mono-, di-, and tri-substituted 5,6-dihydro-8H-[1,2,4]triazolo[4,3-a]pyrazine wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

In some embodiments, R₁ is chosen from
phenyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
   G is chosen from
      pyrrolidinyl,
      substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      morpholinyl,
      substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      thiomorpholinyl,
      substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperazinyl,
      substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperidinyl,
      substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      [1,4]diazepanyl,
      substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      octahydro-pyrido[1,2-a]pyrazin-2-yl,
      substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      phenyl,
      substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      imidazolyl,
      substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      5,6-dihydro-8H-[1,2,4]triazolo[4,3-a]pyrazine, and
      substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
   G is chosen from
      pyrrolidinyl,
      substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      morpholinyl,
      substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      thiomorpholinyl,
      substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperazinyl,
      substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperidinyl,
      substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      [1,4]diazepanyl,
      substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      octahydro-pyrido[1,2-a]pyrazin-2-yl,
      substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      phenyl,
      substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      imidazolyl,
      substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      5,6-dihydro-8H-[1,2,4]triazolo[4,3-a]pyrazine, and
      substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy.

In some embodiments, L is chosen from
phenyl substituted with a group chosen from —CH₂—, —(C═O)—, and —(CH₂)(C═O)—, and
pyridyl substituted with a group chosen from —CH₂—, —(C═O)—, and —(CH₂)(C═O)—, and
G is chosen from
   pyrrolidinyl,
   substituted pyrrolidinyl chosen from mono-, di- and tri-substituted pyrrolidinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   morpholinyl,
   substituted morpholinyl chosen from mono-, di- and tri-substituted morpholinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   thiomorpholinyl,
   substituted thiomorpholinyl chosen from mono-, di- and tri-substituted thiomorpholinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   piperazinyl,
   substituted piperazinyl chosen from mono-, di- and tri-substituted piperazinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   piperidinyl,
   substituted piperidinyl chosen from mono-, di- and tri-substituted piperidinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkyl amino, mono-C₁-C₂alkylcarboxamido, and di-C₁-C₂alkylcarboxamido,
   octahydro-pyrido[1,2-a]pyrazin-2-yl, and
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
wherein R₁ is optionally further mono-, di-, or tri-substituted with substituents independently chosen from hydroxy, halo, C₁-C₂alkyl, and C₁-C₂alkoxy.

In some embodiments, R₂ is chosen from
isoxazol-5-yl,
substituted isoxazol-5-yl chosen from mono-, di-, and tri-substituted isoxazol-5-yl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkyl carboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl;
thiazol-4-yl,
substituted thiazol-4-yl chosen from mono-, di-, and tri-substituted thiazol-4-yl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl;
[1,2,3]-thiadiazol-4-yl, and
substituted [1,2,3]-thiadiazol-4-yl chosen from mono-, di-, and tri-substituted [1,2,3]-thiadiazol-4-yl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl.

In some embodiments, R₂ is chosen from
isoxazol-5-yl,
substituted isoxazol-5-yl chosen from mono-, di-, and tri-substituted isoxazol-5-yl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl,
thiazol-4-yl,
substituted thiazol-4-yl chosen from mono-, di-, and tri-substituted thiazol-4-yl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl,
[1,2,3]-thiadiazol-4-yl, and
substituted [1,2,3]-thiadiazol-4-yl chosen from mono-, di-, and tri-substituted [1,2,3]-thiadiazol-4-yl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl.

Also provided is at least one chemical entity chosen from compounds of Formula 2 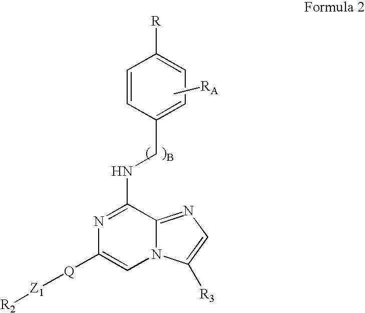
and pharmaceutically acceptable salts, solvates, crystal forms, chelates, non-covalent complexes, prodrugs, and mixtures thereof, wherein B, Q, Z₁, R₂, and R₃ are as described for compounds of Formula 1, and further wherein R is chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is chosen from
   C₁-C₂alkylene,
   substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₀-C₂alkylene-O—,
   substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —(C═O)—,
   —(C₁-C₂alkylene)(C═O)—, and
   substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
   heterocycloalkyl,
   substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₃-C₇cycloalkyl,
   substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl, and
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl, and
wherein R_{A} is optional and when present, can be present up to four times, and is independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

Also provided is at least one chemical entity chosen from compounds of Formula 3 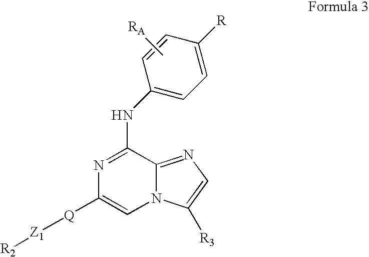
and pharmaceutically acceptable salts, solvates, crystal forms, chelates, non-covalent complexes, prodrugs, and mixtures thereof, wherein R and R_{A} are as described for compounds of Formula 2 and Q, Z₁, R₂, and R₃ are as described for compounds of Formula 1.

In some embodiments, R₂ is chosen from
(phenyl)C₀-C₂alkyl,
substituted (phenyl)C₀-C₂alkyl chosen from mono-, di-, and tri-substituted (phenyl)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
(phenoxy)C₀-C₂alkyl,
substituted (phenoxy)C₀-C₂alkyl chosen from mono-, di-, and tri-substituted (phenoxy)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
(pyridyl)C₀-C₂alkyl, and
substituted (pyridyl)C₀-C₂alkyl chosen from mono-, di-, and tri-substituted (pyridyl)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl.

In some embodiments, R₂ is chosen from
(phenyl)C₀-C₂alkyl,
substituted (phenyl)C₀-C₂alkyl chosen from mono-, di-, and tri-substituted (phenyl)C₀-C₂alkyl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl,
(phenoxy)C₀-C₂alkyl,
substituted (phenoxy)C₀-C₂alkyl chosen from mono-, di-, and tri-substituted (phenoxy)C₀-C₂alkyl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl,
(pyridyl)C₀-C₂alkyl, and
substituted (pyridyl)C₀-C₂alkyl chosen from mono-, di-, and tri-substituted (pyridyl)C₀-C₂alkyl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl.

In some embodiments, Z₁ is chosen from 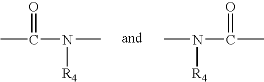
and
R₄ is chosen from hydrogen, C₁-C₆alkyl, and C₃-C₇cycloalkyl. In some embodiments, R₄ is chosen from hydrogen and methyl.

Also provided is at least one chemical entity chosen from compounds of Formula 4 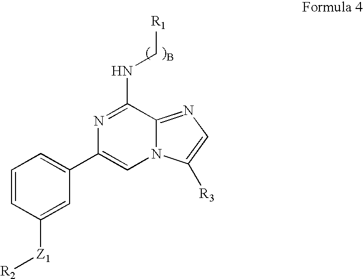
and pharmaceutically acceptable salts, solvates, crystal forms, chelates, non-covalent complexes, prodrugs, and mixtures thereof, wherein R₁, B, Z₁, R₂, and R₃ are as described for compounds of Formula 1.

Also provided is at least one chemical entity chosen from compounds of Formula 5 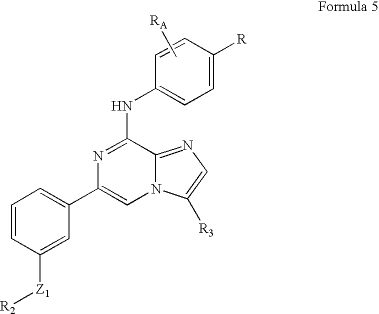
and pharmaceutically acceptable salts, solvates, crystal forms, chelates, non-covalent complexes, prodrugs, and mixtures thereof wherein R₁, Z₁, R₂, and R₃ are as described for compounds of Formula 1 and further wherein R is chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C f-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁, chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is
   C₁-C₂alkyl,
   substituted C₁-C₂alkyl chosen from mono-, di-, and tri-substituted C₁-C₂alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₀-C₂alkylene-O—,
   substituted C₀ C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —(C═O)—,
   —(C₁-C₂alkyl)(C═O)—, and
   substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is
   heterocycloalkyl,
   substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₃-C₇cycloalkyl,
   substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl, and
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R_{A} is optional and when present, can be present up to four times, and is independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl, and
wherein R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl.

In some embodiments, R₃ is chosen from hydrogen and methyl

Also provided is at least one chemical entity chosen from compounds of Formula 6 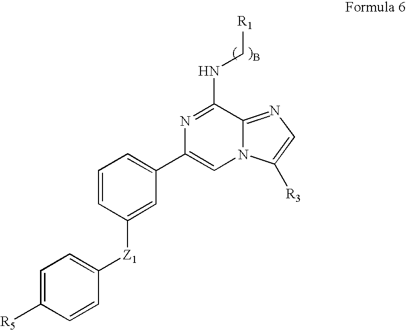
and pharmaceutically acceptable salts, solvates, crystal forms, chelates, non-covalent complexes, prodrugs, and mixtures thereof, wherein
B is chosen from 0 and 1;
R₁ is chosen from
   phenyl,
   phenyl substituted with a group chosen from
      hydroxy,
      —CHO,
      —COOH,
      —CONH₂,
      —CONHOH,
      C₂-C₆ alkenyl,
      substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₂-C₆ alkynyl,
      substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₁-C₆alkyl,
      substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
      C₁-C₆alkoxy,
      substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      heterocycloalkyl,
      substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      aryl,
      substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      heteroaryl,
      substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₁-C₆alkylNR₁₀(SO₂)R₁₁,
      substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkylNR₁₀(C═O)R₁₁,
      substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkyl(SO₂)R₁₀,
      substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkylNR₁₀(C═O)NR₁₁R₁₂,
      substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl, and
   heteroaryl substituted with a group chosen from
      hydroxy,
      —CHO,
      —COOH,
      —CONH₂,
      —CONHOH,
      C₂-C₆ alkenyl,
      substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₂-C₆ alkynyl,
      substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      C₁-C₆alkyl,
      substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
      C₁-C₆alkoxy,
      substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      heterocycloalkyl,
      substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, di-C₁-C₄alkylcarboxamido, and phenyl,
      aryl,
      substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      heteroaryl,
      substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₁-C₆alkylNR₁₀(SO₂)R₁₁,
      substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkylNR₁₀(C═O)R₁₁,
      substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkyl(SO₂)R₁₀,
      substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
      —C₀-C₆alkylNR₁₀(C═O)NR₁₁R₁₂ and
      substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O-chosen from mono-, di-, and tri-substituted C₀-C₂alkylene-O-wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl, and
wherein R₁ is further optionally substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl;
R₃ is chosen from
   hydrogen,
   halo,
   C₁-C₇ alkyl,
   heterocycloalkyl,
   substituted heterocycloalkyl chosen from mono-, di-, and tri-substituted heterocycloalkyl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
   C₃-C₇cycloalkyl,
   substituted C₃-C₇cycloalkyl chosen from mono-, di-, and tri-substituted C₃-C₇cycloalkyl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
   heteroaryl, and
   substituted heteroaryl chosen from mono-, di-, and tri-substituted heteroaryl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl;
Z₁ is chosen from 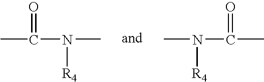
   wherein
   R₄ is chosen from
   hydrogen,
   C₁-C₆alkyl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
   C₃-C₇cycloalkyl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
   heterocycloalkyl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
   phenyl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl, and
   heteroaryl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl; and
   R₅ is chosen from isopropyl and t-butyl.

In some embodiments, A is 0.

In some embodiments, A is 1.

In some embodiments,
R₁ is chosen from
phenyl,
phenyl substituted with a group chosen from
   hydroxy,
   —CHO,
   —COOH,
   —CONH₂,
   —CONHOH,
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl,
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂,
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
pyridyl, and
pyridyl substituted with
   hydroxy,
   —CHO,
   —COOH,
   —CONH₂,
   —CONHOH,
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl,
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂, and
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments,
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments,
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

In some embodiments,
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH,
wherein R₁ is optionally further mono-, di-, or tri-substituted with substituents independently chosen from hydroxy, cyano, halo, C₁-C₂alkyl, and C₁-C₂alkoxy.

In some embodiments,
R₁ is chosen from
phenyl substituted with a group chosen from
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl, and
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from,
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl, and
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments,
R₁ is chosen from
   phenyl substituted with a group chosen from
      C₁-C₆alkyl,
      substituted C₁-C₆alkyl chosen from mono-, di-, and tri-substituted C₁-C₆alkyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
      C₁-C₆alkoxy, and
      substituted C₁-C₆alkoxy chosen from mono-, di-, and tri-substituted C₁-C₆alkoxy, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido, and
   pyridyl substituted with a group chosen from
      C₁-C₆alkyl,
      substituted C₁-C₆alkyl chosen from mono-, di-, and tri-substituted C₁-C₆alkyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
      C₁-C₆alkoxy, and
      substituted C₁-C₆alkoxy chosen from mono-, di-, and tri-substituted C₁-C₆alkoxy, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

In some embodiments,
R₁ is chosen from
phenyl substituted with a group chosen from C₁-C₆hydroxyalkyl, C₁-C₆hydroxyalkoxy, (mono-C₁-C₆alkylamino)C₁-C₆alkoxy, (di-C₁-C₆alkylamino)C₁-C₆alkoxy, (C₁-C₆alkoxy)(C₁-C₆alkylamino)C₀-C₆alkyl, and (C₁-C₆alkoxy)(C₁-C₆alkoxy)C₁-C₆alkoxy, and
pyridyl, substituted with a group chosen from C₁-C₆hydroxyalkyl, C₁-C₆hydroxyalkoxy, (mono-C₁-C₆alkylamino)C₁-C₆alkoxy, (di-C₁-C₆alkylamino)C₁-C₆alkoxy, (C₁-C₆alkoxy)(C₁-C₆alkylamino)C₀-C₆alkyl, and (C₁-C₆alkoxy)(C₁-C₆alkoxy)C₁-C₆alkoxy,
wherein R₁ is optionally further mono-, di-, or tri-substituted with substituents independently chosen from hydroxy, cyano, halo, C₁-C₂alkyl, and C₁-C₂alkoxy.

In some embodiments,
R₁ is chosen from
phenyl substituted with a group chosen from
   pyrrolidinyl,
   substituted pyrrolidinyl chosen from mono-, di- and tri-substituted pyrrolidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di- and tri-substituted morpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di- and tri-substituted thiomorpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di- and tri-substituted piperazinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperidinyl,
   substituted piperidinyl chosen from mono-, di- and tri-substituted piperidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di- and tri-substituted phenyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di- and tri-substituted imidazolyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine, and
   substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
   pyrrolidinyl,
   substituted pyrrolidinyl chosen from mono-, di- and tri-substituted pyrrolidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di- and tri-substituted morpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di- and tri-substituted thiomorpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di- and tri-substituted piperazinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperidinyl,
   substituted piperidinyl chosen from mono-, di- and tri-substituted piperidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di- and tri-substituted phenyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di- and tri-substituted imidazolyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine, and
   substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

In some embodiments,
R₁ is chosen from
phenyl substituted with a group chosen from
   pyrrolidinyl,
   substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperidinyl,
   substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine, and
   substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
   pyrrolidinyl,
   substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   morpholinyl,
   substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   thiomorpholinyl,
   substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperazinyl,
   substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   piperidinyl,
   substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   octahydro-pyrido[1,2-a]pyrazin-2-yl,
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   phenyl,
   substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   imidazolyl,
   substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   5,6-dihydro-8H-[1,2,4]triazolo[4,3-a]pyrazine, and
   substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   wherein R₁ is optionally further mono-, di-, or tri-substituted with substituents independently chosen from hydroxy, cyano, halo, C₁-C₂alkyl, and C₁-C₂alkoxy.

In some embodiments,
R₁ is chosen from
phenyl substituted with a group chosen from
   —C₁-C₆alkylene(C═O)OR₁₀,
   substituted —C₁-C₆alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylene(C═O)NR₁₀R₁₁,
   substituted —C₀-C₆alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁, chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂, and
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from
   —C₁-C₆alkylene(C═O)OR₁₀,
   substituted —C₁-C₆alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylene(C═O)NR₁₀R₁₁,
   substituted —C₀-C₆alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂ and
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁₀, R₁₁, and R₁₂ are independently chosen from hydrogen, hydroxy, C₁-C₆alkyl, C₁-C₆alkoxy, C₃-C₇cycloalkyl, and heterocycloalkyl, and
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments,
R₁ is chosen from
phenyl substituted with a group chosen from
   —C₁-C₄alkylene(C═O)OR₁₀,
   substituted —C₁-C₄alkylene(C═O)OR₁₀ chosen from mono-, di-, and tri-substituted —C₁-C₄alkylene(C═O)OR₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylene(C═O)NR₁₀R₁₁,
   substituted —C₀-C₄alkylene(C═O)NR₁₀R₁₁ chosen from mono-, di-, and tri-substituted —C₀-C₄alkylene(C═O)NR₁₀R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₁-C₄alkylNR₁₀(SO₂)R₁₁,
   substituted —C₁-C₄alkylNR₁₀(SO₂)R₁₁ chosen from mono-, di-, and tri-substituted —C₁-C₄alkylNR₁₀(SO₂)R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylNR₁₀(C═O)R₁₁,
   substituted —C₀-C₄alkylNR₁₀(C═O)R₁₁ chosen from mono-, di-, and tri-substituted —C₀-C₄alkylNR₁₀(C═O)R₁₁, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkyl(SO₂)R₁₀,
   substituted —C₀-C₄alkyl(SO₂)R₁₀ chosen from mono-, di-, and tri-substituted —C₀-C₄alkyl(SO₂)R₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylNR₁₀(C═O)NR₁₁R₁₂ and
   substituted —C₀-C₄alkylNR₁₀(C═O)NR₁₁R₁₂ chosen from mono-, di-, and tri-substituted —C₀-C₄alkylNR₁₀(C═O)NR₁₁R₁₂, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
   —C₁-C₄alkylene(C═O)OR₁₀,
   substituted —C₁-C₄alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylene(C═O)NR₁₀R₁,
   substituted —C₀-C₄alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₁-C₄alkylNR₁₀(SO₂)R₁₁,
   substituted —C₁-C₄alkylNR₁₀(SO₂)R₁₁ chosen from mono-, di-, and tri-substituted —C₁-C₄alkylNR₁₀(SO₂)R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylNR₁₀(C═O)R₁₁,
   substituted —C₀-C₄alkylNR₁₀(C═O)R₁₁ chosen from mono-, di-, and tri-substituted —C₀-C₄alkylNR₁₀(C═O)R₁₁, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkyl(SO₂)R₁₀,
   substituted —C₀-C₄alkyl(SO₂)R₁₀ chosen from mono-, di-, and tri-substituted —C₀-C₄alkyl(SO₂)R₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
   —C₀-C₄alkylNR₁₀(C═O)NR₁₁R₁₂ and
   substituted —C₀-C₄alkylNR₁₀(C═O)NR₁₁R₁₂ chosen from mono-, di-, and tri-substituted —C₀-C₄alkylNR₁₀(C═O)NR₁₁R₁₂, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkyl amino, di-C₁-C₄alkylamino, mono-C₁-C₄alkylcarboxamido, and di-C₁-C₄alkylcarboxamido,
wherein R₁₀, R₁₁, and R₁₂ are independently chosen from hydrogen, hydroxy, C₁-C₆alkyl, C₁-C₆alkoxy, C₃-C₁₀cycloalkyl, pyrrolidinyl, morpholinyl, thiomorpholinyl, piperazinyl, piperidinyl, and [1,4]diazepanyl, and
wherein R₁ is optionally further mono-, di-, or tri-substituted with substituents independently chosen from hydroxy, cyano, halo, C₁-C₂alkyl, and C₁-C₂alkoxy.

In some embodiments,
R₁ is chosen from
phenyl substituted with a group chosen from
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkyl)(C═O)—, and
         substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkyl amino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkyl)(C═O)—, and
         substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

In some embodiments,
R₁ is chosen from
phenyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
   G is chosen from
      pyrrolidinyl,
      substituted pyrrolidinyl chosen from mono-, di-, and tri-substituted pyrrolidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      morpholinyl,
      substituted morpholinyl chosen from mono-, di-, and tri-substituted morpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      thiomorpholinyl,
      substituted thiomorpholinyl chosen from mono-, di-, and tri-substituted thiomorpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkyl amino, mono-C₁-C₄alkylcarboxamido, and di-C₁-C₄alkylcarboxamido,
      piperazinyl,
      substituted piperazinyl chosen from mono-, di-, and tri-substituted piperazinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperidinyl,
      substituted piperidinyl chosen from mono-, di-, and tri-substituted piperidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      [1,4]diazepanyl,
      substituted [1,4]diazepanyl chosen from mono-, di-, and tri-substituted [1,4]diazepanyl wherein the substituents are independently chosen from halo, hydroxy, cyano, amino, oxo, —COOH, —CONH₂. C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      octahydro-pyrido[1,2-a]pyrazin-2-yl,
      substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      phenyl,
      substituted phenyl chosen from mono-, di-, and tri-substituted phenyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      imidazolyl,
      substituted imidazolyl chosen from mono-, di-, and tri-substituted imidazolyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      5,6-dihydro-8H-[1,2,4]triazolo[4,3-a]pyrazine,
      substituted 5,6-dihydro-8H-[1,2,4]triazolo[4,3-a]pyrazine chosen from mono-, di-, and tri-substituted 5,6-dihydro-8H-[1,2,4]triazolo[4,3-a]pyrazine wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
   G is chosen from
      pyrrolidinyl,
      substituted pyrrolidinyl chosen from mono-, di-, and tri-substituted pyrrolidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      morpholinyl,
      substituted morpholinyl chosen from mono-, di-, and tri-substituted morpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      thiomorpholinyl,
      substituted thiomorpholinyl chosen from mono-, di-, and tri-substituted thiomorpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkyl carboxamido, and di-C₁-C₄alkylcarboxamido,
      piperazinyl,
      substituted piperazinyl chosen from mono-, di-, and tri-substituted piperazinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, and di-C₁-C₄alkylcarboxamido,
      piperidinyl,
      substituted piperidinyl chosen from mono-, di-, and tri-substituted piperidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      [1,4]diazepanyl,
      substituted [1,4]diazepanyl chosen from mono-, di-, and tri-substituted [1,4]diazepanyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkyl carboxamido, and di-C₁-C₄alkylcarboxamido,
      octahydro-pyrido[1,2-a]pyrazin-2-yl,
      substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      phenyl,
      substituted phenyl chosen from mono-, di-, and tri-substituted phenyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      imidazolyl,
      substituted imidazolyl chosen from mono-, di-, and tri-substituted imidazolyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      5,6-dihydro-8H-[1,2,4]triazolo[4,3-a]pyrazine, and
      substituted 5,6-dihydro-8H-[1,2,4]triazolo[4,3-a]pyrazine chosen from mono-, di-, and tri-substituted 5,6-dihydro-8H-[1,2,4]triazolo[4,3-a]pyrazine wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

In some embodiments,
R₁ is chosen from
phenyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
   G is chosen from
      pyrrolidinyl,
      substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      morpholinyl,
      substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      thiomorpholinyl,
      substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperazinyl,
      substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperidinyl,
      substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      [1,4]diazepanyl,
      substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      octahydro-pyrido[1,2-a]pyrazin-2-yl,
      substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      phenyl,
      substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      imidazolyl,
      substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      5,6-dihydro-8H-[1,2,4]triazolo[4,3-a]pyrazine, and
      substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with -L-G, where
   L is chosen from
      C₁-C₂alkylene,
      substituted C₁-C₂alkylene chosen from mono-, di-, and tri-substituted C₁-C₂alkylene wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      C₀-C₂alkylene-O—,
      substituted C₀-C₂alkylene-O— chosen from mono-, di-, and tri-substituted C₀-C₂alkylene-O— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      —(C═O)—,
      —(C₁-C₂alkyl)(C═O)—, and
      substituted —(C₁-C₂alkyl)(C═O)— chosen from mono-, di-, and tri-substituted —(C₁-C₂alkyl)(C═O)— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
   G is chosen from
      pyrrolidinyl,
      substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino; mono-C₁-C₄alkylcarboxamido, and di-C₁-C₄alkylcarboxamido,
      morpholinyl,
      substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      thiomorpholinyl,
      substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperazinyl,
      substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      piperidinyl,
      substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      [1,4]diazepanyl,
      substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      octahydro-pyrido[1,2-a]pyrazin-2-yl,
      substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkyl carboxamido,
      phenyl,
      substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      imidazolyl,
      substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
      5,6-dihydro-8H-[1,2,4]triazolo[4,3-a]pyrazine, and
      substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy.

In some embodiments,
L is chosen from
   phenyl substituted with a group chosen from —CH₂—, —(C═O)—, and —(CH₂)(C═O)— and
   pyridyl substituted with a group chosen from —CH₂—, —(C═O)—, and —(CH₂)(C═O)— and
G is chosen from
   pyrrolidinyl,
   substituted pyrrolidinyl chosen from mono-, di- and tri-substituted pyrrolidinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   morpholinyl,
   substituted morpholinyl chosen from mono-, di- and tri-substituted morpholinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   thiomorpholinyl,
   substituted thiomorpholinyl chosen from mono-, di- and tri-substituted thiomorpholinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   piperazinyl,
   substituted piperazinyl chosen from mono-, di- and tri-substituted piperazinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   piperidinyl,
   substituted piperidinyl chosen from mono-, di- and tri-substituted piperidinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   [1,4]diazepanyl,
   substituted [1,4]diazepanyl chosen from mono-, di- and tri-substituted [1,4]diazepanyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
   octahydro-pyrido[1,2-a]pyrazin-2-yl, and
   substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido.

Also provided is at least one chemical entity chosen from compounds of Formula 7 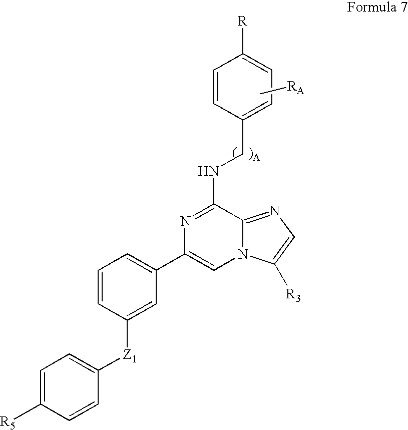
and pharmaceutically acceptable salts, solvates, crystal forms, chelates, non-covalent complexes, prodrugs, and mixtures thereof, wherein B, Z₁, and R₃ are as described for compounds of Formula 1, wherein
R₅ is chosen from isopropyl and t-butyl; and
R is chosen from
   hydroxy,
   —CHO,
   —COOH,
   —CONH₂,
   —CONHOH,
   C₂-C₆ alkenyl,
   substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₂-C₆ alkynyl,
   substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkyl,
   substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   C₁-C₆alkoxy,
   substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heterocycloalkyl,
   substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   aryl,
   substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   heteroaryl,
   substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₁-C₆alkylNR₁₀(SO₂)R₁₁,
   substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)R₁₁,
   substituted —C₀-C₆alkylNR₁₀(C═O)R₁₁, chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkyl(SO₂)R₁₀,
   substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   —C₀-C₆alkylNR₁₀(C═O)NR₁₁R₁₂,
   substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
   where R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl, and
   -L-G, where
      L is chosen from
         C₁-C₂alkylene,
         substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₀-C₂alkylene-O—,
         substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         —(C═O)—,
         —(C₁-C₂alkylene)(C═O)—, and
         substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
      and G is chosen from
         heterocycloalkyl,
         substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         C₃-C₇cycloalkyl,
         substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         aryl,
         substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
         heteroaryl, and
         substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R_{A} is optional and when present, can be present up to four times, and is independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.
   In some embodiments, R₃ is chosen from hydrogen and methyl.

Also provided is at least one chemical entity chosen from compounds of Formula 8: 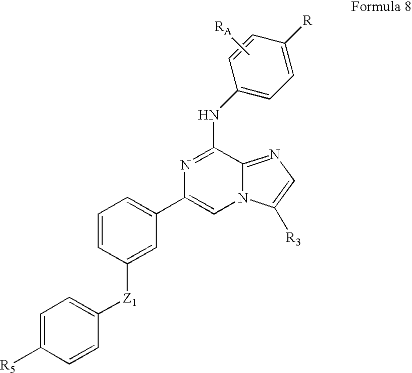
and pharmaceutically acceptable salts, solvates, crystal forms, chelates, non-covalent complexes, prodrugs, and mixtures thereof wherein R and R_{A} are as described for Formula 2, Z₁ and R₃ are as described for Formula 1, and R₅ is chosen from isopropyl and t-butyl.

Also provided is at least one chemical entity chosen from compounds of Formula 9: 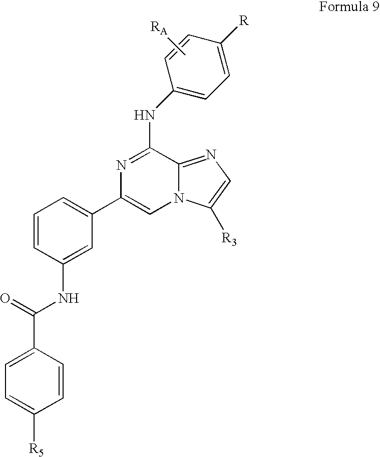
and pharmaceutically acceptable salts, solvates, crystal forms, chelates, non-covalent complexes, prodrugs, and mixtures thereof, wherein R and R_{A} are as described for Formula 2, R₃ is as described for Formula 1, and R₅ is chosen from isopropyl and t-butyl.

In some embodiments, at least one one chemical entity is chosen from
4-tert-Butyl-N-[3-(8-phenyl amino-imidazo[1,2-a]pyrazin-6-yl)-phenyl]-benzamide;
4-Isopropyl-N-[3-(8-phenylamino-imidazo[1,2-a]pyrazin-6-yl)-phenyl]-benzamide;
4-tert-Butyl-N-{3-[8-(pyridin-3-ylamino)-imidazo[1,2-a]pyrazin-6-yl]-phenyl}-benzamide;
4-tert-Butyl-N-{3-[8-(4-methoxy-phenylamino)-imidazo[1,2-a]pyrazin-6-yl]-phenyl}-benzamide;
4-tert-Butyl-N-{3-[8-(pyridin-4-ylamino)-imidazo[1,2-a]pyrazin-6-yl]-phenyl}-benzamide;
4-tert-Butyl-N-{3-[8-(4-fluoro-phenylamino)-imidazo[1,2-a]pyrazin-6-yl]-phenyl}-benzamide;
4-tert-Butyl-N-{3-[8-(3-fluoro-phenylamino)-imidazo[1,2-a]pyrazin-6-yl]-phenyl}-benzamide;
4-{6-[3-(4-tert-Butyl-benzoylamino)-phenyl]-imidazo[1,2-a]pyrazin-8-ylamino}-benzoic acid;
4-tert-Butyl-N-{3-[8-(3-methoxy-phenylamino)-imidazo[1,2-a]pyrazin-6-yl]-phenyl}-benzamide;
4-tert-Butyl-N-{3-[8-(6-methoxy-pyridin-3-ylamino)-imidazo[1,2-a]pyrazin-6-yl]-phenyl}-benzamide;
3-{6-[3-(4-tert-Butyl-benzoylamino)-phenyl]-imidazo[1,2-a]pyrazin-8-ylamino}-benzoic acid;
4-{6-[3-(4-Isopropyl-benzoylamino)-phenyl]-imidazo[1,2-a]pyrazin-8-ylamino}-benzoic acid;
4-tert-Butyl-N-{3-[8-(4-cyano-phenylamino)-imidazo[1,2-a]pyrazin-6-yl]-phenyl}-benzamide;
4-{6-[3-(4-tert-Butyl-benzoylamino)-phenyl]-imidazo[1,2-a]pyrazin-8-ylamino}-benzamide;
4-tert-Butyl-N-{3-[8-(4-morpholin-4-yl-phenylamino)-imidazo[1,2-a]pyrazin-6-yl]-phenyl}-benzamide;
3-{6-[3-(4-tert-Butyl-benzoylamino)-phenyl]-imidazo[1,2-a]pyrazin-8-ylamino}-benzoic acid;
4-tert-Butyl-N-{3-[8-(3-fluoro-4-methoxy-phenylamino)-imidazo[1,2-a]pyrazin-6-yl]-phenyl}-benzamide;
4-{6-[3-(4-tert-Butyl-benzoylamino)-phenyl]-imidazo[1,2-a]pyrazin-8-ylamino}-benzoic N-Methyl amide;
4-tert-Butyl-N-{3-[8-(4-piperazin-1-yl-phenylamino)-imidazo[1,2-a]pyrazin-6-yl]-phenyl}-benzamide;
4-tert-Butyl-N-[3-(8-m-tolylamino-imidazo[1,2-a]pyrazin-6-yl)-phenyl]-benzamide;
(4-{6-[3-(4-tert-Butyl-benzoylamino)-phenyl]-imidazo[1,2-a]pyrazin-8-ylamino}-phenyl)-acetic acid methyl ester;
(4-{6-[3-(4-tert-Butyl-benzoylamino)-phenyl]-imidazo[1,2-a]pyrazin-8-ylamino}-phenyl)-acetic acid;
4-tert-Butyl-N-(3-{8-[4-(piperazine-1-carbonyl)-phenylamino]-imidazo[1,2-a]pyrazin-6-yl}-phenyl)-benzamide;
4-tert-Butyl-N-(3-{8-[4-(2-methoxy-ethoxymethoxy)-phenylamino]-imidazo[1,2-a]pyrazin-6-yl}-phenyl)-benzamide;
4-tert-Butyl-N-{3-[8-(4-hydroxy-phenylamino)-imidazo[1,2-a]pyrazin-6-yl]-phenyl}-benzamide;
4-tert-Butyl-N-{3-[8-(6-piperazin-1-yl-pyridin-3-ylamino)-imidazo[1,2-a]pyrazin-6-yl]-phenyl}-benzamide;
4-tert-Butyl-N-(3-{8-[4-(4-methyl-piperazin-1-yl)-phenylamino]-imidazo[1,2-a]pyrazin-6-yl}-phenyl)-benzamide;
4-tert-Butyl-N-{3-[8-(4-[1,4]diazepan-1-yl-phenylamino)-imidazo[1,2-a]pyrazin-6-yl]-phenyl}-benzamide;
4-tert-Butyl-N-(3-{8-[4-(4-oxo-piperidin-1-yl)-phenylamino]-imidazo[1,2-a]pyrazin-6-yl}-phenyl)-benzamide;
(4-{6-[3-(4-tert-Butyl-benzoylamino)-phenyl]-imidazo[1,2-a]pyrazin-8-ylamino}-benzoylamino)-acetic acid methyl ester;
(4-{6-[3-(4-tert-Butyl-benzoylamino)-phenyl]-imidazo[1,2-a]pyrazin-8-ylamino}-benzoylamino)-acetic acid;
N-(3-{8-[4-(4-Acetyl-piperazin-1-yl)-phenylamino]-imidazo[1,2-a]pyrazin-6-yl}-phenyl)-4-tert-butyl-benzamide;
4-tert-Butyl-N-(3-{8-[4-(3-methyl-piperazin-1-yl)-phenylamino]-imidazo[1,2-a]pyrazin-6-yl}-phenyl)-benzamide;
4-{6-[3-(4-tert-Butyl-benzoylamino)-phenyl]-imidazo[1,2-a]pyrazin-8-ylamino}-benzoic hydroxyl amide;
4-tert-Butyl-N-{3-[8-(4-hydroxymethyl-phenylamino)-imidazo[1,2-a]pyrazin-6-yl]-phenyl}-benzamide;
4-tert-Butyl-N-{3-[8-(4-piperazin-1-ylmethyl-phenylamino)-imidazo[1,2-a]pyrazin-6-yl]-phenyl}-benzamide;
4-{6-[3-(4-Bromo-3-methyl-benzoylamino)-phenyl]-imidazo[1,2-a]pyrazin-8-ylamino}-benzoic acid;
4-tert-Butyl-N-{3-[8-(3-methyl-4-piperazin-1-yl-phenylamino)-imidazo[1,2-a]pyrazin-6-yl]-phenyl}-benzamide;
4-tert-Butyl-N-(3-{8-[4-(3-methyl-piperazin-1-yl)-phenylamino]-imidazo[1,2-a]pyrazin-6-yl}-phenyl)-benzamide;
4-tert-Butyl-N-{3-[8-(3-hydroxymethyl-phenylamino)-imidazo[1,2-a]pyrazin-6-yl]-phenyl}-benzamide;
4-tert-Butyl-N-(3-{8-[3-(3-methyl-piperazin-1-ylmethyl)-phenylamino]-imidazo[1,2-a]pyrazin-6-yl}-phenyl)-benzamide;
4-tert-Butyl-N-(3-{8-[3-(3-methyl-piperazin-1-ylmethyl)-phenylamino]-imidazo[1,2-a]pyrazin-6-yl}-phenyl)-benzamide;
4-tert-Butyl-N-(3-{8-[3-(3,5-dimethyl-piperazin-1-ylmethyl)-phenylamino]-imidazo[1,2-a]pyrazin-6-yl}-phenyl)-benzamide;
4-tert-Butyl-N-(3-{8-[4-(3,5-dimethyl-piperazin-1-yl)-phenylamino]-imidazo[1,2-a]pyrazin-6-yl}-phenyl)-benzamide;
4-({6-[3-(4-tert-Butyl-benzoylamino)-phenyl]-imidazo[1,2-a]pyrazin-8-ylamino}-methyl)-benzoic acid;
4-tert-Butyl-N-(3-{8-[4-(3-methyl-piperazin-1-yl)-phenylamino]-imidazo[1,2-a]pyrazin-6-yl}-phenyl)-benzamide;
4-(1-Hydroxy-1-methyl-ethyl)-N-[3-(8-phenylamino-imidazo[1,2-a]pyrazin-6-yl)-phenyl]-benzamide;
(3-{6-[3-(4-tert-Butyl-benzoylamino)-phenyl]-imidazo[1,2-a]pyrazin-8-ylamino}-phenyl)-acetic acid;
N-[3-(8-Phenylamino-imidazo[1,2-a]pyrazin-6-yl)-phenyl]-terephthalamic acid methyl ester;
N-[3-(8-Phenylamino-imidazo[1,2-a]pyrazin-6-yl)-phenyl]-terephthalamic acid;
2-(4-{6-[3-(4-tert-Butyl-benzoylamino)-phenyl]-imidazo[1,2-a]pyrazin-8-ylamino}-phenyl)-propionic acid ethyl ester;
2-(4-{6-[3-(4-tert-Butyl-benzoylamino)-phenyl]-imidazo[1,2-a]pyrazin-8-ylamino}-phenyl)-propionic acid;
4-{6-[3-(4-tert-Butyl-benzoylamino)-phenyl]-imidazo[1,2-a]pyrazin-8-ylamino}-2-methoxy-benzoic acid methyl ester;
4-{6-[3-(4-tert-Butyl-benzoylamino)-phenyl]-imidazo[1,2-a]pyrazin-8-ylamino}-2-methoxy-benzoic acid;
4-tert-Butyl-N-{3-[8-(3-methylcarbamoylmethyl-phenylamino)-imidazo[1,2-a]pyrazin-6-yl]-phenyl}-benzamide;
4-tert-Butyl-N-{3-[8-(4-methylcarbamoylmethyl-phenylamino)-imidazo[1,2-a]pyrazin-6-yl]-phenyl}-benzamide;
4-tert-Butyl-N-{3-[8-(4-cyclooctylcarbamoylmethyl-phenylamino)-imidazo[1,2-a]pyrazin-6-yl]-phenyl}-benzamide;
4-tert-Butyl-N-[3-(8-{4-[(diisopropylcarbamoyl)-methyl]-phenylamino}-imidazo[1,2-a]pyrazin-6-yl)-phenyl]-benzamide;
4-tert-Butyl-N-{3-[8-(4-carbamoylmethyl-phenylamino)-imidazo[1,2-a]pyrazin-6-yl]-phenyl}-benzamide;
3-(4-{6-[3-(4-tert-Butyl-benzoylamino)-phenyl]-imidazo[1,2-a]pyrazin-8-ylamino}-phenyl)-propionic acid;
4-tert-Butyl-N-(3-{8-[4-(2-hydroxy-ethyl)-phenylamino]-imidazo[1,2-a]pyrazin-6-yl}-phenyl)-benzamide;
4-tert-Butyl-N-(3-{8-[4-(2-methylamino-ethyl)-phenylamino]-imidazo[1,2-a]pyrazin-6-yl}-phenyl)-benzamide;
4-tert-Butyl-N-{3-[8-(4-methylaminomethyl-phenylamino)-imidazo[1,2-a]pyrazin-6-yl]-phenyl}-benzamide;
4-{6-[3-(4-tert-Butyl-benzoylamino)-phenyl]-imidazo[1,2-a]pyrazin-8-ylamino}-2-hydroxy-benzoic acid;
4-tert-Butyl-N-(3-{8-[4-(1-hydroxy-1-methyl-ethyl)-phenylamino]-imidazo[1,2-a]pyrazin-6-yl}-phenyl)-benzamide;
4-tert-Butyl-N-(3-{8-[4-(2-hydroxy-ethoxy)-phenylamino]-imidazo[1,2-a]pyrazin-6-yl}-phenyl)-benzamide;
4-tert-Butyl-N-(3-{8-[4-(4-methyl-piperazine-1-carbonyl)-phenylamino]-imidazo[1,2-a]pyrazin-6-yl}-phenyl)-benzamide;
4-tert-Butyl-N-(3-{8-[4-(2-methylamino-ethoxy)-phenylamino]-imidazo[1,2-a]pyrazin-6-yl}-phenyl)-benzamide;
4-tert-Butyl-N-(3-{8-[4-(2-dimethylamino-ethoxy)-phenylamino]-imidazo[1,2-a]pyrazin-6-yl}-phenyl)-benzamide;
4-{6-[3-(4-tert-Butyl-benzoylamino)-phenyl]-imidazo[1,2-a]pyrazin-8-ylamino}-benzoic methoxy amide;
4-{6-[3-(4-tert-Butyl-benzoylamino)-phenyl]-imidazo[1,2-a]pyrazin-8-ylamino}-benzoic hyrdoxyl methyl amide;
4-tert-Butyl-N-(3-{8-[4-([1,4]diazepane-1-carbonyl)-phenylamino]-imidazo[1,2-a]pyrazin-6-yl}-phenyl)-benzamide;
4-tert-Butyl-N-(3-{8-[4-(pyrrolidine-1-carbonyl)-phenylamino]-imidazo[1,2-a]pyrazin-6-yl}-phenyl)-benzamide;
4-tert-Butyl-N-(3-{8-[4-(morpholine-4-carbonyl)-phenylamino]-imidazo[1,2-a]pyrazin-6-yl}-phenyl)-benzamide;
4-tert-Butyl-N-(3-{8-[4-(ethoxyamine-4-carbonyl)-phenylamino]-imidazo[1,2-a]pyrazin-6-yl}-phenyl)-benzamide;
4-tert-Butyl-N-(3-{8-[4-(dimethylamine-4-carbonyl)-phenylamino]-imidazo[1,2-a]pyrazin-6-yl}-phenyl)-benzamide;
4-tert-Butyl-N-(3-{8-[4-(methylethyl amine-4-carbonyl)-phenylamino]-imidazo[1,2-a]pyrazin-6-yl}-phenyl)-benzamide;
4-tert-Butyl-N-(3-{8-[4-(3-oxo-piperazine-1-carbonyl)-phenylamino]-imidazo[1,2-a]pyrazin-6-yl}-phenyl)-benzamide;
4-tert-Butyl-N-(3-{8-[N,N,N′-Trimethyl-ethane-1,2-diamine-4-carbonyl)-phenylamino]-imidazo[1,2-a]pyrazin-6-yl}-phenyl)-benzamide;
4-tert-Butyl-N-(3-{8-[4-(4-methyl-[1,4]diazepane-1-carbonyl)-phenylamino]-imidazo[1,2-a]pyrazin-6-yl}-phenyl)-benzamide;
4-tert-Butyl-N-(3-{8-[4-(N-methyl ethoxyamine-4-carbonyl)-phenylamino]-imidazo[1,2-a]pyrazin-6-yl}-phenyl)-benzamide;
4-tert-Butyl-N-(3-{8-[4-(4-ethyl-piperazine-1-carbonyl)-phenylamino]-imidazo[1,2-a]pyrazin-6-yl}-phenyl)-benzamide;
4-{6-[3-(4-tert-Butyl-benzoylamino)-phenyl]-imidazo[1,2-a]pyrazin-8-ylamino}-benzoic benzyl amide;
4-tert-Butyl-N-(3-{8-[4-(4-methyl-piperazin-1-ylmethyl)-phenylamino]-imidazo[1,2-a]pyrazin-6-yl}-phenyl)-benzamide;
4-tert-Butyl-N-(3-{8-[4-(4-ethyl-piperazin-1-ylmethyl)-phenylamino]-imidazo[1,2-a]pyrazin-6-yl}-phenyl)-benzamide;
4-tert-Butyl-N-{3-[8-(4-[1,4]diazepan-1-ylmethyl-phenyl amino)-imidazo[1,2-a]pyrazin-6-yl]-phenyl}-benzamide;
4-tert-Butyl-N-(3-{8-[4-(3-methyl-piperazin-1-ylmethyl)-phenylamino]-imidazo[1,2-a]pyrazin-6-yl}-phenyl)-benzamide;
4-tert-Butyl-N-{3-[8-(4-dimethylaminomethyl-phenylamino)-imidazo[1,2-a]pyrazin-6-yl]-phenyl}-benzamide;
4-tert-Butyl-N-[3-(8-{4-[(ethyl-methyl-amino)-methyl]-phenylamino}-imidazo[1,2-a]pyrazin-6-yl)-phenyl]-benzamide;
4-tert-Butyl-N-{3-[8-(4-{[(2-methoxy-ethyl)-methyl-amino]-methyl}-phenylamino)-imidazo[1,2-a]pyrazin-6-yl]-phenyl}-benzamide;
4-tert-Butyl-N-[3-(8-{4-[(2-methoxy-ethylamino)-methyl]-phenylamino}-imidazo[1,2-a]pyrazin-6-yl)-phenyl]-benzamide;
4-tert-Butyl-N-{3-[8-(4-{[(2-dimethylamino-ethyl)-methyl-amino]-methyl}-phenylamino)-imidazo[1,2-a]pyrazin-6-yl]-phenyl}-benzamide;
N-(3-{8-[4-(4-Acetyl-piperazine-1-carbonyl)-phenylamino]-imidazo[1,2-a]pyrazin-6-yl}-phenyl)-4-tert-butyl-benzamide;
4-tert-Butyl-N-{3-[8-(4-{[(2-hydroxy-ethyl)-methyl-amino]-methyl}-phenylamino)-imidazo[1,2-a]pyrazin-6-yl]-phenyl}-benzamide;
4-tert-Butyl-N-[3-(8-{4-[(2-hydroxy-ethylamino)-methyl]-phenylamino}-imidazo[1,2-a]pyrazin-6-yl)-phenyl]-benzamide;
4-tert-Butyl-N-(3-{8-[4-(4-methyl-[1,4]diazepan-1-ylmethyl)-phenylamino]-imidazo[1,2-a]pyrazin-6-yl}-phenyl)-benzamide;
N-[3-(8-{4-[2-(Acetyl-methyl-amino)-ethoxy]-phenylamino}-imidazo[1,2-a]pyrazin-6-yl)-phenyl]-4-tert-butyl-benzamide;
4-(4-{6-[3-(4-tert-Butyl-benzoylamino)-phenyl]-imidazo[1,2-a]pyrazin-8-ylamino}-phenyl)-piperazine-1-carboxylic acid ethyl ester;
N-(3-{8-[4-(4-Acetyl-piperazin-1-ylmethyl)-phenylamino]-imidazo[1,2-a]pyrazin-6-yl}-phenyl)-4-tert-butyl-benzamide;
4-tert-Butyl-N-{3-[8-(4-imidazol-1-ylmethyl-phenylamino)-imidazo[1,2-a]pyrazin-6-yl]-phenyl}-benzamide;
4-tert-Butyl-N-{3-[8-(4-pyrrolidin-1-ylmethyl-phenylamino)-imidazo[1,2-a]pyrazin-6-yl]-phenyl}-benzamide;
N-[3-(8-{4-[(Acetyl-methyl-amino)-methyl]-phenylamino}-imidazo[1,2-a]pyrazin-6-yl)-phenyl]-4-tert-butyl-benzamide;
4-tert-Butyl-N-[3-(8-{4-[(methanesulfonyl-methyl-amino)-methyl]-phenylamino}-imidazo[1,2-a]pyrazin-6-yl)-phenyl]-benzamide;
4-tert-Butyl-N-(3-{8-[4-(3-isopropyl-1-methyl-ureidomethyl)-phenylamino]-imidazo[1,2-a]pyrazin-6-yl}-phenyl)-benzamide;
4-Isopropyl-N-(3-{8-[4-(morpholine-4-carbonyl)-phenylamino]-imidazo[1,2-a]pyrazin-6-yl}-phenyl)-benzamide;
N-{3-[8-(4-{[(2-Hydroxy-ethyl)-methyl-amino]-carbonyl}-phenylamino)-imidazo[1,2-a]pyrazin-6-yl]-phenyl}-4-isopropyl-benzamide;
N-(3-{8-[4-(Morpholine-4-carbonyl)-phenylamino]-imidazo[1,2-a]pyrazin-6-yl}-phenyl)-benzamide;
4-Methyl-N-(3-{8-[4-(morpholine-4-carbonyl)-phenylamino]-imidazo[1,2-a]pyrazin-6-yl}-phenyl)-benzamide;
4-Ethyl-N-(3-{8-[4-(morpholine-4-carbonyl)-phenylamino]-imidazo[1,2-a]pyrazin-6-yl}-phenyl)-benzamide;
4-Fluoro-N-(3-{8-[4-(morpholine-4-carbonyl)-phenylamino]-imidazo[1,2-a]pyrazin-6-yl}-phenyl)-benzamide;
N-(3-{8-[4-(Morpholine-4-carbonyl)-phenylamino]-imidazo[1,2-a]pyrazin-6-yl}-phenyl)-4-trifluoromethyl-benzamide;
4-tert-Butyl-N-(3-{8-[4-(5,6-dihydro-8H-imidazo[1,2-a]pyrazine-7-carbonyl)-phenylamino]-imidazo[1,2-a]pyrazin-6-yl}-phenyl)-benzamide;
4-tert-Butyl-N-(3-{8-[4-(3-oxo-piperazin-1-ylmethyl)-phenylamino]-imidazo[1,2-a]pyrazin-6-yl}-phenyl)-benzamide;
1-(4-{6-[3-(4-tert-Butyl-benzoylamino)-phenyl]-imidazo[1,2-a]pyrazin-8-yl amino}-benzyl)-pyrrolidine-2-carboxylic acid methyl ester;
N-(3-{8-[4-(Morpholine-4-carbonyl)-phenylamino]-imidazo[1,2-a]pyrazin-6-yl}-phenyl)-terephthalamic acid methyl ester;
N-(3-{8-[4-(Morpholine-4-carbonyl)-phenylamino]-imidazo[1,2-a]pyrazin-6-yl}-phenyl)-terephthalamic acid;
N-(3-{8-[4-(4-Acetyl-piperazin-1-yl)-phenylamino]-imidazo[1,2-a]pyrazin-6-yl}-phenyl)-4-ethyl-benzamide;
N-(3-{8-[4-(4-Acetyl-piperazin-1-yl)-phenylamino]-imidazo[1,2-a]pyrazin-6-yl}-phenyl)-4-trifluoromethyl-benzamide;
4-tert-Butyl-N-(3-{8-[4-(3-methyl-5,6-dihydro-8H-[1,2,4]triazolo[4,3-a]pyrazin-7-ylmethyl)-phenylamino]-imidazo[1,2-a]pyrazin-6-yl}-phenyl)-benzamide;
4-tert-Butyl-N-(3-{8-[4-(3-methyl-5,6-dihydro-8H-[1,2,4]triazolo[4,3-a]pyrazine-7-carbonyl)-phenylamino]-imidazo[1,2-a]pyrazin-6-yl}-phenyl)-benzamide;
N-(3-{8-[4-(4-Acetyl-piperazin-1-yl)-phenylamino]-imidazo[1,2-a]pyrazin-6-yl}-phenyl)-4-isopropyl-benzamide;
N-(3-{8-[4-(4-Acetyl-piperazine-1-carbonyl)-phenylamino]-imidazo[1,2-a]pyrazin-6-yl}-phenyl)-4-isopropyl-benzamide;
1-(4-{6-[3-(4-tert-Butyl-benzoylamino)-phenyl]-imidazo[1,2-a]pyrazin-8-ylamino}-benzyl)-pyrrolidine-2-carboxylic acid;
4-Isopropyl-N-(3-{8-[4-(3-methyl-5,6-dihydro-8H-[1,2,4]triazolo[4,3-a]pyrazine-7-carbonyl)-phenylamino]-imidazo[1,2-a]pyrazin-6-yl}-phenyl)-benzamide;
N-(3-{8-[4-(5,6-Dihydro-8H-imidazo[1,2-a]pyrazine-7-carbonyl)-phenylamino]-imidazo[1,2-a]pyrazin-6-yl}-phenyl)-4-isopropyl-benzamide;
4-Isopropyl-N-(3-{8-[4-(3-oxo-piperazine-1-carbonyl)-phenylamino]-imidazo[1,2-a]pyrazin-6-yl}-phenyl)-benzamide;
4-Isopropyl-N-(3-{8-[4-(4-methyl-piperazine-1-carbonyl)-phenylamino]-imidazo[1,2-a]pyrazin-6-yl}-phenyl)-benzamide;
4-Isopropyl-N-(3-{8-[4-(3-oxo-piperazin-1-ylmethyl)-phenylamino]-imidazo[1,2-a]pyrazin-6-yl}-phenyl)-benzamide;
N-(3-{8-[4-(4-Acetyl-piperazin-1-ylmethyl)-phenyl amino]-imidazo[1,2-a]pyrazin-6-yl}-phenyl)-4-isopropyl-benzamide;
N-(3-{8-[4-(Morpholine-4-carbonyl)-phenylamino]-imidazo[1,2-a]pyrazin-6-yl}-phenyl)-isonicotinamide;
N-(3-{8-[4-(Morpholine-4-carbonyl)-phenylamino]-imidazo[1,2-a]pyrazin-6-yl}-phenyl)-nicotinamide;
4-tert-Butyl-N-(3-{8-[4-(2-morpholin-4-yl-2-oxo-ethyl)-phenylamino]-imidazo[1,2-a]pyrazin-6-yl}-phenyl)-benzamide;
4-tert-Butyl-N-(3-{8-[3-(2-morpholin-4-yl-2-oxo-ethyl)-phenylamino]-imidazo[1,2-a]pyrazin-6-yl}-phenyl)-benzamide;
4-Isopropyl-N-{3-[8-(4-morpholin-4-ylmethyl-phenyl amino)-imidazo[1,2-a]pyrazin-6-yl]-phenyl}-benzamide;
4-tert-Butyl-N-{3-[8-(4-morpholin-4-ylmethyl-phenylamino)-imidazo[1,2-a]pyrazin-6-yl]-phenyl}-benzamide;
N-(3-{8-[4-(Morpholine-4-carbonyl)-phenylamino]-imidazo[1,2-a]pyrazin-6-yl}-phenyl)-2-phenyl-acetamide;
N-(4-tert-Butyl-phenyl)-3-{8-[4-(morpholine-4-carbonyl)-phenylamino]-imidazo[1,2-a]pyrazin-6-yl}-benzamide;
N-(3-Chloro-benzyl)-3-{8-[4-(morpholine-4-carbonyl)-phenylamino]-imidazo[1,2-a]pyrazin-6-yl}-benzamide;
4-Cyano-N-(3-{8-[4-(morpholine-4-carbonyl)-phenylamino]-imidazo[1,2-a]pyrazin-6-yl}-phenyl)-benzamide;
6-Dimethylamino-N-(3-{8-[4-(morpholine-4-carbonyl)-phenylamino]-imidazo[1,2-a]pyrazin-6-yl}-phenyl)-nicotinamide;
N-Methyl-N′-(3-{8-[4-(morpholine-4-carbonyl)-phenyl amino]-imidazo[1,2-a]pyrazin-6-yl}-phenyl)-terephthalamide;
N,N-Dimethyl-N′-(3-{8-[4-(morpholine-4-carbonyl)-phenylamino]-imidazo[1,2-a]pyrazin-6-yl}-phenyl)-terephthalamide;
4-Acetyl-N-(3-{8-[4-(morpholine-4-carbonyl)-phenylamino]-imidazo[1,2-a]pyrazin-6-yl}-phenyl)-benzamide;
4-(1H-Imidazol-2-yl)-N-(3-{8-[4-(morpholine-4-carbonyl)-phenylamino]-imidazo[1,2-a]pyrazin-6-yl}-phenyl)-benzamide;
2-(3-Isopropyl-phenoxy)-N-(3-{8-[4-(morpholine-4-carbonyl)-phenylamino]-imidazo[1,2-a]pyrazin-6-yl}-phenyl)-acetamide;
6-tert-Butyl-N-(3-{8-[4-(morpholine-4-carbonyl)-phenylamino]-imidazo[1,2-a]pyrazin-6-yl}-phenyl)-nicotinamide;
4-(6-{3-[(6-tert-Butyl-pyridine-3-carbonyl)-amino]-phenyl}-imidazo[1,2a]pyrazin-8-ylamino)-benzoic acid;
4-{6-[5-(4-tert-Butyl-benzoylamino)-pyridin-3-yl]-imidazo[1,2-a]pyrazin-8-ylamino}-benzoic acid;
4-tert-Butyl-N-(3-{8-[4-(octahydro-pyrido[1,2-a]pyrazine-2-carbonyl)-phenylamino]-imidazo[1,2-a]pyrazin-6-yl}-phenyl)-benzamide;
4-tert-Butyl-N-[3-(8-{4-[1-methyl-2-(octahydro-pyrido[1,2-a]pyrazin-2-yl)-2-oxo-ethyl]-phenylamino}-imidazo[1,2-a]pyrazin-6-yl)-phenyl]-benzamide;
Thiazole-4-carboxylic acid (3-{8-[4-(morpholine-4-carbonyl)-phenylamino]-imidazo[1,2a]pyrazin-6-yl}-phenyl)-amide;
[1,2,3]Thiadiazole-4-carboxylic acid (3-{8-[4-(morpholine-4-carbonyl)-phenylamino]-imidazo[1,2-a]pyrazin-6-yl}-phenyl)-amide;
Isoxazole-5-carboxylic acid (3-{8-[4-(morpholine-4-carbonyl)-phenylamino]-imidazo[1,2-a]pyrazin-6-yl}-phenyl)-amide;
6-Chloro-N-(3-{8-[4-(morpholine-4-carbonyl)-phenyl amino]-imidazo[1,2-a]pyrazin-6-yl}-phenyl)-nicotinamide;
4-Hydroxy-6-methyl-N-(3-{8-[4-(morpholine-4-carbonyl)-phenylamino]-imidazo[1,2-a]pyrazin-6-yl}-phenyl)-nicotinamide;
   and pharmaceutically acceptable salts, solvates, crystal forms, chelates, non-covalent complexes, prodrugs, and mixtures thereof.

In some embodiments, the chemical entities of the invention can be administered as the neat chemical. In some embodiments, the chemical entities can be administered as a pharmaceutical composition or formulation. Accordingly, some embodiments of the invention provide pharmaceutical formulations comprising at least one chemical entity of the invention together with at least one pharmaceutically acceptable vehicle chosen from carriers, excipients, and adjuvants.

Pharmaceutical carriers should be of sufficiently high purity and sufficiently low toxicity to render them suitable for administration to the animal being treated. The carrier can be inert or it can possess pharmaceutical benefits. The amount of carrier employed in conjunction with the compound is sufficient to provide a practical quantity of material for administration per unit dose of the compound.

Exemplary pharmaceutically acceptable carriers include sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; synthetic oils; vegetable oils, such as peanut oil, cottonseed oil, sesame oil, olive oil, and corn oil; polyols such as propylene glycol, glycerine, sorbitol, mannitol, and polyethylene glycol; alginic acid; phosphate buffer solutions; emulsifiers, such as the TWEENS; wetting agents, such sodium lauryl sulfate; coloring agents; flavoring agents; tableting agents; stabilizers; antioxidants; preservatives; pyrogen-free water; isotonic saline; and phosphate buffer solutions.

Optional active agents may be included in a pharmaceutical composition, which do not substantially interfere with the activity of the compound of the present invention.

Effective concentrations of at least one chemical entity of the invention can be mixed with a suitable pharmaceutically acceptable vehicle. In instances in which the chemical entities exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate.

Upon mixing or addition of at least one chemical entity, the resulting mixture may be a solution, suspension, emulsion or the like. The form of the resulting mixture depends upon a number of factors, including the intended mode of administration and the solubility of the compound in the chosen vehicle. The effective concentration sufficient for ameliorating the symptoms of the disease treated may be empirically determined.

Chemical entities provided herein may, for instance, be administered orally, topically, parenterally, intravenously, by intramuscular injection, by inhalation or spray, sublingually, transdermally, via buccal administration, rectally, as an ophthalmic solution, or by other means, in dosage unit formulations.

Dosage formulations suitable for oral use can be chosen from tablets, troches, lozenges, aqueous suspensions, oily suspensions, dispersible powders, granules, emulsions, hard capsules, soft capsules, syrups, and elixirs. Compositions intended for oral use may be prepared according to any method known to the art for the manufacture of pharmaceutical compositions and such compositions may contain one or more agents, such as sweetening agents, flavoring agents, coloring agents and preserving agents, in order to provide pharmaceutically acceptable and palatable preparations. Oral formulations contain from 0.1 to 99% of at least one chemical entity provided herein and, in some embodiments, at least 5% (weight %) of at least one chemical entity provided herein. Some embodiments contain from 25% to 50% or from 5% to 75% of at least one chemical entity provided herein.

Orally administered compositions can be chosen from liquid solutions, emulsions, suspensions, powders, granules, elixirs, tinctures, and syrups. The pharmaceutically acceptable vehicles suitable for preparation of such compositions are well known in the art. Oral formulations may contain preservatives, flavoring agents, sweetening agents, such as sucrose or saccharin, taste-masking agents, and coloring agents.

Examplary components of carriers for syrups, elixirs, emulsions and suspensions include ethanol, glycerol, propylene glycol, polyethylene glycol, liquid sucrose, sorbitol and water. Syrups and elixirs may be formulated with sweetening agents, for example glycerol, propylene glycol, sorbitol or sucrose. Such formulations may also contain a demulcent.

Chemical entities provided herein can be incorporated into oral liquid preparations chosen from aqueous suspensions, oily suspensions, solutions, emulsions, syrups, and elixirs. Moreover, formulations containing these compounds can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monsoleate, and acacia), non-aqueous vehicles, which can include edible oils (e.g., almond oil, fractionated coconut oil, silyl esters, propylene glycol and ethyl alcohol), and preservatives (e.g., methyl p-hydroxybenzoate, propyl p-hydroxybenzoate and sorbic acid).

For a suspension, examplary suspending agents include methylcellulose, sodium carboxymethyl cellulose, AVICEL RC-591, tragacanth and sodium alginate; example wetting agents include lecithin and polysorbate 80; and examplary preservatives include methyl paraben and sodium benzoate.

Aqueous suspensions contain the active material(s) in admixture with excipients suitable for the manufacture of aqueous suspensions. Such excipients can be suspending agents, for example sodium carboxymethylcellulose, methylcellulose, hydropropylmethylcellulose, sodium alginate, polyvinylpyrrolidone, gum tragacanth and gum acacia; dispersing and wetting agents; naturally-occurring phosphatides, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol substitute, or condensation products of ethylene oxide with partial esters derived from fatty acids and hexitol anhydrides, for example polyethylene sorbitan substitute. The aqueous suspensions may also contain one or more preservatives, for example ethyl p-hydroxybenzoate, and n-propyl p-hydroxybenzoate.

Oily suspensions may be formulated by suspending the active ingredients in a vegetable oil, for example peanut oil, olive oil, sesame oil and coconut oil, or in a mineral oil such as liquid paraffin. The oily suspensions may contain a thickening agent, for example beeswax, hard paraffin and cetyl alcohol. Sweetening agents such as those set forth above, and flavoring agents may be added to provide palatable oral preparations. These compositions may be preserved by the addition of an anti-oxidant such as ascorbic acid.

Pharmaceutical compositions of the invention may also be in the form of oil-in-water emulsions. The oily phase may be a vegetable oil, for example olive oil and peanut oil, or a mineral oil, for example liquid paraffin and mixtures of these. Suitable emulsifying agents may be naturally-occurring gums, for example gum acacia and gum tragacanth, naturally-occurring phosphatides, for example soy bean, lecithin, esters and partial esters derived from fatty acids and hexitol, anhydrides, for example sorbitan monoleate, and condensation products of the said partial esters with ethylene oxide, for example polyoxyethylene sorbitan monoleate.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above.

Tablets can comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, can be useful adjuvants for chewable tablets. Capsules (including time release and sustained release formulations) may comprise, for instance, one or more solid diluents disclosed above. The selection of carrier components may depend on secondary considerations like taste, cost, and shelf stability.

Such compositions may also be coated by conventional methods, for instance with pH or time-dependent coatings, such that the subject compound is released in the gastrointestinal tract in the vicinity of the desired topical application, or at various times to extend the desired action. Such dosage forms may include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, for example, calcium carbonate, calcium phosphate and kaolin, or as soft gelatin capsules wherein the active ingredient is mixed with water or an oil medium, for example peanut oil, liquid paraffin and olive oil.

Pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution, and isotonic sodium chloride solution. In addition, sterile, fixed oils can be employed as a solvent or suspending medium. For this purpose any bland fixed oil may be employed including synthetic mono- or diglycerides. In addition, fatty acids such as oleic acid are useful in the preparation of injectables.

Compounds of the invention may be administered parenterally in a sterile medium. Parenteral administration includes subcutaneous injections, intravenous, intramuscular, intrathecal injection or infusion techniques. The compound or compounds of the invention, depending on the vehicle and concentration used, can either be suspended or dissolved in the vehicle. Advantageously, adjuvants such as local anesthetics, preservatives and buffering agents can be dissolved in the vehicle. In many compositions for parenteral administration the carrier comprises at least 90% by weight of the total composition. Example carriers for parenteral administration include propylene glycol, ethyl oleate, pyrrolidone, ethanol, and sesame oil.

Compounds of the invention may also be administered in the form of suppositories for rectal administration of the drug. These compositions can be prepared by mixing the drug with a suitable non-irritating excipient that is solid at ordinary temperatures but liquid at rectal temperature and will therefore melt in the rectum to release the drug. Such materials include cocoa butter and polyethylene glycols.

Compounds of the invention may be formulated for local or topical application, such as for topical application to the skin and mucous membranes, such as in the eye, in the form of gels, creams, and lotions and for application to the eye. Topical compositions of the present invention may be in any form including, for example, solutions, creams, ointments, gels, lotions, milks, cleansers, moisturizers, sprays, and skin patches.

Such solutions may be formulated as 0.01%-10% isotonic solutions, pH from 5 to 7, with appropriate salts. Compounds of the invention may also be formulated for transdermal administration as a transdermal patch.

Topical compositions are also provided comprising at least one chemical entity and a pharmaceutically acceptable vehicle well known in the art, such as, for example, water, alcohols, aloe vera gel, allantoin, glycerine, vitamin A and E oils, mineral oil, propylene glycol, and PPG-2 myristyl propionate.

Other materials suitable for use in topical carriers include, for example, emollients, solvents, humectants, thickeners and powders. Examples of each of these types of materials, which can be used singly or as mixtures of one or more materials, are as follows:

Emollients, such as stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, and myristyl myristate; propellants, such as propane, butane, iso-butane, dimethyl ether, carbon dioxide, and nitrous oxide; solvents, such as ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran; humectants, such as glycerin, sorbitol, sodium 2-pyrrolidone-5-carboxylate, soluble collagen, dibutyl phthalate, and gelatin; and powders, such as chalk, talc, fullers earth, kaolin, starch, gums, colloidal silicon dioxide, sodium polyacrylate, tetra alkyl ammonium smectites, trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, and ethylene glycol monostearate.

Compounds of the invention may also be topically administered in the form of liposome delivery systems, such as small unilamellar vesicles, large unilamellar vesicles, and multilamellar vesicles. Liposomes can be formed from a variety of phospholipids; such as cholesterol, stearylamine or phosphatidylcholines.

Other compositions useful for attaining systemic delivery of the subject compounds include sublingual, buccal and nasal dosage forms. Such compositions may comprise, for example, one or more of soluble filler substances such as sucrose, sorbitol and mannitol, and binders such as acacia, microcrystalline cellulose, carboxymethyl cellulose, and hydroxypropyl methylcellulose. Glidants, lubricants, sweeteners, colorants, antioxidants and flavoring agents disclosed above may also be included.

Compositions for inhalation can be provided, for example, in the form of a solution, suspension or emulsion that can be administered as a dry powder or in the form of an aerosol using, for example, a conventional propellant (e.g., dichlorodifluoromethane or trichlorofluoromethane).

The compositions of the present invention may also optionally comprise an activity enhancer. The activity enhancer can be chosen from a wide variety of molecules that function in different ways to enhance therapeutic effects of compounds of the invention. Particular classes of activity enhancers include skin penetration enhancers and absorption enhancers.

Pharmaceutical compositions of the invention may also contain additional active agents that can be chosen from a wide variety of molecules, which can function in different ways to enhance the therapeutic effects of a compound. These optional other active agents, when present, can be employed in the compositions of the invention at a level, for example, ranging from 0.01% to 15%. Some embodiments contain from 0.1% to 10% by weight of the composition. Other embodiments contain from 0.5% to 5% by weight of the composition.

Some embodiments of the invention includes packaged pharmaceutical formulations. Such packaged formulations include a pharmaceutical composition comprising at least one chemical entity provided herein in a container and instructions for using the composition to treat a mammal (for example a human patient). For instance, the instructions may be for using the pharmaceutical composition to treat a patient suffering from a disease responsive to inhibition of Btk activity and/or inhibition of B-cell proliferation. The invention includes providing prescribing information; for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the compounds of the invention can be administered alone, as mixtures, or in combination with other active agents.

Certain imidazo[1,2-a]pyrazines active as kinase inhibitors, in particular Btk inhibitors are described herein. These inhibitors can be useful for treating one or more diseases responsive to kinase inhibition, including diseases responsive to Btk inhibition and/or inhibition of B-cell proliferation, in mammals. Without wishing to be bound to any particular theory, it is believed that the interaction of the compounds of the invention with Btk results in the inhibition of Btk activity and thus in the pharmaceutical utility of these compounds. Other kinases that may be affected in addition to Btk include, but are not limited to, other tyrosine kinases and serine/threonine kinases.

Accordingly, the invention includes a method of treating a mammal, for instance a human, having a disease responsive to inhibition of Btk activity, comprising administrating to the mammal having such a disease, an effective amount of at least one chemical entity provided herein.

To the extent that Btk is implicated in any of the following, alleviation of the disease and/or disease symptoms and preventative and prophylactic treatment are within the scope of this invention. In addition, as noted above, the compounds of the invention may also inhibit other kinases, such that alleviation of disease, disease symptoms, preventative, and prophylactic treatment of conditions associated with these kinases is also within the scope of this invention.

Certain embodiments of the invention provide methods of treatment including inhibiting Btk activity and/or inhibiting B-cell proliferation, by inhibiting ATP binding or hydrolysis by Btk or by some other mechanism, in vivo, in a patient suffering from a disease responsive to inhibition of Btk activity, by administering an effective concentration of at least one chemical entity provided herein to inhibit Btk activity in vitro. An effective concentration may be ascertained experimentally, for example by assaying blood concentration of the compound, or theoretically, by calculating bioavailability.

Certain compounds described herein are useful for treating a patient suffering from a disease responsive to kinase inhibition.

Protein kinases, the largest family of human enzymes, are now considered to be the largest druggable target class. Encompassing well over 500 proteins (2% of the human genome), kinases play notable roles in signaling pathways controlling fundamental cellular processes such as proliferation, differentiation, and death (apoptosis). Abnormal kinase activity has been implicated in a wide range of diseases, including multiple cancers, autoimmune and/or inflammatory diseases, and acute inflammatory reactions. The multifaceted role of kinases in key cell signaling pathways provides a significant opportunity to identify novel drugs targeting kinases and signaling pathways.

Some embodiments include a method of treating a patient having cancer, an autoimmune and/or inflammatory disease, or an acute inflammatory reaction, by administering an effective amount of at least one chemical entity provided herein.

In some embodiments, the condition responsive to inhibition of Btk activity and/or B-cell proliferation is cancer, an autoimmune and/or inflammatory disease, or an acute inflammatory reaction.

The one or more conditions and diseases that can be affected using compounds and compositions according to the invention include, but are not limited to:

autoimmune and/or inflammatory diseases, including but not limited to psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis,

acute inflammatory reactions, including but not limited to skin sunburn, inflammatory pelvic disease, inflammatory bowel disease, urethritis, uvitis, sinusitis, pneumonitis, encephalitis, meningitis, myocarditis, nephritis, osteomyelitis, myositis, hepatitis, gastritis, enteritis, dermatitis, gingivitis, appendicitis, pancreatitis, and cholocystitis, and

cancer, including but not limited to, B-cell lymphoma, lymphoma (including Hodgkin's and non-Hodgkins lymphoma), hairy cell leukemia, multiple myeloma, chronic and acute myelogenous leukemia, and chronic and acute lymphocytic leukemia.

Btk is a known inhibitor of apoptosis in lymphoma B-cells. Defective apoptosis contributes to the pathogenesis and drug resistance of human leukemias and lymphomas. Thus, a method of promoting or inducing apoptosis in cells expressing Btk comprising contacting the cell with an agent that inhibits Btk activity is also provided herein.

Some embodiments provide methods of treatment in which at least one chemical entity provided herein is the only active agent given to a patient and also methods of treatment in which at least one chemical entity provided herein is given to a patient in combination with one or more additional active agents. Thus, in some embodiments, the invention provides a method of treating cancer, an autoimmune and/or inflammatory disease, or an acute inflammatory reaction, which comprises administering to a mammal in need thereof an effective amount of at least one chemical entity provided herein together with a second active agent, which is useful for treating an cancer, an autoimmune and/or inflammatory disease, or an acute inflammatory reaction. For example the second agent may be an anti-inflammatory agent. Treatment with the second active agent may be prior to, concomitant with, or following treatment with at least one chemical entity provided herein. In certain embodiments at least one chemical entity provided herein is combined with another active agent in a single dosage form. Suitable antitumor therapeutics that may be used in combination with at least one chemical entity provided herein include, but are not limited to chemotherapeutic agents, for example chosen from mitomycin C, carboplatin, taxol, cisplatin, paclitaxel, etoposide, and doxorubicin. Radiotherapeutic antitumor agents may also be used, alone or in combination with chemotherapeutic agents.

Btk inhibitors can be useful as chemosensitizing agents, and, thus, are useful in combination with other chemotherapeutic drugs, in particular, drugs that induce apoptosis.

A method for increasing sensitivity of cancer cells to chemotherapy, comprising administering to a patient undergoing chemotherapy a chemotherapeutic agent together with a Btk inhibitor in an amount sufficient to increase the sensitivity of cancer cells to the chemotherapeutic agent is also provided herein.

Examples of other chemotherapeutic drugs that can be used in combination with Btk inhibitors include topoisomerase I inhibitors (e.g. camptothesin and topotecan), topoisomerase II inhibitors (e.g. daunomycin and etoposide), alkylating agents (e.g. cyclophosphamide, melphalan and BCNU), tubulin directed agents (e.g. taxol and vinblastine), and biological agents (e.g. antibodies such as anti CD20 antibody, IDEC 8, immunotoxins, and cytokines).

Included herein are methods of treatment in which at least one chemical entity provided herein is administered in combination with an anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate.

Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors (i.e., a compound that inhibits COX-2 with an IC₅₀ that is at least 50-fold lower than the IC₅₀ for COX-1) such as celecoxib, valdecoxib, lumiracoxib, etoricoxib and/or rofecoxib.

In some embodiments, the anti-inflammatory agent is a salicylate. Salicylates include by are not limited to acetylsalicylic acid or aspirin, sodium salicylate, and choline and magnesium salicylates.

The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-C5 monoclonal antibody (such as eculizumab or pexelizumab), a TNF antagonist, such as entanercept, or infliximab, which is an anti-TNF alpha monoclonal antibody.

Still other embodiments of the invention pertain to combinations in which at least one active agent is an immunosuppressant compound such as an immunosuppressant compound chosen from methotrexate, leflunomide, cyclosporine, tacrolimus, azathioprine, and mycophenolate mofetil.

Dosage levels of the order of, for example, from 0.1 mg to 140 mg per kilogram of body weight per day, can be useful in the treatment of the above-indicated conditions (for instance 0.5 mg to 7 g per patient per day). The amount of active ingredient that may be combined with the carrier materials to produce a single dosage form will vary depending upon the host treated and the particular mode of administration. Dosage unit forms will generally contain from 1 mg to 500 mg of an active ingredient.

Frequency of dosage may also vary depending on the compound used and the particular disease treated. However, for treatment of most autoimmune and/or inflammatory diseases, a dosage regimen of 4 times daily or less is one example and a dosage regimen of 1 or 2 times daily is another example.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease in the patient undergoing therapy.

### EXAMPLES

### Example 1

### Exemplary Synthesis of Certain Imidazo[1,2-a]pyrazin-8-ylamines

### Step 1. 6,8-dibromoimidazo[1,2-a]pyrazine (3)

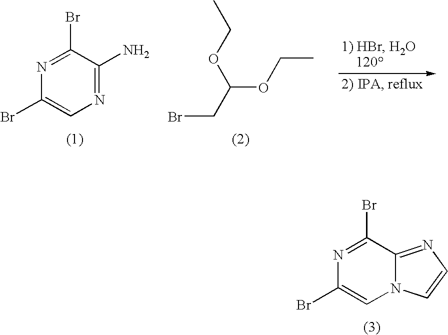

A mixture of bromoacetaldehyde diethyl acetal (51 grams (g)), 48% hydrobromic acid (HBr) (11 milliliters (mL)), and water (11 mL) is heated at 120° C. for 1 hour (hr). The solution is cooled, poured into a mixture of sodium bicarbonate (NaHCO₃) (60 g) and isopropyl alcohol (IPA) (200 mL), and stirred for 0.5 hr. The mixture is filtered, and the filtrate is treated with 3,5-dibromo-2-aminopyrazine (1) (33 g) and heated under reflux for 16 hr. The suspension is cooled in ice, treated with 48% HBr (3 mL) and diethyl ether (60 mL) and filtered to afford (3) as the hydrobromide salt.

### Step 2. (6-Bromo-imidazo[1,2-a]pyrazin-8-yl)-phenyl-amine (4)

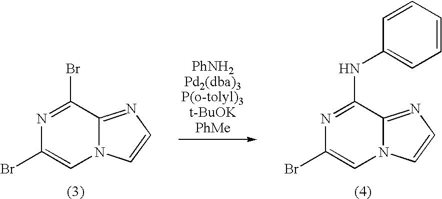

Tri-o-tolylphosphine (1.1 g) is added to a suspension of tris(dibenzylideneacetone)dipalladium (1.65 g) in degassed toluene (50 mL) and the mixture is stirred at room temperature for 15 minutes (min). 6,8-Dibromo-imidazo[1,2-a]pyrazine (5.0 g) is then added and the mixture is stirred at room temperature for 15 minutes more. Aniline (1.52 g) is added followed by potassium t-butoxide (2.84 g) and the mixture is stirred at room temperature for 72 hr. Water (150 mL) is added and the mixture is extracted with ethyl acetate (3×150 mL), the extracts are washed with water (1×100 mL) and brine (1×100 mL), dried over MgSO₄, and evaporated in vacuo to afford a gum. Trituration with diethyl ether affords (6-bromo-imidazo[1,2-a]pyrazin-8-yl)-phenyl-amine (4) as a brown solid.

### Step 3. [6-(3-Amino-phenyl)-imidazo[1,2-a]pyrazin-8-yl]-phenyl-amine (5)

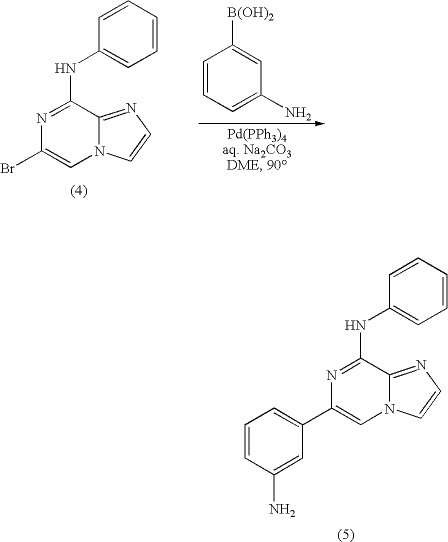

A mixture of (6-bromo-imidazo[1,2-a]pyrazin-8-yl)-phenyl-amine (4) (1.9 g), 3-aminophenylboronic acid hemisulfate (1.35 g), tetrakis(triphenylphosphine)palladium (380 mg), 1M aqueous sodium carbonate (20 mL), and 1,2-dimethoxyethane (40 mL) is heated at 90° for 16 hr. The mixture is cooled to room temperature, treated with water (50 mL) and extracted with ethyl acetate (3×80 mL). The extracts are washed with water (1×50 mL) and brine (1×50 mL), dried over MgSO₄, and evaporated in vacuo to afford a gum which is purified by flash chromatography over silica gel, eluting with ethyl acetate, to afford [6-(3-amino-phenyl)-imidazo[1,2-a]pyrazin-8-yl]-phenyl-amine (5) as a light brown solid.

### Step 4. 4-tert-Butyl-N-[3-(8-phenylamino-imidazo[1,2-a]pyrazin-6-yl)-phenyl]-benzamide (6)

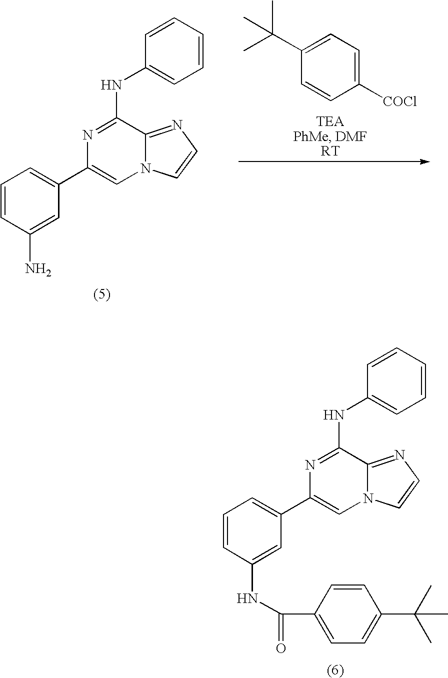

A solution of [6-(3-amino-phenyl)-imidazo[1,2-a]pyrazin-8-yl]-phenyl-amine (5) (50 mg), 4-tert-butyl-benzoyl chloride (0.83 mL of a 0.2M solution in toluene), and triethylamine (0.034 mL) in toluene (1 mL) and N,N,-dimethylformamide (1 mL) is stirred at room temperature for 16 hr. The mixture is purified by preparative thin layer chromatography over silica gel, eluting with diethyl ether/dichloromethane (1:1) to afford 4-tert-butyl-N-[3-(8-phenylamino-imidazo[1,2-a]pyrazin-6-yl)-phenyl]-benzamide (6) as a cream foam.

### Example 2

### Synthesis of N-(4-tert-Butyl-phenyl)-3-{8-[4-(morpholin-4-carbonyl)-phenylamino]-imidazo[1,2-a]pyrazin-6-yl}-benzamide

### Step. 1. 4-(6-Bromo-imidazo[1,2-a]pyrazin-8-ylamino)-benzoic acid (7)

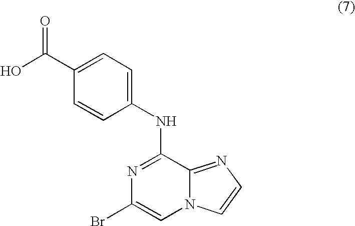

A mixture of 4-(6-Bromo-imidazo[1,2-a]pyrazin-8-ylamino)-benzoic acid ethyl ester (30 mmol), 1N aqueous sodium hydroxide (50 mL), and ethanol (80 mL) is heated at reflux for 1.5 hr. The mixture is cooled and the ethanol removed in vacuo. The residue is diluted with 1N sodium hydroxide (30 mL) and washed with ethyl acetate (2×100 mL). The aqueous layer is brought to pH 6 with 3N HCl, and the mixture filtered to give 4-(6-Bromo-imidazo[1,2-a]pyrazin-8-ylamino)-benzoic acid (7) as a light orange solid.

### Step 2. [4-(6-Bromo-imidazo[1,2-a]pyrazin-8-ylamino)-phenyl]-morpholin-4-yl-methanone (8)

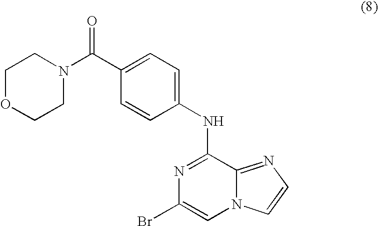

A mixture of 4-(6-Bromo-imidazo[1,2-a]pyrazin-8-ylamino)-benzoic acid (7) (0.16 mmol), benzotriazol-1-yloxy-tris(dimethylamino)-phosphonium hexafluorophosphate (0.16 mmol), and N′N-diisopropylethylamine (0.48 mmol) is stirred at room temperature for 20 min. under N₂. Morpholine (0.48 mmol) is added and the mixture is stirred at room temperature for 16 hr. Water (10 mL) is added and the mixture extracted with ethyl acetate (2×70 mL). The extracts are subsequently washed with water (2×30 mL) and brine (1×30 mL), dried over sodium sulfate, and concentrated in vacuo. The residue is triturated with diethyl ether to give [4-(6-Bromo-imidazo[1,2-a]pyrazin-8-ylamino)-phenyl]-morpholin-4-yl-methanone (8) as a cream solid.

### Step 3. 3-{8-[4-(Morpholine-4-carbonyl)-phenylamino]-imidazo[1,2-a]pyrazin-6-yl}-benzoic acid (9)

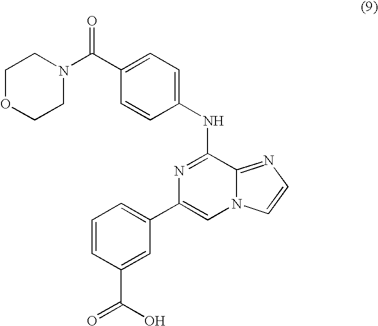

The bromide (8) (2.46 mmol) is dissolved in 10 mL DME (ethylene glycol, dimethyl ether) in a sealable pressure vessel and N₂ is bubbled through for 10 min. Tetrakis(triphenylphosphine)palladium (0.24 mmol) is added and N₂ is bubbled through the reaction for another 10 minutes. 3-Carboxyphenyl boronic acid (3.1 mmol) and 1N Na₂CO₃ solution (10 mL) are then added, the vessel sealed, and the reaction heated to 95° C. for 16 hrs. with vigorous stirring. After cooling to room temperature, the reaction is acidified to pH 3 with 1N HCl and extracted with ethyl acetate (3×25 mL). The pooled ethyl acetate layers are washed with brine, dried over sodium sulfate, and the solvent removed in vacuo. The resulting solid is then dissolved in a minimal volume of CH₂Cl₂ and precipitated out as a whitish-tan solid by slow addition of ethyl acetate (5 mL) followed by hexanes (30 mL) to yield 3-{8-[4-(Morpholine-4-carbonyl)-phenylamino]-imidazo[1,2-a]pyrazin-6-yl}-benzoic acid (9).

### Step 4. N-(4-tert-Butyl-phenyl)-3-{8-[4-(morpholine-4-carbonyl)-phenylamino]-imidazo[1,2-a]pyrazin-6-yl}-benzamide (10)

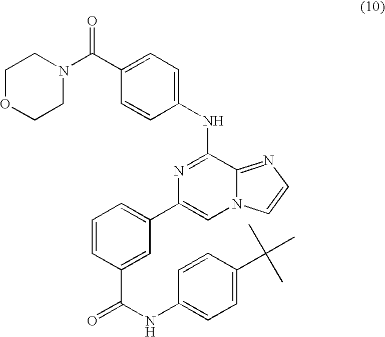

A mixture of 3-{8-[4-(Morpholine-4-carbonyl)-phenylamino]-imidazo[1,2-a]pyrazin-6-yl}-benzoic acid (9) (0.23 mmol), benzotriazol-1-yloxy-tris(dimethylamino)-phosphonium hexafluorophosphate reagent (0.23 mmol) and N,N′-diisopropylethylamine (0.77 mmol) is stirred under N₂ for 15 min. in 1 mL DMF. 4-tert-Butyl-phenylamine (0.37 mmol) is added and the reaction stirred at rt for 8 hrs. The reaction is partitioned between ethyl acetate (15 mL) and water (15 mL) and the ethyl acetate layer washed with water (2×15 mL) and brine (1×15 mL). The organic layer is dried over sodium sulfate and the solvent removed in vacuo. The resulting crude oil is dissolved in 1 ml CH₂Cl₂ and clean yellow solid is precipitated by slow addition of diethyl ether, yielding the final product, (10) N-(4-tert-Butyl-phenyl)-3-{8-[4-(morpholine-4-carbonyl)-phenylamino]-imidazo[1,2-a]pyrazin-6-yl}-benzamide.

### Example 3

### Synthesis of N-(3-{8-[4-(4-Acetyl-piperazin-1-yl)-phenylamino]-imidazo[1,2-a]pyrazin-6-yl}-phenyl)-4-tert-butyl-benzamide

### Step 1. 4-(4-Nitro-phenyl)-piperazine-1-carboxylic acid tert-butyl ester

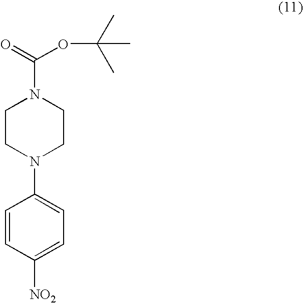

A mixture of 1-fluoro-4-nitrobenzene (20.4 mmol), piperazine-1-carboxylic acid tert-butyl ester (20.4 mmol), potassium carbonate (40.8 mmol), and N′N-dimethylformamide (80 mL) is heated at 60° C. for 3 hrs. The mixture is cooled to room temperature, treated with water (100 mL), extracted with ethyl acetate (3×80 mL). The extracts are washed with water (3×60 mL) and brine (1×75 ml), dried over magnesium sulfate, and concentrated in vacuo to give 4-(4-nitro-phenyl)-piperazine-1-carboxylic acid tert-butyl ester (11) as a yellow solid.

### Step 2. 4-(4-Amino-phenyl)-piperazine-1-carboxylic acid tert-butyl ester

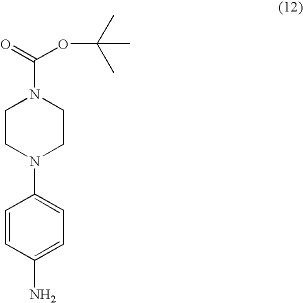

A mixture of 4-(4-nitro-phenyl)-piperazine-1-carboxylic acid tert-butyl ester (11) (18.9 mmol), 10% palladium-on-carbon (600 mg), ethanol (100 mL), and ethyl acetate (100 mL) is hydrogenated at room temperature and 40 psi for 2 hrs. The mixture is filtered through celite, washing with ethyl acetate (2×100 mL), and concentrated in vacuo to give 4-(4-amino-phenyl)-piperazine-1-carboxylic acid tert-butyl ester (12) as a brown oil.

### Step 3. 4-[4-(6-Bromo-imidazo[1,2-a]pyrazin-8-ylamino)-phenyl]-piperazine-1-carboxylic acid tert-butyl ester

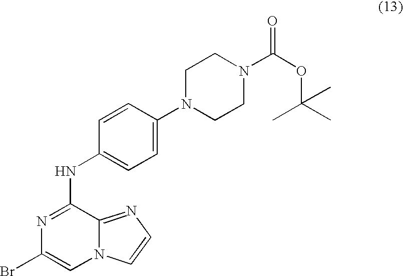

A mixture of 6,8-dibromoimidazo[1,2-a]pyrazine (12.5 mmol), 4-(4-amino-phenyl)-piperazine-1-carboxylic acid tert-butyl ester (12) (13.1 mmol), potassium carbonate (25 mmol), acetonitrile (50 mL) and N,N-dimethylacetamide (20 mL) is heated at 65° C. for 16 hrs. The mixture is cooled to room temperature, treated water (100 mL), and extracted with ethyl acetate (3×80 mL). The extracts are washed with water (3×60 mL) and brine (1×60 mL), dried over magnesium sulfate, and concentrated in vacuo. The residue is chromatographed over silica gel, eluting with ethyl acetate, to give 4-[4-(6-bromo-imidazo[1,2-a]pyrazin-8-ylamino)-phenyl]-piperazine-1-carboxylic acid tert-butyl ester (13) as a light brown foam.

### Step 4. 4-{4-[6-(3-Amino-phenyl)-imidazo[1,2-a]pyrazin-8-ylamino]-phenyl}-piperazine-1-carboxylic acid tert-butyl ester (14)

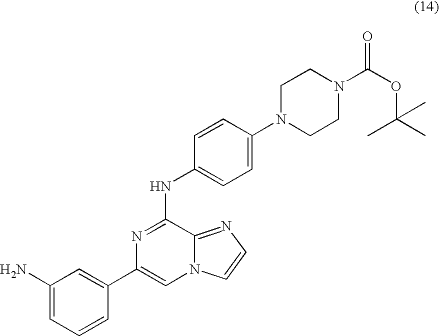

A mixture of 4-(6-bromoimidazo[1,2-a]pyrazin-8-ylamino)benzoic acid ethyl ester (13) (4.33 mmol), 3-aminophenylboronic acid hemisulfate (5.63 mmol), tetrakis(triphenylphosphine)palladium (0.2 mmol), 1N aqueous sodium carbonate solution (13 mL), and dimethoxyethane (70 mL) is heated at 95° C. for 2 days. The mixture is allowed to cool, treated with water (100 mL), and extracted with ethyl acetate (3×80 mL). The extracts are washed with water (2×75 mL) and brine (1×75 mL), dried over magnesium sulfate, and evaporated in vacuo. The residue is chromatographed over silica gel, eluting with ethyl acetate, to give 4-{4-[6-(3-amino-phenyl)-imidazo[1,2-a]pyrazin-8-ylamino]-phenyl}-piperazine-1-carboxylic acid tert-butyl ester (14) as a cream solid.

### Step 5. 4-(4-{6-[3-(4-tert-Butyl-benzoylamino)-phenyl]-imidazo[1,2-a]pyrazin-8-ylamino}-phenyl)-piperazine-1-carboxylic acid tert-butyl ester (15)

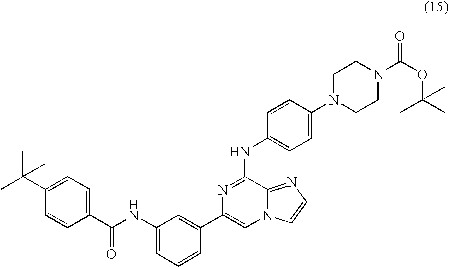

An ice-cold solution of 4-{4-[6-(3-Amino-phenyl)-imidazo[1,2-a]pyrazin-8-ylamino]-phenyl}-piperazine-1-carboxylic acid tert-butyl ester (14) (2.35 mmol), triethylamine (3.53 mmol), and tetrahydrofuran (20 mL) is treated dropwise with a solution of 4-t-butylbenzoyl chloride (2.35 mmol) in tetrahydrofuran (10 mL). The mixture is stirred at room temperature for 1 hr. Water (50 mL) is added and the mixture extracted with ethyl acetate (3×70 mL). The extracts are washed with water (2×50 mL) and brine (1×50 mL), dried over magnesium sulfate, and evaporated in vacuo. The residue is triturated with diethyl ether to give 4-(4-{6-[3-(4-tert-butyl-benzoylamino)-phenyl]-imidazo[1,2-a]pyrazin-8-ylamino}-phenyl)-piperazine-1-carboxylic acid tert-butyl ester (15) as a cream solid.

### Step 6. 4-tert-Butyl-N-{3-[8-(4-piperazin-1-yl-phenylamino)-imidazo[1,2-a]pyrazin-6-yl]-phenyl}-benzamide (16)

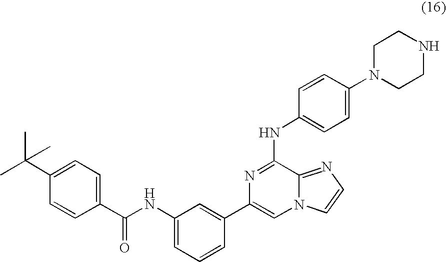

A mixture of 4-(4-{6-[3-(4-tert-butyl-benzoylamino)-phenyl]-imidazo[1,2-a]pyrazin-8-ylamino}-phenyl)-piperazine-1-carboxylic acid tert-butyl ester (15) (2.0 mmol), 1M HCl in diethyl ether (20 mL), and dioxane (60 mL) is stirred at room temperature for 16 hr. Water (100 mL) is added and the pH brought to 11 with 1N NaOH. The mixture is extracted with ethyl acetate (3×80 mL), the extracts washed with water (2×70 mL) and brine (1×70 mL), dried over magnesium sulfate, and concentrated in vacuo. The residue is slurried with diethyl ether and filtered to give 4-tert-butyl-N-{3-[8-(4-piperazin-1-yl-phenylamino)-imidazo[1,2-a]pyrazin-6-yl]-phenyl}-benzamide (16) as a cream solid.

### Step 7. N-(3-{8-[4-(4-Acetyl-piperazin-1-yl)-phenylamino]-imidazo[1,2-a]pyrazin-6-yl}-phenyl)-4-tert-butyl-benzamide (17)

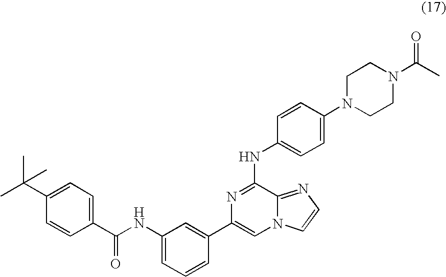

A mixture of 4-tert-butyl-N-{3-[8-(4-piperazin-1-yl-phenylamino)-imidazo[1,2-a]pyrazin-6-yl]-phenyl}-benzamide (16) (0.55 mmol), triethylamine (1.1 mmol), acetic anhydride (0.60 mmol), and dichloromethane (10 mL) is stirred at room temperature for 1 hr. Water (30 mL) is added, the mixture extracted with dichloromethane (2×50 mL), the extracts washed with water (2×30 mL) and brine (1×30 mL), dried over magnesium sulfate, and concentrated in vacuo. The residue is triturated with diethyl ether and ethyl acetate to give N-(3-{8-[4-(4-Acetyl-piperazin-1-yl)-phenylamino]-imidazo[1,2-a]pyrazin-6-yl}-phenyl)-4-tert-butyl-benzamide (17) as a cream solid.

### Example 4

### Synthesis of 4-Tert-butyl-N-(3-{8-[4-(morpholine-4-carbonyl)-phenylamino]-imidazo[1,2-a]pyrazin-6-yl}-phenyl)-benzamide

### Step 1. 4-(6-Bromo-imidazo[1,2-a]pyrazin-8-ylamino)-benzoic acid ethyl ester (18)

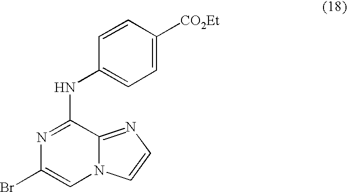

A mixture of 6,8-dibromoimidazo[1,2-a]pyrazine (0.18 mol), 4-aminobenzoic acid ethyl ester (2) (0.18 mol), and N,N-dimethylacetamide (30 mL) is heated at 160° C. for 30 min. The mixture is cooled to room temperature, treated with ethyl acetate (150 mL), stirred, and filtered to afford an orange/brown solid. This solid is slurried with ethyl acetate and saturated sodium bicarbonate solution to give 4-(6-bromoimidazo[1,2-a]pyrazin-8-ylamino)benzoic acid ethyl ester (18) freebase.

### Step 2. 4-[6-(3-Amino-phenyl)-imidazo[1,2-a]pyrazin-8-ylamino]-benzoic acid ethyl ester (19)

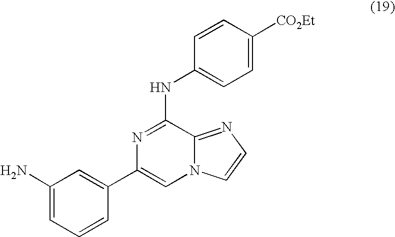

A mixture of 4-(6-bromoimidazo[1,2-a]pyrazin-8-ylamino)benzoic acid ethyl ester (18) (55.3 mmol), 3-aminophenylboronic acid hemisulfate (72 mmol), tetrakis(triphenylphosphine)palladium (2.8 mmol), 1N aqueous sodium carbonate solution (166 mmol), and dimethoxyethane (200 mL) is heated at reflux for 4 days. The mixture is allowed to cool, treated with water (200 mL), and extracted with ethyl acetate (4×100 mL). The extracts are washed with water (2×150 mL) and brine (1×100 mL), dried over magnesium sulfate, and evaporated in vacuo. The residue is triturated with dichloromethane and diethyl ether to give 4-[6-(3-aminophenyl)imidazo[1,2-a]pyrazin-8-ylamino]benzoic acid ethyl ester (19) as a yellow solid.

### Step 3. 4-{6-[3-(4-tert-Butyl-benzoylamino)-phenyl]-imidazo[1,2-a]pyrazin-8-ylamino}-benzoic acid ethyl ester (20)

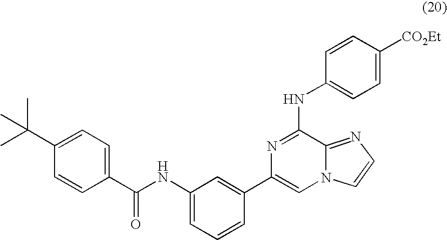

An ice-cold solution of 4-[6-(3-aminophenyl)imidazo[1,2-a]pyrazin-8-ylamino]benzoic acid ethyl ester (19) (44.2 mmol), triethylamine (66.4 mmol), and tetrahydrofuran (140 mL) is treated dropwise with a solution of 4-t-butylbenzoyl chloride (44.2 mmol) in tetrahydrofuran (60 mL) and the mixture is stirred at room temperature for 1 hr. Water (100 mL) is added and the mixture extracted with ethyl acetate (3×100 mL), the extracts are washed with water (2×100 mL) and brine (1×100 mL), dried over magnesium sulfate and evaporated in vacuo. The residue is triturated with dichloromethane to give 4-{6-[3-(4-tert-butyl-benzoylamino)phenyl]imidazo[1,2-a]pyrazin-8-ylamino}benzoic acid ethyl ester (20) as a pale orange solid.

### Step 4. 4-{6-[3-(4-tert-Butyl-benzoylamino)-phenyl]-imidazo[1,2-a]pyrazin-8-ylamino}-benzoic acid (21)

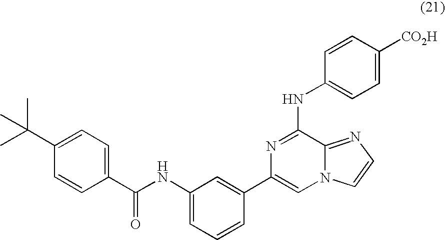

A mixture of 4-{6-[3-(4-tert-butyl-benzoylamino)phenyl]imidazo[1,2-a]pyrazin-8-ylamino}benzoic acid ethyl ester (20) (30 mmol), 1N aqueous sodium hydroxide (50 mL), and ethanol (80 mL) is heated at reflux for 1.5 hrs. The mixture is cooled and the ethanol removed in vacuo. The residue is diluted with 1N sodium hydroxide (30 mL) and washed with ethyl acetate (2×100 mL). The aqueous layer is brought to pH 6 with 3 N HCl, and the mixture is filtered to give 4-{6-[3-(4-tert-butyl-benzoylamino)phenyl]imidazo[1,2-a]pyrazin-8-ylamino}benzoic acid (21) as a cream solid.

### Step 5. 4-tert-Butyl-N-(3-{8-[4-(morpholine-4-carbonyl)-phenylamino]-imidazo[1,2-a]pyrazin-6-yl}-phenyl)-benzamide (22)

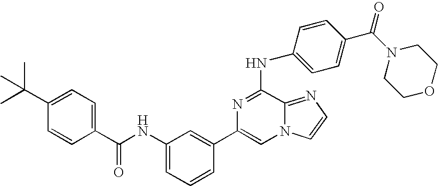

A mixture of 4-{6-[3-(4-tert-butyl-benzoylamino)phenyl]imidazo[1,2-a]pyrazin-8-ylamino}benzoic acid (21) (0.16 mmol), benzotriazol-1-yloxy-tris(dimethylamino)-phosphonium hexafluorophosphate (0.16 mmol), and N′N-diisopropylethylamine (0.48 mmol) is stirred at room temperature for 20 min. Morpholine (0.48 mmol) is added and the mixture is stirred at room temperature or 16 hrs. Water (10 mL) is added and the mixture extracted with ethyl acetate (2×70 mL), the extracts are washed with water (2×30 mL) and brine (1×30 mL), dried over magnesium sulfate, and concentrated in vacuo. The residue is triturated with diethyl ether to give 4-tert-B\butyl-N-(3-{8-[4-(morpholine-4-carbonyl)-phenylamino]imidazo[1,2-a]pyrazin-6-yl}-phenyl)benzamide (22) as a cream solid.

### Example 5

### Additional Imidazo[1,2-a]pyrazin-8-ylamines

The following compounds were prepared using procedures similar to those described above. In some instances changes in starting materials and reaction conditions that will be readily apparent to those skilled in the art of organic synthesis may be required.

LC-MS data reported in this example is obtained as follows:

LC conditions: RP-HPLC is performed on an AGILENT 1100 Binary HPLC system. The column is a Restek Ultra IBD 5 μm 1.0×30 mm (Cat. #: 9175331). The Mobile Phase contains component A, 0.2% Formic Acid/Water), and component B, Acetonitrile. The following Gradient is used:

| Time | | Flow Rate |
|---|---|---|
| (min.) | % B | (μl/min) |
| 0 | 10 | 500 |
| 1.8 | 60 | 500 |
| 2.0 | 95 | 500 |
| 2.2 | 95 | 500 |
| 2.4 | 10 | 500 |

MS conditions: Electrospray MS is performed on a MICROMASS LCT equipped with a LockSpray source for accurate mass measurements. Spectra are acquired in positive ion mode from 100-1000 Da at an acquisition rate of 1 spectrum/0.9 s with a 0.1 s interscan delay. The instrument is tuned for a resolution of 5000 (FWHM). Every 5^{th} scan is taken from the reference position of the Lockspray source. Leucine enkephalin (556.2771 [M+H]⁺) is used as the reference, or lock mass.

TABLE I

| Cmp. | | Name and | | MS |
|---|---|---|---|---|
| # | Structure | Molecular Formula | MW | m/z (M⁺ 1) |
| 23 | 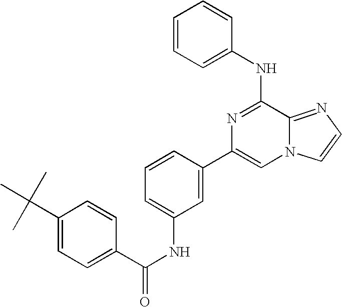 | 4-tert-Butyl-N-[3-(8- phenylamino- imidazo[1,2-a]pyrazin- 6-yl)-phenyl]- benzamide MF = C₂₉H₂₇N₅O | 461.22 | 462.21 |
| 24 | 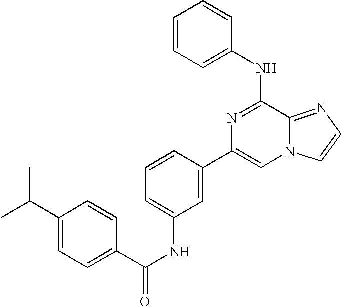 | 4-Isopropyl-N-[3-(8- phenylamino- imidazo[1,2-a]pyrazin- 6-yl)-phenyl]- benzamide MF = C₂₈H₂₅N₅O | 447.21 | 448.21 |
| 25 | 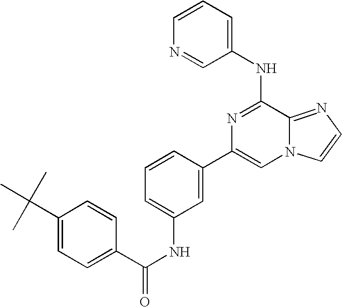 | 4-tert-Butyl-N-{3-[8- (pyridin-3-ylamino)- imidazo[1,2-a]pyrazin- 6-yl]-phenyl}- benzamide MF = C₂₈H₂₆N₆O | 462.21 | 463.07 |
| 26 | 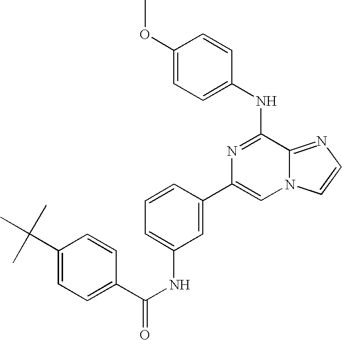 | 4-tert-Butyl-N-{3-[8- (4-methoxy- phenylamino)- imidazo[1,2-a]pyrazin- 6-yl]-phenyl}- benzamide MF = C₃₀H₂₉N₅O₂ | 491.23 | 492.10 |
| 27 | 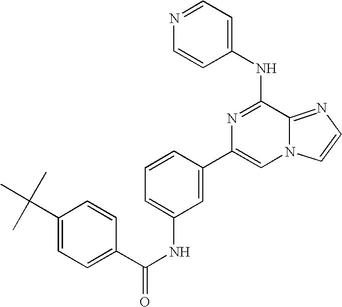 | 4-tert-Butyl-N-{3-[8- (pyridin-4-ylamino)- imidazo[1,2-a]pyrazin- 6-yl]-phenyl}- benzamide MF = C₂₈H₂₆N₆O | 462.22 | 463.19 |
| 28 | 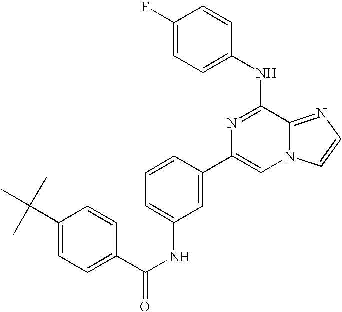 | 4-tert-Butyl-N-{3-[8- (4-fluoro- phenylamino)- imidazo[1,2-a]pyrazin- 6-yl]-phenyl}- benzamide MF = C₂₉H₂₆FN₅O | 479.21 | 480.10 |
| 29 | 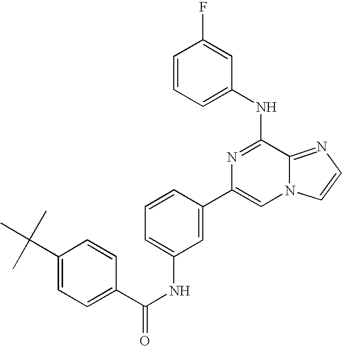 | 4-tert-Butyl-N-{3-[8- (3-fluoro- phenylamino)- imidazo[1,2-a]pyrazin- 6-yl]-phenyl}- benzamide MF = C₂₉H₂₆FN₅O | 479.21 | 480.10 |
| 30 | 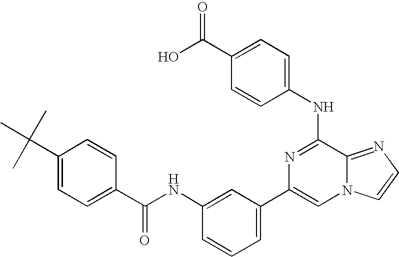 | 4-{6-[3-(4-tert-Butyl- benzoylamino)-phenyl]- imidazo[1,2-a]pyrazin- 8-ylamino}-benzoic acid MF = C₃₀H₂₇N₅O₃ | 505.21 | 506.08 |
| 31 | 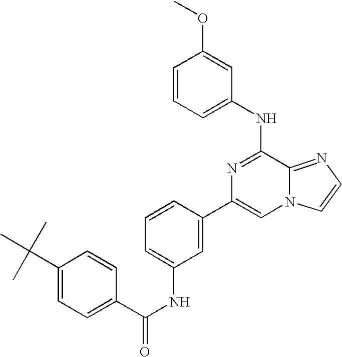 | 4-tert-Butyl-N-{3-[8- (3-methoxy- phenylamino)- imidazo[1,2-a]pyrazin- 6-yl]-phenyl}- benzamide MF = C₃₀H₂₉N₅O₂ | 491.23 | 492.08 |
| 32 | 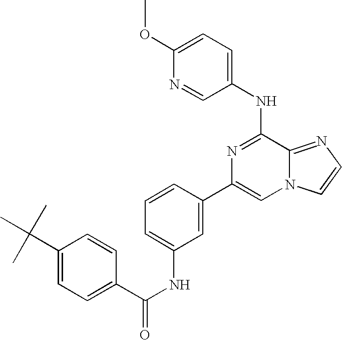 | 4-tert-Butyl-N-{3-[8- (6-methoxy-pyridin-3- ylamino)-imidazo[1,2- a]pyrazin-6-yl]- phenyl}-benzamide MF = C₂₉H₂₈N₆O₂ | 492.22 | 493.08 |
| 33 | 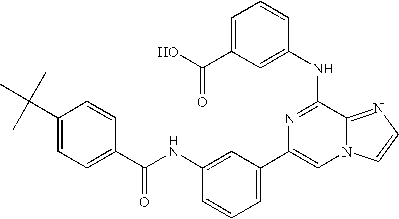 | 3-{6-[3-(4-tert-Butyl- benzoylamino)-phenyl]- imidazo[1,2-a]pyrazin- 8-ylamino}-benzoic acid MF = C₃₀H₂₇N₅O₃ | 505.21 | 506.25 |
| 34 | 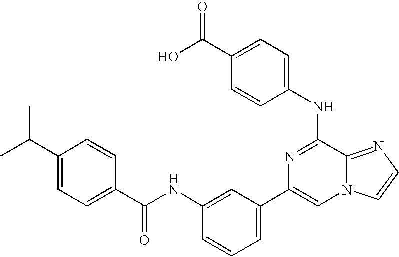 | 4-{6-[3-(4-Isopropyl- benzoylamino)-phenyl]- imidazo[1,2-a]pyrazin- 8-ylamino}-benzoic acid MF = C₂₉H₂₅N₅O₃ | 491.19 | 492.19 |
| 35 | 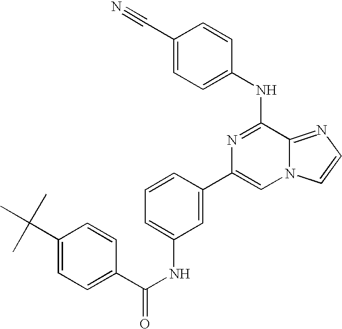 | 4-tert-Butyl-N-{3-[8- (4-cyano-phenylamino)- imidazo[1,2-a]pyrazin- 6-yl]-phenyl}- benzamide MF = C₃₀H₂₆N₆O | 486.22 | 487.28 |
| 36 | 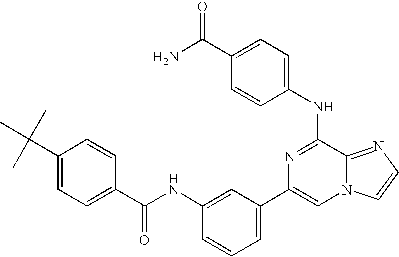 | 4-{6-[3-(4-tert-Butyl- benzoylamino)-phenyl]- imidazo[1,2-a]pyrazin- 8-ylamino}-benzamide MF = C₃₀H₂₈N₆O₂ | 504.22 | 505.21 |

TABLE II

| | | | | MS m/z |
|---|---|---|---|---|
| Cmp. # | STRUCTURE | NAME | MW | (M⁺¹) |
| 37 | 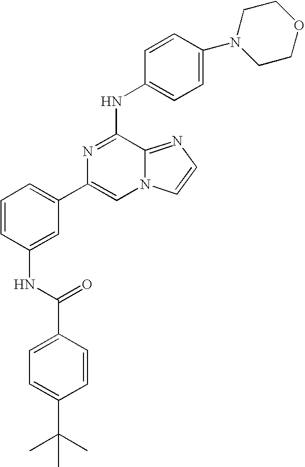 | 4-tert-Butyl-N-{3-{8-(4- morpholin-4-yl- phenylamino)-imidazo[1,2- a]pyrazin-6-yl]-phenyl)- benzamide C33H34N6O2 | 546.27 | 547.11 |
| 38 | 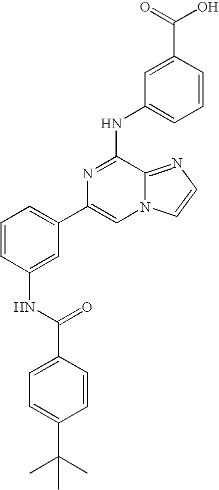 | 3-{6-[3-(4-tert-Butyl- benzoylamino)-phenyl]- imidazo[1,2-a]pyrazin-8- ylamino}-benzoic acid C30H27N5O3 | 505.21 | 506.25 |
| 39 | 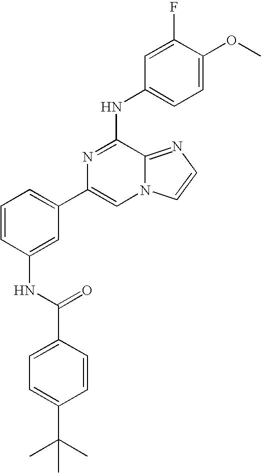 | 4-tert-Butyl-N-{3-[8-(3- fluoro-4-methoxy- phenylamino)-imidazo[1,2- a]pyrazin-6-yl]-phenyl}- benzamide C30H28FN5O2 | 509.22 | 510.26 |
| 40 | 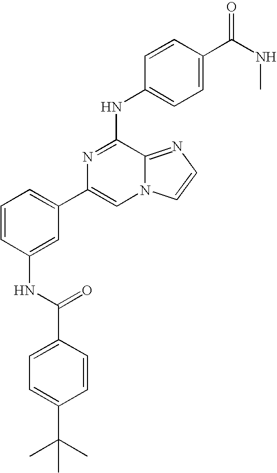 | 4-{6-[3-(4-tert-Butyl- benzoylamino)-phenyl]- imidazo[1,2-a]pyrazin-8- ylamino}-benzoic N-Methyl amide C31H30N6O2 | 518.24 | 519.21 |
| 41 | 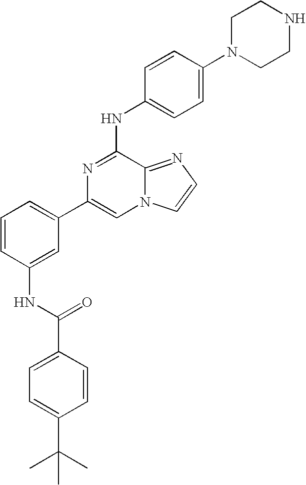 | 4-tert-Butyl-N-{3-[8-(4- piperazin-1-yl- phenylamino)-imidazo[1,2- a]pyrazin-6-yl]-phenyl}- benzamide C33H35N7O | 545.29 | 546.19 |
| 42 | 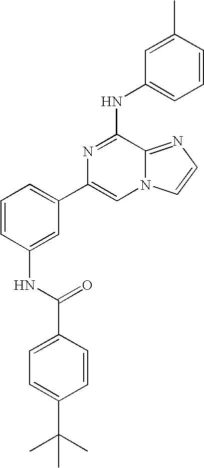 | 4-tert-Butyl-N-{3-(8-m- tolylamino-imidazo[1,2- a]pyrazin-6-yl)-phenyl]- benzamide C30H29N5O | 475.23 | 476.22 |
| 43 | 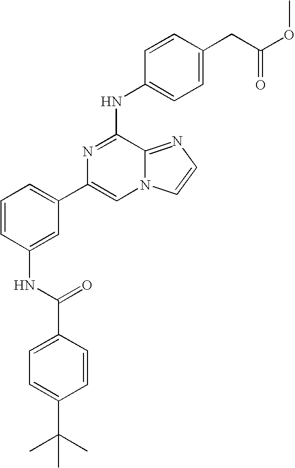 | (4-{6-[3-)4-tert-Butyl- benzoylamino)-phenyl]- imidazo[1,2-a]pyrazin-8- ylamino}-phenyl)-acetic acid methyl ester C32H31N5O3 | 533.24 | 534.22 |
| 44 | 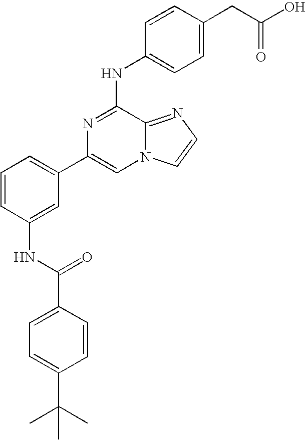 | (4-{6-[3-(4-tert-Butyl- benzoylamino)-phenyl]- imidazo[1,2-a]pyrazin-8- ylamino}-phenyl)-acetic acid C31H29N5O3 | 519.22 | 520.22 |
| 45 | 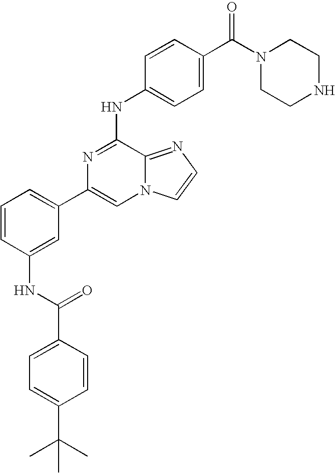 | 4-tert-Butyl-N-(3-{8-[4- (piperazine-1-carbonyl)- phenylamino]-imidazo[1,2- a]pyrazin-6-yl}-phenyl)- benzamide C34H35N7O2 | 573.28 | 574.85 |
| 46 | 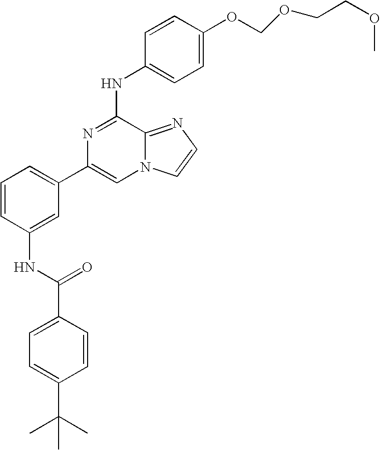 | 4-tert-Butyl-N-(3-{8-[4-(2- methoxy-ethoxymethoxy)- phenylamino]-imidazo[1,2- a]pyrazin-6-yl}-phenyl)- benzamide C33H35N5O4 | 565.26 | 566.25 |
| 47 | 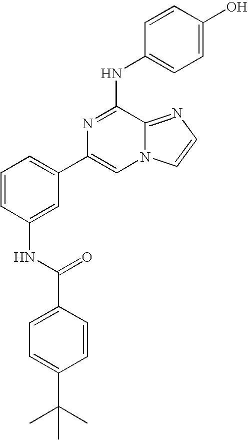 | 4-tert-Butyl-N-{3-[8-(4- hydroxy-phenylamino)- imidazo[1,2-a]pyrazin-6-yl]- phenyl}-benzamide C29H27N5O2 | 477.22 | 478.22 |
| 48 | 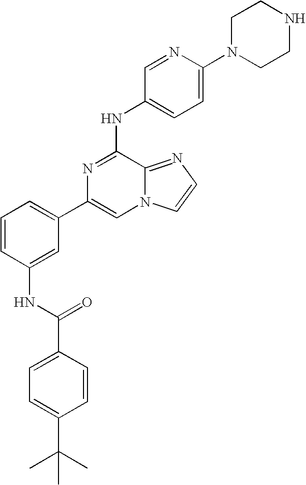 | 4-tert-Butyl-N-{3-[8-(6- piperazin-1-yl-pyridin-3- ylamino)-imidazo[1,2- a]pyrazin-6-yl]-phenyl}- benzamide C32H34N8O | 546.28 | 547.24 |
| 49 | 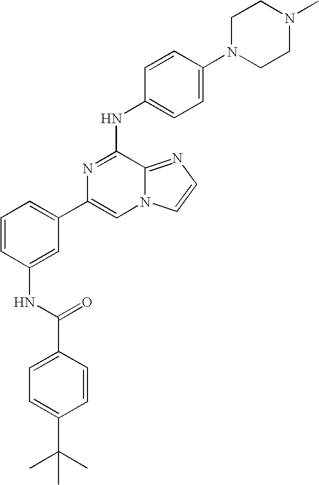 | 4-tert-Butyl-N-(3-{8-[4-(4- methyl-piperazin-1-yl)- phenylamino]-imidazo[1,2- a]pyrazin-6-yl}-phenyl)- benzamide C34H37N7O | 559.31 | 560.26 |
| 50 | 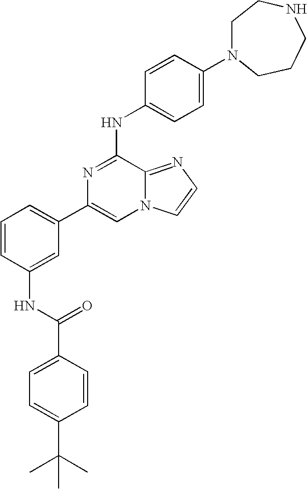 | 4-tert-Butyl-N-{3-[8-(4- [1,4]diazepan-1-yl- phenylamino)-imidazo[1,2- a]pyrazin-6-yl]-phenyl}- benzamide C34H37N7O | 559.31 | 561.17 |
| 51 | 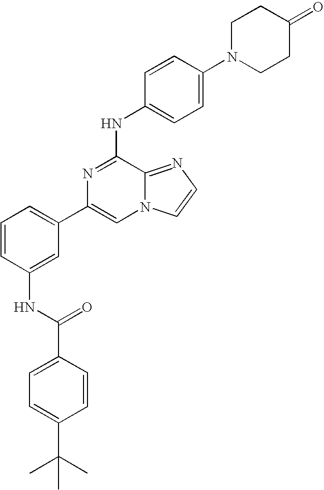 | 4-tert-Butyl-N-(3-{8-[4-(4- oxo-piperidin-1-yl)- phenylamino]-imidazo[1,2- a]pyrazin-6-yl}-phenyl)- benzamide C34H34N6O2 | 558.27 | 559.22 |
| 52 | 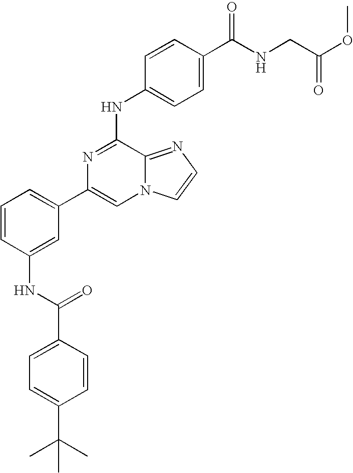 | (4-{6-[3-(4-tert-Butyl- benzoylamino)-phenyl]- imidazo[1,2-a]pyrazin-8- ylamino}-benzoylamino)- acetic acid methyl ester C33H32N6O4 | 576.24 | 577.29 |
| 53 | 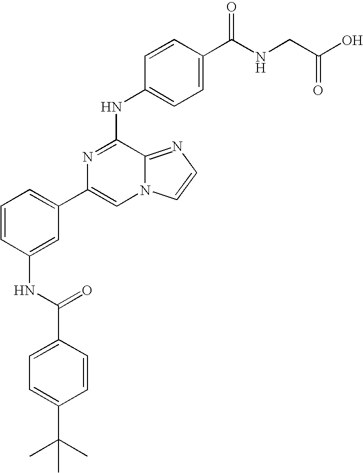 | (4-{6-[3-(4-tert-Butyl- benzoylamino)-phenyl]- imidazo[1,2-a]pyrazin-8- ylamino}-benzoylamino)- acetic acid C32H30N6O4 | 562.23 | 563.26 |
| 54 | 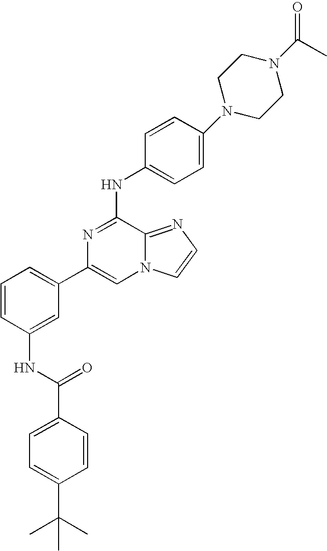 | N-(3-{8-[4-(4-Acetyl- piperazin-1-yl)- phenylamino]-imidazo[1,2- a]pyrazin-6-yl}-phenyl)-4- tert-butyl-benzamide C35H37N7O2 | 587.3 | 588.31 |
| 55 | 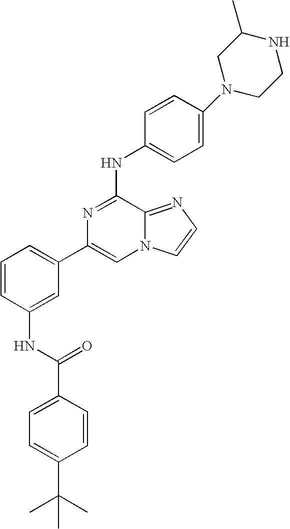 | 4-tert-Butyl-N-(3-{8-[4-(3- methyl-piperazin-1-yl)- phenylamino]-imidazo[1,2- a]pyrazin-6-yl}-phenyl)- benzamide C34H37N7O | 559.31 | 560.35 |
| 56 | 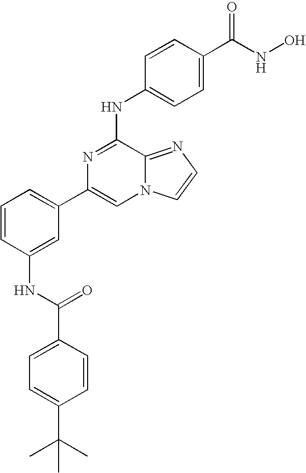 | 4-{6-[3-(4-tert-Butyl- benzoylamino)-phenyl]- imidazo[1,2-a]pyrazin-8- ylamino}-benzoic hydroxyl amide C30H28N6O3 | 520.22 | 521.26 |
| 57 | 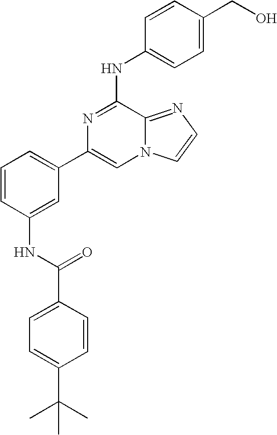 | 4-tert-Butyl-N-{3-[8-(4- hydroxymethyl- phenylamino)-imidazo[1,2- a]pyrazin-6-yl]-phenyl)- benzamide C30H29N5O2 | 491.23 | 492.28 |
| 58 | 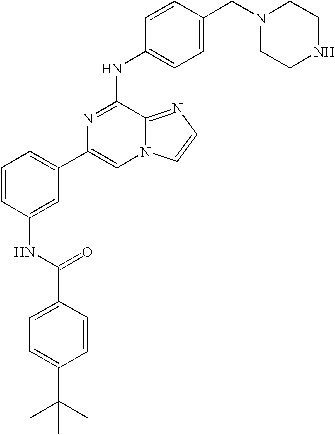 | 4-tert-Butyl-N-{3-[8-(4- piperazin-1-ylmethyl- phenylamino)-imidazo[1,2- a]pyrazin-6-yl]-phenyl}- benzamide C34H37N7O | 559.31 | 560.37 |
| 59 | 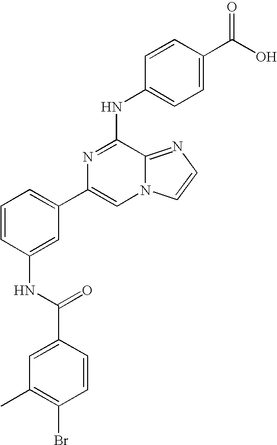 | 4-{6-[3-(4-Bromo-3-methyl- benzoylamino)-phenyl]- imidazo[1,2-a]pyrazin-8- ylamino}-benzoic acid C27H20BrN5O3 | 541.07 | 542.14 |
| 60 | 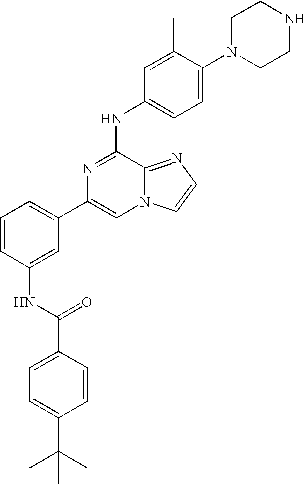 | 4-tert-Butyl-N-{3-[8-(3- methyl-4-piperazin-1-yl- phenylamino)-imidazo[1,2- a]pyrazin-6-yl]-phenyl}- benzamide C34H37N7O | 559.31 | 560.35 |
| 61 | 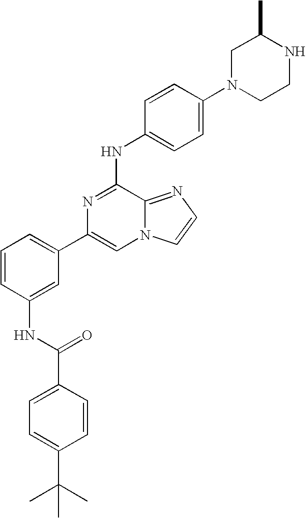 | 4-tert-Butyl-N-(3-{8-[4-(3- methyl-piperazin-1-yl)- phenylamino]-imidazo[1,2- a]pyrazin-6-yl}-phenyl)- benzamide C34H37N7O | 559.31 | 560.26 |
| 62 | 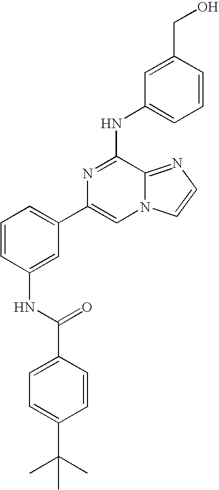 | 4-tert-Butyl-N-{3-[8-(3- hydroxymethyl- phenylamino)-imidazo[1,2- a]pyrazin-6-yl]-phenyl}- benzamide C30H29N5O2 | 491.23 | 492.28 |
| 63 | 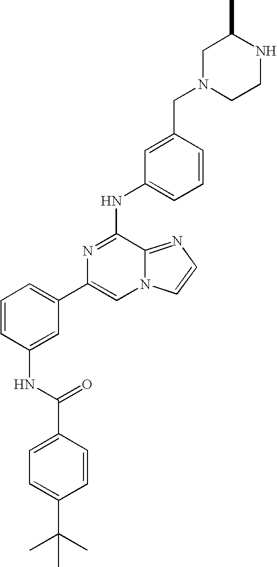 | 4-tert-Butyl-N-(3-{8-[3-(3- methyl-piperazin-1- ylmethyl)-phenylamino]- imidazo[1,2-a]pyrazin-6-yl}- phenyl)-benzamide C35H39N7O | 573.32 | 574.4 |
| 64 | 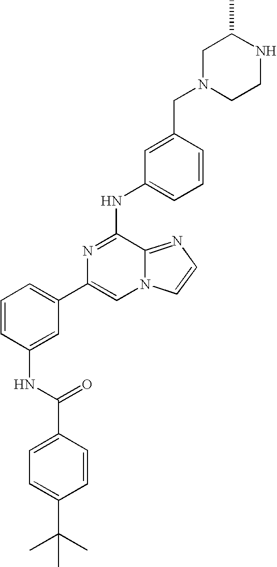 | 4-tert-Butyl-N-(3-{8-[3-(3- methyl-piperazin-1- ylmethyl)-phenylamino]- imidazo[1,2-a]pyrazin-6-yl}- phenyl)-benzamide C35H39N7O | 573.32 | 574.35 |
| 65 | 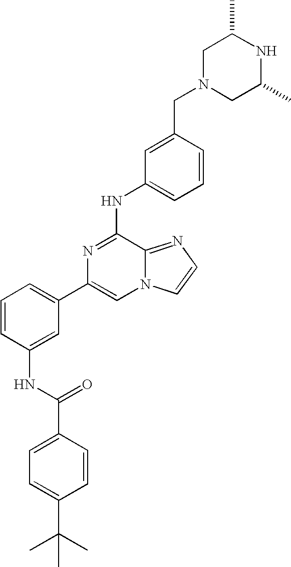 | 4-tert-Butyl-N-(3-{8-[3-(3,5- dimethyl-piperazin-1- ylmethyl)-phenylamino]- imidazo[1,2-a]pyrazin- 6-yl}-phenyl)-benzamide C36H41N7O | 587.33 | 588.39 |
| 66 | 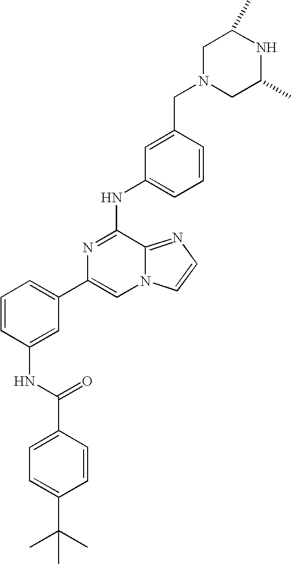 | 4-tert-Butyl-N-(3-{8-[4-(3,5- dimethyl-piperazin-1-yl)- phenylamino]-imidazo[1,2- a]pyrazin-6-yl}- phenyl)-benzamide C35H39N7O | 573.32 | 574.42 |
| 67 | 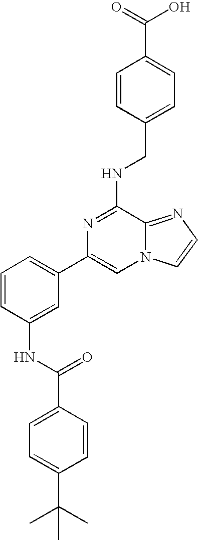 | 4-({6-[3-(4-tert-Butyl- benzoylamino)-phenyl]- imidazo[1,2-a]pyrazin-8- ylamino}-methyl)-benzoic acid C31H29N5O3 | 519.22 | 520.4 |
| 68 | 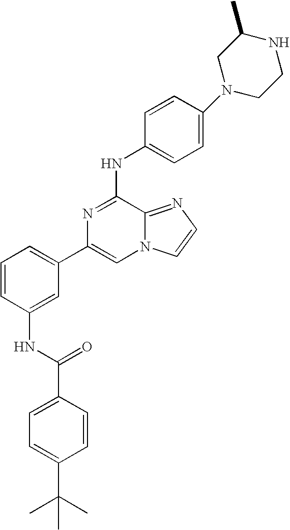 | 4-tert-Butyl-N-(3-{8-[4-(3- methyl-piperazin-1-yl)- phenylamino]-imidazo[1,2- a]pyrazin-6-yl}-phenyl)- benzamide C34H37N7O | 559.3 | 560.47 |
| 69 | 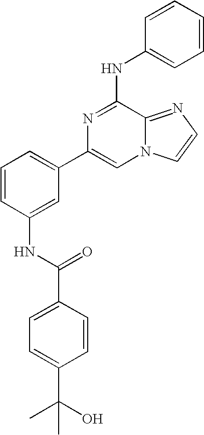 | 4-(1-Hydroxy-1-methyl- ethyl)-N-[3-(8-phenylamino- imidazo[1,2-a]pyrazin-6-yl)- phenyl]-benzamide C28H25N5O2 | 463.2 | 464.36 |
| 70 | 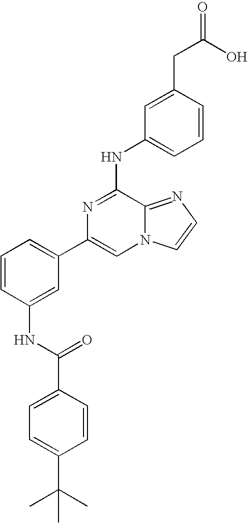 | (3-{6-[3-(4-tert-Butyl- benzoylamino)-phenyl]- imidazo[1,2-a]pyrazin-8- ylamino}-phenyl)-acetic acid C31H29N5O3 | 519.22 | 520.4 |
| 71 | 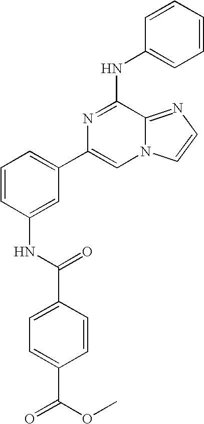 | N-[3-(8-Phenylamino- imidazo[1,2-a]pyrazin-6-yl)- phenyl]-terephthalamic acid methyl ester C27H21N5O3 | 463.16 | 464.32 |
| 72 | 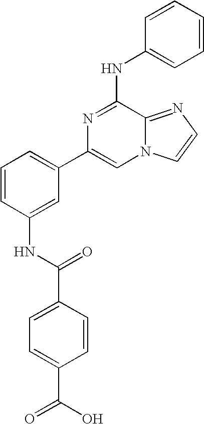 | N-[3-(8-Phenylamino- imidazo[1,2-a]pyrazin-6-yl)- phenyl]-terephthalamic acid C26H19N5O3 | 449.14 | 450.36 |
| 73 | 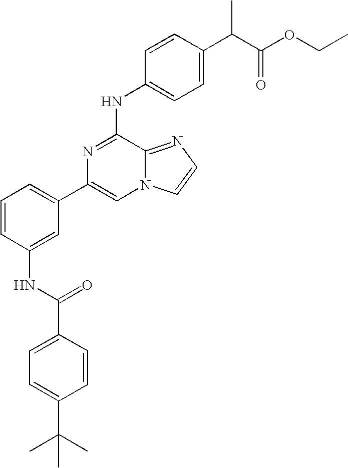 | 2-(4-{6-[3-(4-tert-Butyl- benzoylamino)-phenyl]- imidazo[1,2-a]pyrazin-8- ylamino}-phenyl)-propionic acid ethyl ester C34H35N5O3 | 561.27 | 562.45 |
| 74 | 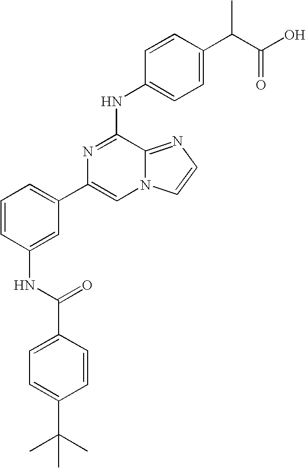 | 2-(4-{6-[3-(4-tert-Butyl- benzoylamino)-phenyl]- imidazo[1,2-a]pyrazin-8- ylamino}-phenyl)-propionic acid C32H31N5O3 | 533.24 | 534.43 |
| 75 | 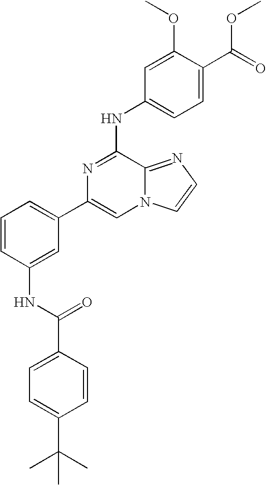 | 4-{6-[3-(4-tert-Butyl- benzoylamino)-phenyl]- imidazo[1,2-a]pyrazin-8- ylamino}-2-methoxy- benzoic acid methyl ester C32H31N5O4 | 549.23 | 550.42 |
| 76 | 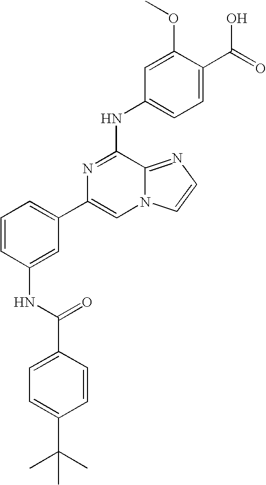 | 4-{6-[3-(4-tert-Butyl- benzoylamino)-phenyl]- imidazo[1,2-a]pyrazin-8- ylamino}-2-methoxy- benzoic acid C31H29N5O4 | 535.22 | 536.42 |
| 77 | 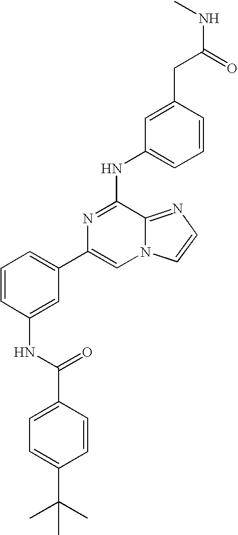 | 4-tert-Butyl-N-{3-[8-(3- methylcarbamoylmethyl- phenylamino)-imidazo[1,2- a]pyrazin-6-yl]-phenyl}- benzamide C32H32N6O2 | 532.26 | 533.4 |
| 78 | 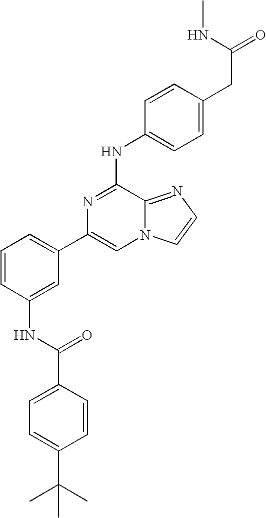 | 4-tert-Butyl-N-{3-[8-(4- methylcarbamoylmethyl- phenylamino)-imidazo[1,2- a]pyrazin-6-yl]-phenyl}- benzamide C32H32N6O2 | 532.25 | 533.41 |
| 79 | 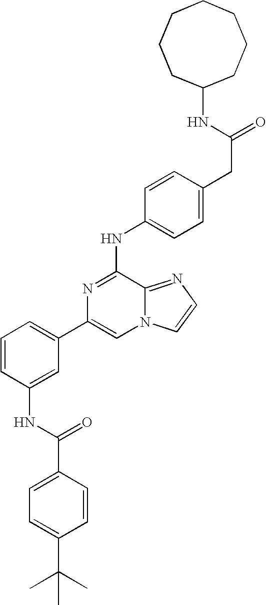 | 4-tert-Butyl-N-{3-[8-(4- cyclooctylcarbamoylmethyl- phenylamino)-imidazo[1,2- a]pyrazin-6-yl]-phenyl}- benzamide C39H44N6O2 | 628.35 | 629.51 |
| 80 | 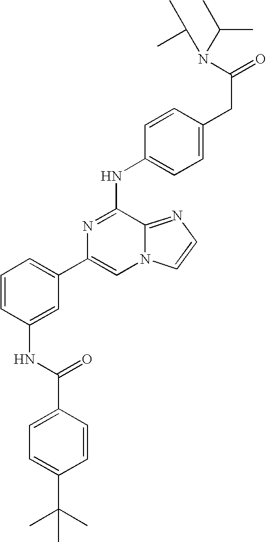 | 4-tert-Butyl-N-[3-(8-{4- [(diisopropylcarbamoyl)- methyl]-phenylamino}- imidazo[1,2-a]pyrazin-6-yl)- phenyl]-benzamide C37H42N6O2 | 602.34 | 603.49 |
| 81 | 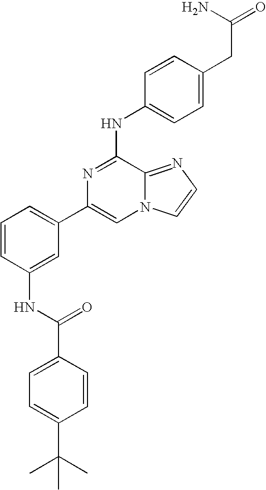 | 4-tert-Butyl-N-{3-[8-(4- carbamoylmethyl- phenylamino)-imidazo[1,2- a]pyrazin-6-yl]-phenyl}- benzamide C31H30N6O2 | 518.24 | 519.48 |
| 82 | 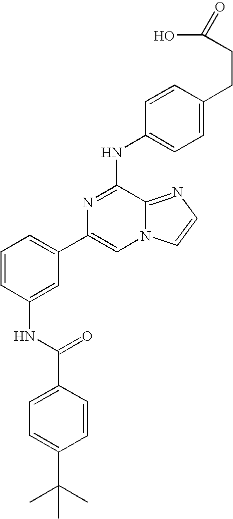 | 3-(4-{6-[3-(4-tert-Butyl- benzoylamino)-phenyl]- imidazo[1,2-a]pyrazin-8- ylamino}-phenyl)-propionic acid C32H31N5O3 | 533.24 | 534.22 |
| 83 | 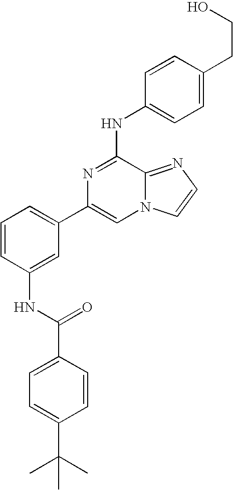 | 4-tert-Butyl-N-(3-{8-[4-(2- hydroxy-ethyl)- phenylamino]-imidazo[1,2- a]pyrazin-6-yl}-phenyl)- benzamide C31H31N5O2 | 505.25 | 506.4 |
| 84 | 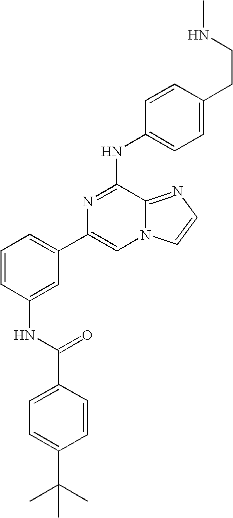 | 4-tert-Butyl-N-(3-{8-[4-(2- methylamino-ethyl)- phenylamino]-imidazo[1,2- a]pyrazin-6-yl}-phenyl)- benzamide C32H34N6O | 518.3 | 519.45 |
| 85 | 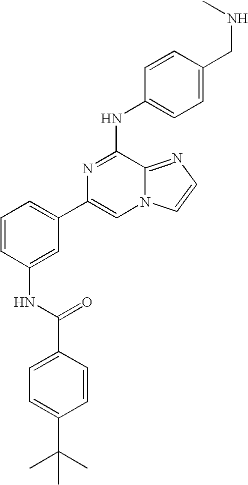 | 4-tert-Butyl-N-{3-[8-(4- methylaminomethyl- phenylamino)-imidazo[1,2- a]pyrazin-6-yl]-phenyl}- benzamide C31H32N6O | 504.26 | 505.43 |
| 86 | 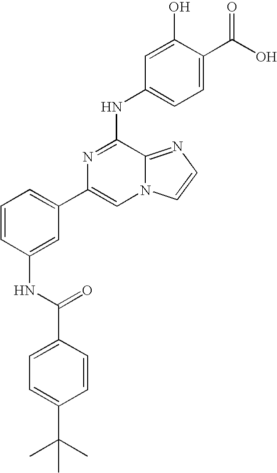 | 4-{6-[3-(4-tert-Butyl- benzoylamino)-phenyl]- imidazo[1,2-a]pyrazin-8- ylamino}-2-hydroxy-benzoic acid C30H27N5O4 | 521.2 | 522.31 |
| 87 | 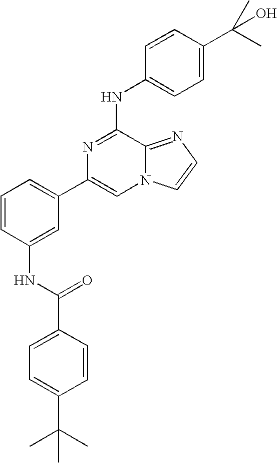 | 4-tert-Butyl-N-(3-{8-[4-(1- hydroxy-1-methyl-ethyl)- phenylamino]-imidazo[1,2- a]pyrazin-6-yl}-phenyl)- benzamide C32H33N5O2 | 519.26 | 520.27 |
| 88 | 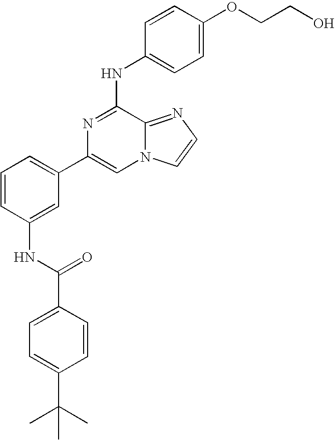 | 4-tert-Butyl-N-(3-{8-[4-(2- hydroxy-ethoxy)- phenylamino]-imidazo[1,2- a]pyrazin-6-yl}-phenyl)- benzamide C31H31N5O3 | 521.24 | 522.25 |
| 89 | 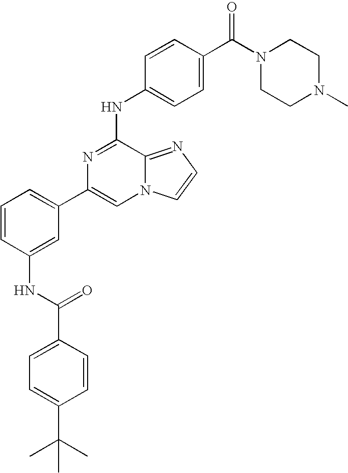 | 4-tert-Butyl-N-(3-{8-[4-(4- methyl-piperazine-1- carbonyl)-phenylamino]- imidazo[1,2-a]pyrazin-6-yl}- phenyl)-benzamide C35H37N7O2 | 587.3 | 588.28 |
| 90 | 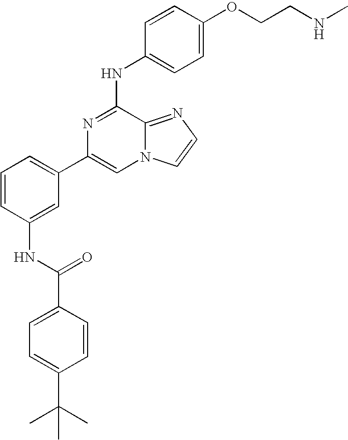 | 4-tert-Butyl-N-(3-{8-[4-(2- methylamino-ethoxy)- phenylamino]-imidazo[1,2- a]pyrazin-6-yl}-phenyl)- benzamide C32H34N6O2 | 534.27 | 535.27 |
| 91 | 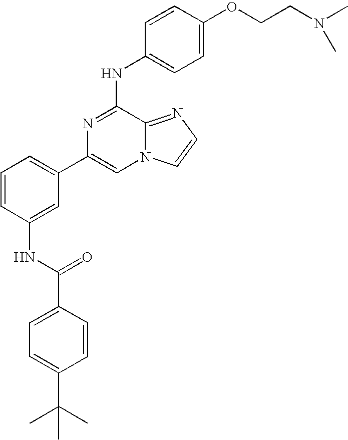 | 4-tert-Butyl-N-(3-{8-[4-(2- dimethylamino-ethoxy)- phenylamino]-imidazo[1,2- a]pyrazin-6-yl}-phenyl)- benzamide C33H36N6O2 | 548.28 | 549.28 |
| 92 | 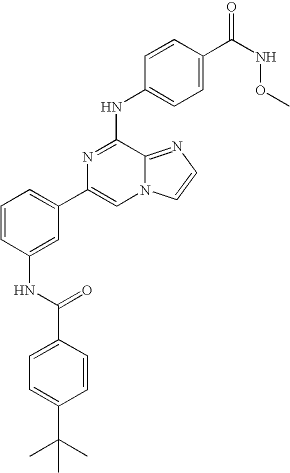 | 4-{6-[3-(4-tert-Butyl- benzoylamino)-phenyl]- imidazo[1,2-a]pyrazin-8- ylamino}-benzoic methoxy amide C31H30N6O3 | 534.23 | 535.25 |
| 93 | 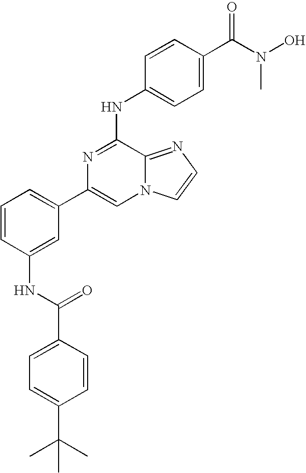 | 4-{6-[3-(4-tert-Butyl- benzoylamino)-phenyl]- imidazo[1,2-a]pyrazin-8- ylamino}-benzoic hyrdoxyl methyl amide C31H30N6O3 | 534.23 | 535.25 |
| 94 | 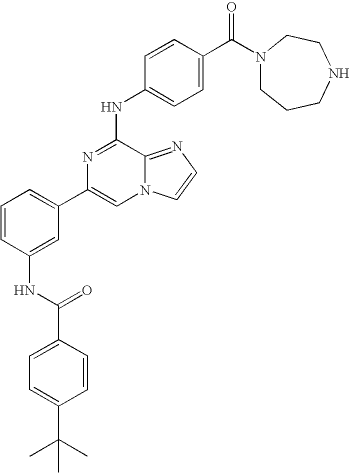 | 4-tert-Butyl-N-(3-{8-[4- ([1,4]diazepane-1-carbonyl)- phenylamino]-imidazo[1,2- a]pyrazin-6-yl}-phenyl)- benzamide C35H37N7O2 | 587.3 | 588.27 |
| 95 | 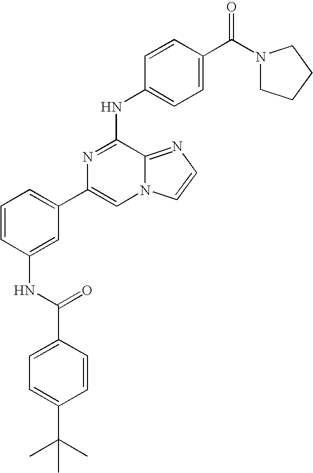 | 4-tert-Butyl-N-(3-{8-[4- (pyrrolidine-1-carbonyl)- phenylamino]-imidazo[1,2- a]pyrazin-6-yl}-phenyl)- benzamide C34H34N6O2 | 558.27 | 559.25 |
| 96 | 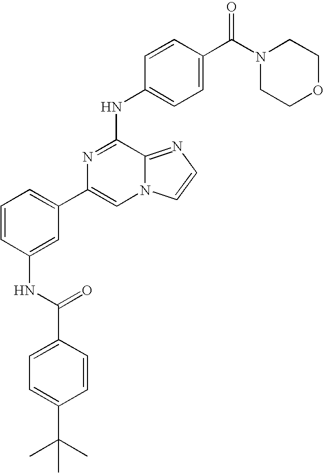 | 4-tert-Butyl-N-(3-{8-[4- (morpholine-4-carbonyl)- phenylamino]-imidazo[1,2- a]pyrazin-6-yl}-phenyl)- benzamide C34H34N6O3 | 574.26 | 575.24 |
| 97 | 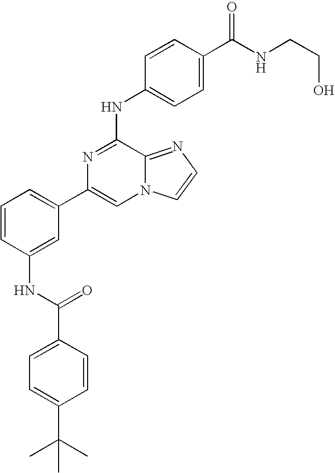 | 4-tert-Butyl-N-(3-{8-[4- (ethoxyamine-4-carbonyl)- phenylamino]-imidazo[1,2- a]pyrazin-6-yl}-phenyl)- benzamide C33H34N6O3 | 548.25 | 549.22 |
| 98 | 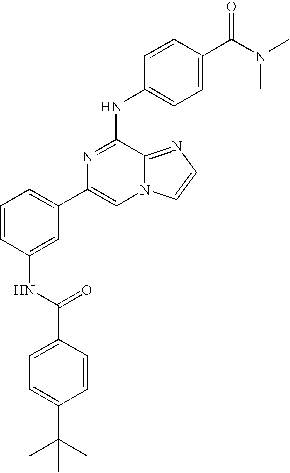 | 4-tert-Butyl-N-(3-{8-[4- (dimethylamine-4-carbonyl)- phenylamino]-imidazo[1,2- a]pyrazin-6-yl}-phenyl)- benzamide C32H32N6O2 | 532.25 | 533.22 |
| 99 | 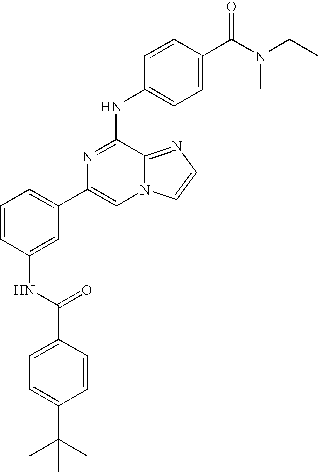 | 4-tert-Butyl-N-(3-{8-[4- (methylethylamine-4- carbonyl)-phenylamino]- imidazo[1,2-a]pyrazin-6-yl}- phenyl)-benzamide C33H34N6O2 | 546.27 | 547.24 |
| 100 | 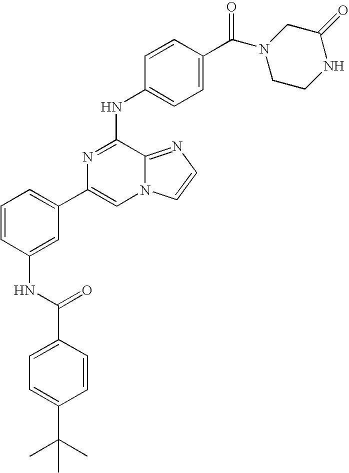 | 4-tert-Butyl-N-(3-{8-[4-(3- oxo-piperazine-1-carbonyl)- phenylamino]-imidazo[1,2- a]pyrazin-6-yl}-phenyl)- benzamide C34H33N7O3 | 587.26 | 588.28 |
| 101 | 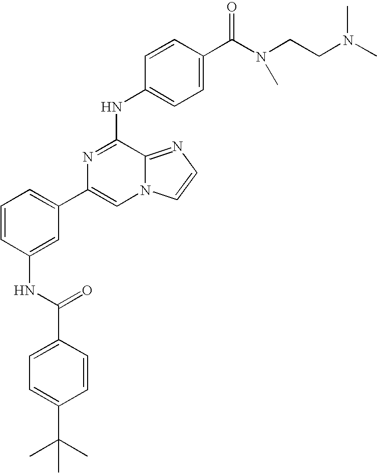 | 4-tert-Butyl-N-(3-{8- [N,N,N′-Trimethyl-ethane- 1,2-diamine-4-carbonyl)- phenylamino]-imidazo[1,2- a]pyrazin-6-yl}-phenyl)- benzamide C35H39N7O2 | 589.31 | 590.35 |
| 102 | 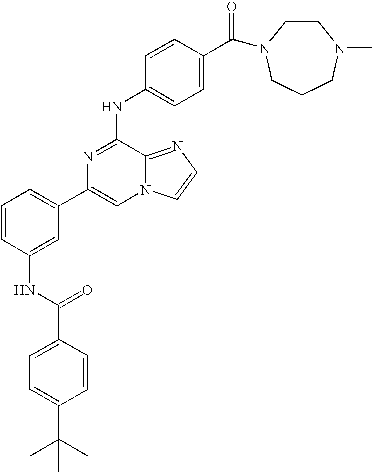 | 4-tert-Butyl-N-(3-{8-[4-(4- methyl-[1,4]diazepane-1- carbonyl)-phenylamino]- imidazo[1,2-a]pyrazin-6-yl}- phenyl)-benzamide C36H39N7O2 | 601.31 | 602.32 |

| | | | | MS m/z |
|---|---|---|---|---|
| Cmp. # | STRUCTURE | NAME | MW | (M⁺¹) |
| 103 | 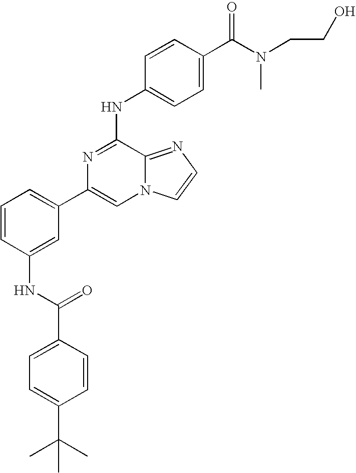 | 4-tert-Butyl-N-(3-{8-[4-(N- methyl ethoxyamine-4- carbonyl)-phenylamino]- imidazo[1,2-a]pyrazin-6-yl}- phenyl)-benzamide C33H34N6O3 | 562.26 | 563.29 |
| 104 | 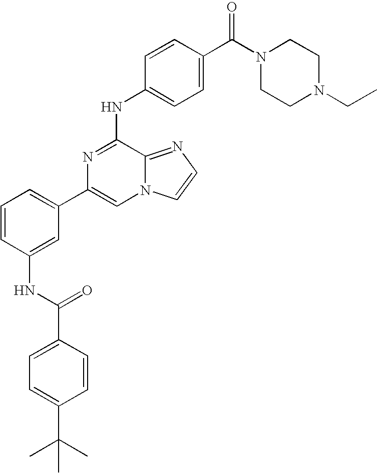 | 4-tert-Butyl-N-(3-{8-[4-(4- ethyl-piperazine-1- carbonyl)-phenylamino]- imidazo[1,2-a]pyrazin-6-yl}- phenyl)-benzamide C36H39N7O2 | 601.31 | 602.38 |
| 105 | 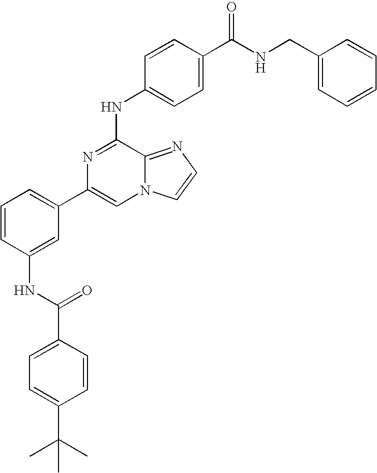 | 4-{6-[3-(4-tert-Butyl- benzoylamino)-phenyl]- imidazo[1,2-a]pyrazin-8- ylamino}-benzoic benzyl amide C37H34N6O2 | 595.26 | 596.32 |
| 106 | 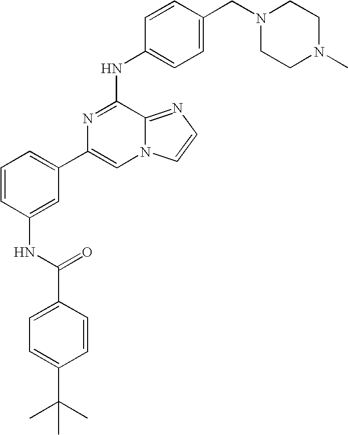 | 4-tert-Butyl-N-(3-{8-[4-(4- methyl-piperazin-1- ylmethyl)-phenylamino]- imidazo[1,2-a]pyrazin-6-yl}- phenyl)-benzamide C35H39N7O | 573.32 | 574.39 |
| 107 | 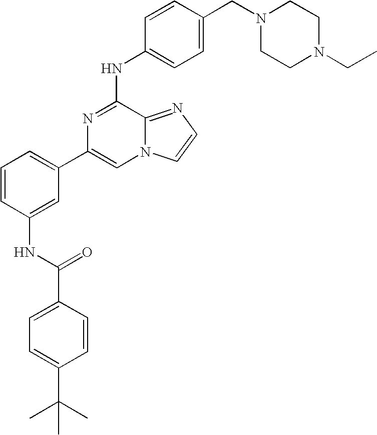 | 4-tert-Butyl-N-(3-{8-[4-(4- ethyl-piperazin-1-ylmethyl)- phenylamino]-imidazo[1,2- a]pyrazin-6-yl}-phenyl)- benzamide C36H41N7O | 587.33 | 588.42 |
| 108 | 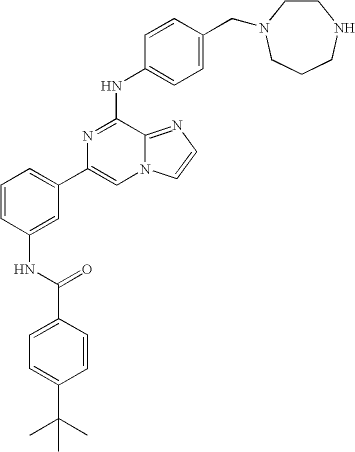 | 4-tert-Butyl-N-{3-[8-(4- [1,4]diazepan-1-ylmethyl- phenylamino)-imidazo[1,2- a]pyrazin-6-yl]-phenyl}- benzamide C35H39N7O | 573.32 | 574.39 |
| 109 | 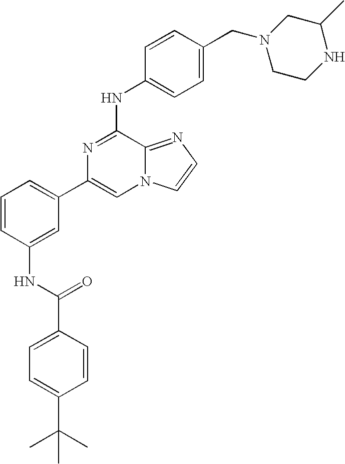 | 4-tert-Butyl-N-(3-{8-[4-(3- methyl-piperazin-1- ylmethyl)-phenylamino]- imidazo[1,2-a]pyrazin-6-yl}- phenyl)-benzamide C35H39N7O | 573.32 | 574.38 |
| 110 | 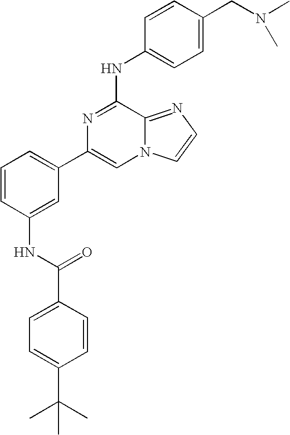 | 4-tert-Butyl-N-{3-[8-(4- dimethylaminomethyl- phenylamino)-imidazo[1,2- a]pyrazin-6-yl]-phenyl}- benzamide C32H34N6O | 518.27 | 519.33 |
| 111 | 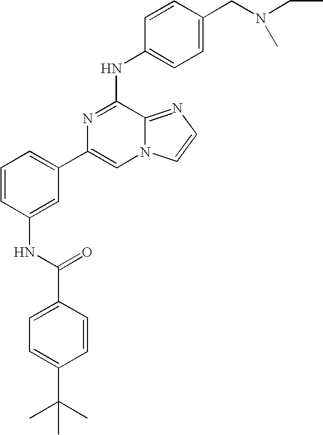 | 4-tert-Butyl-N-[3-(8-{4- [(ethyl-methyl-amino)- methyl]-phenylamino}- imidazo[1,2-a]pyrazin-6-yl)- phenyl]-benzamide C33H36N6O | 532.29 | 533.33 |
| 112 | 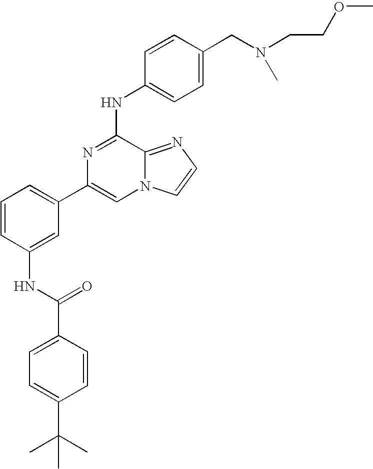 | 4-tert-Butyl-N-{3-[8-(4- {[(2-methoxy-ethyl)-methyl- amino]-methyl}- phenylamino)-imidazo[1,2- a]pyrazin-6-yl]-phenyl}- benzamide C34H38N6O2 | 562.31 | 563.33 |
| 113 | 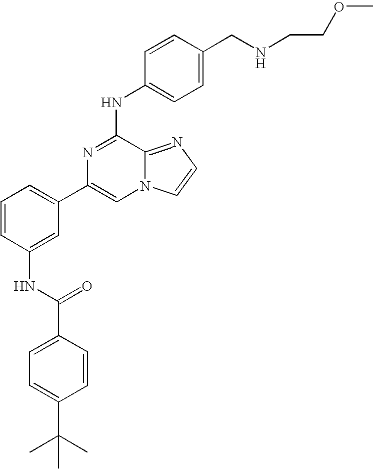 | 4-tert-Butyl-N-[3-(8-{4-[(2- methoxy-ethylamino)- methyl]-phenylamino}- imidazo[1,2-a]pyrazin-6-yl)- phenyl]-benzamide C33H36N6O2 | 548.28 | 549.32 |
| 114 | 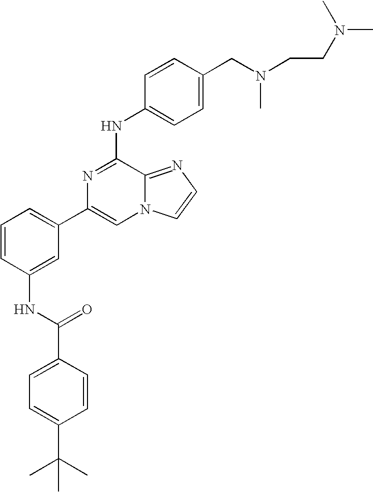 | 4-tert-Butyl-N-{3-[8-(4- {[(2-dimethylamino-ethyl)- methyl-amino]-methyl}- phenylamino)-imidazo[1,2- a]pyrazin-6-yl]-phenyl}- benzamide C35H41N7O | 575.33 | 576.39 |
| 115 | 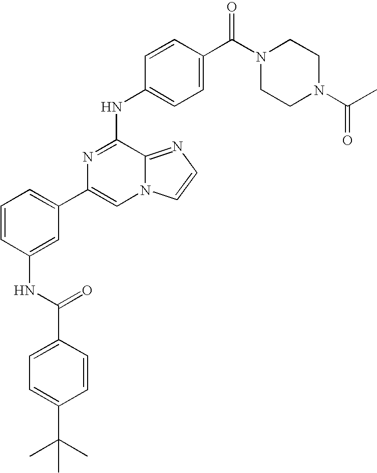 | N-(3-{8-[4-(4-Acetyl- piperazine-1-carbonyl)- phenylamino]-imidazo[1,2- a]pyrazin-6-yl}-phenyl)-4- tert-butyl-benzamide C36H37N7O3 | 615.29 | 616.31 |
| 116 | 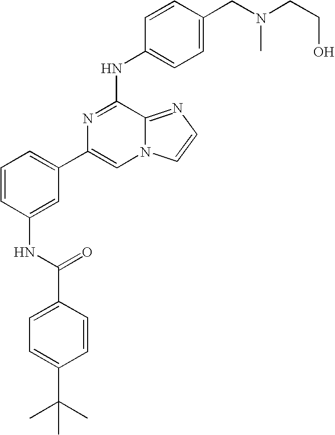 | 4-tert-Butyl-N-{3-[8-(4- {[(2-hydroxy-ethyl)-methyl- amino]-methyl}- phenylamino)-imidazo[1,2- a]pyrazin-6-yl]-phenyl}- benzamide C33H36N6O2 | 548.28 | 549.32 |
| 117 | 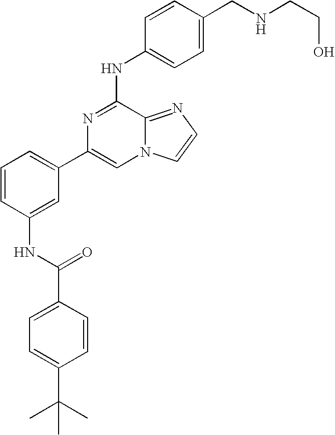 | 4-tert-Butyl-N-[3-(8-{4-[(2- hydroxy-ethylamino)- methyl]-phenylamino}- imidazo[1,2-a]pyrazin-6-yl)- phenyl]-benzamide C32H34N6O2 | 534.27 | 535.32 |
| 118 | 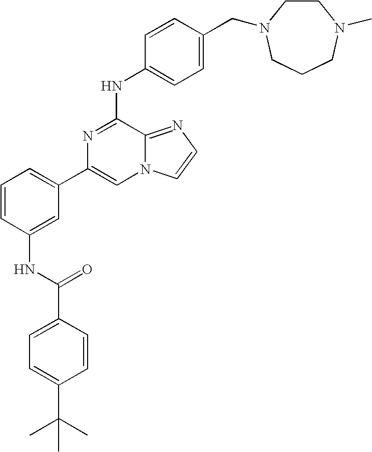 | 4-tert-Butyl-N-(3-{8-[4-(4- methyl-[1,4]diazepan-1- ylmethyl)-phenylamino]- imidazo[1,2-a]pyrazin-6-yl}- phenyl)-benzamide C36H41N7O | 587.33 | 588.37 |
| 119 | 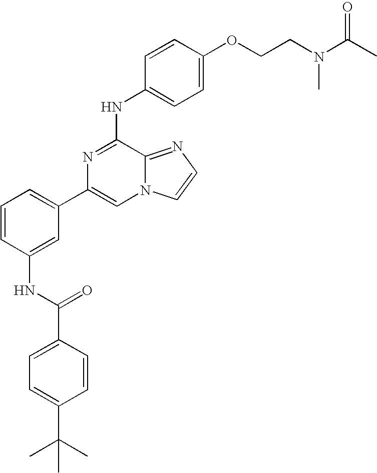 | N-[3-(8-{4-[2-(Acetyl- methyl-amino)-ethoxy]- phenylamino}-imidazo[1,2- a]pyrazin-6-yl)-phenyl]-4- tert-butyl-benzamide C34H36N6O3 | 576.28 | 577.32 |
| 120 | 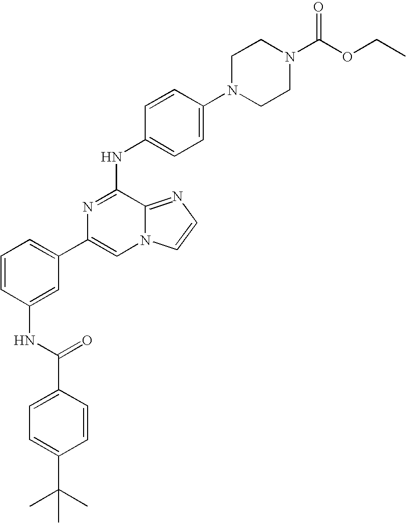 | 4-(4-{6-[3-(4-tert-Butyl- benzoylamino)-phenyl]- imidazo[1,2-a]pyrazin-8- ylamino}-phenyl)- piperazine-1-carboxylic acid ethyl ester C36H39N7O3 | 617.31 | 618.31 |
| 121 | 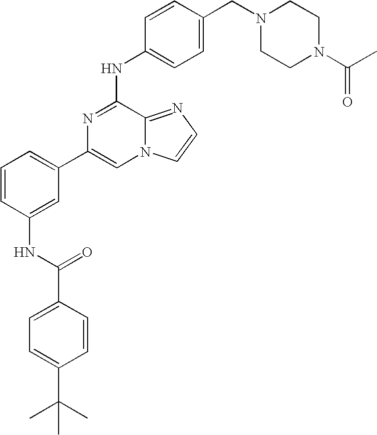 | N-(3-{8-[4-(4-Acetyl- piperazin-1-ylmethyl)- phenylamino]-imidazo[1,2- a]pyrazin-6-yl}-phenyl)-4- tert-butyl-benzamide C36H39N7O2 | 601.31 | 602.26 |
| 122 | 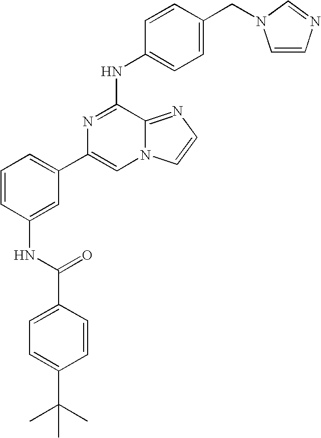 | 4-tert-Butyl-N-{3-[8-(4- imidazol-1-ylmethyl- phenylamino)-imidazo[1,2- a]pyrazin-6-yl]-phenyl}- benzamide C33H31N7O | 541.25 | 542.27 |
| 123 | 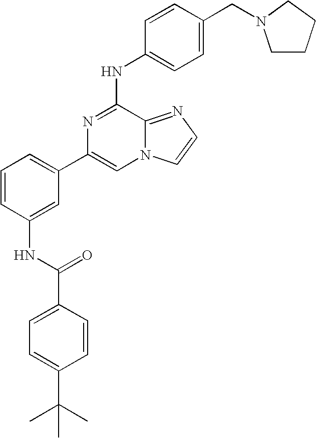 | 4-tert-Butyl-N-{3-[8-(4- pyrrolidin-1-ylmethyl- phenylamino)-imidazo[1,2- a]pyrazin-6-yl]-phenyl}- benzamide C34H36N6O | 544.29 | 545.3 |
| 124 | 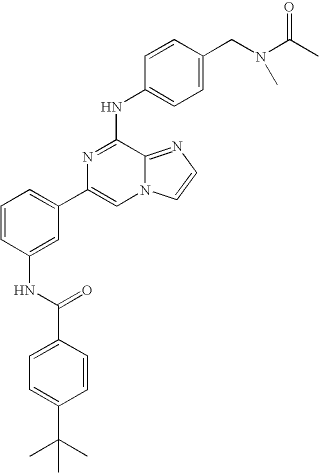 | N-[3-(8-{4-[(Acetyl-methyl- amino)-methyl]- phenylamino}-imidazo[1,2- a]pyrazin-6-yl)-phenyl]-4- tert-butyl-benzamide C33H34N6O2 | 546.27 | 547.33 |
| 125 | 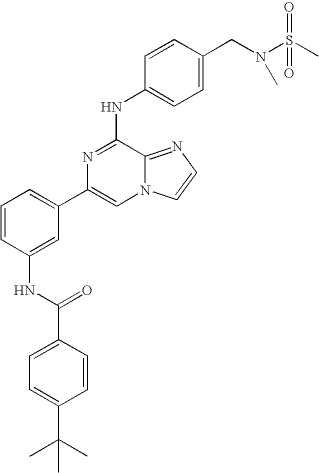 | 4-tert-Butyl-N-[3-(8-{4- [(methanesulfonyl-methyl- amino)-methyl]- phenylamino}-imidazo[1,2- a]pyrazin-6-yl)-phenyl]- benzamide C32H34N6O3S | 582.24 | 583.27 |
| 126 | 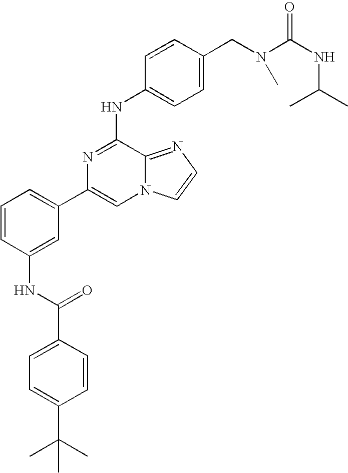 | 4-tert-Butyl-N-(3-{8-[4-(3- isopropyl-1-methyl- ureidomethyl)- phenylamino]-imidazo[1,2- a]pyrazin-6-yl}-phenyl)- benzamide C35H39N7O2 | 589.31 | 590.35 |
| 127 | 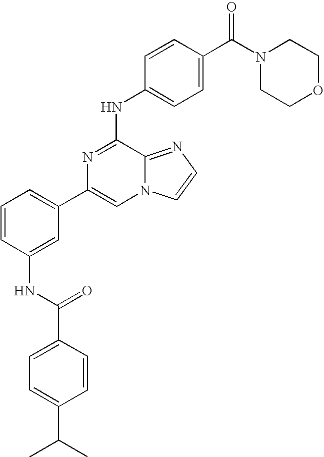 | 4-Isopropyl-N-(3-{8-[4- (morpholine-4-carbonyl)- phenylamino]-imidazo[1,2- a]pyrazin-6-yl}-phenyl)- benzamide C33H32N6O3 | 560.25 | 561.28 |
| 128 | 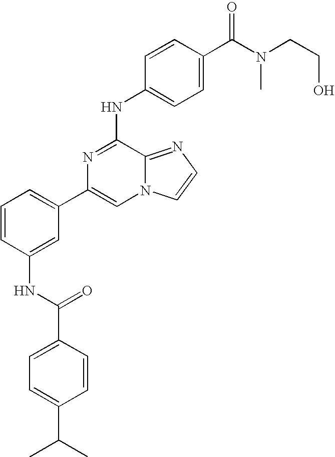 | N-{3-[8-(4-{[(2-Hydroxy- ethyl)-methyl-amino]- carbonyl}-phenylamino)- imidazo[1,2-a]pyrazin-6-yl]- phenyl}-4-isopropyl- benzamide C32H32N6O3 | 548.25 | 549.25 |
| 129 | 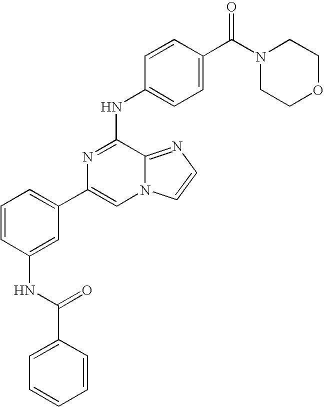 | N-(3-{8-[4-(Morpholine-4- carbonyl)-phenylamino]- imidazo[1,2-a]pyrazin-6-yl}- phenyl)-benzamide C30H26N6O3 | 518.2 | 519.22 |
| 130 | 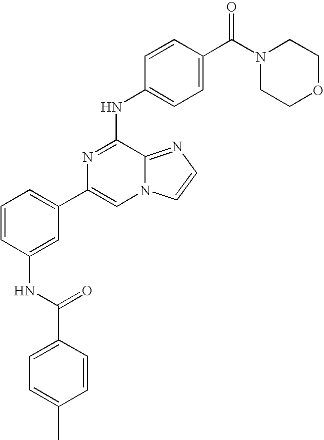 | 4-Methyl-N-(3-{8-[4- (morpholine-4-carbonyl)- phenylamino]-imidazo[1,2- a]pyrazin-6-yl}-phenyl)- benzamide C31H28N6O3 | 532.22 | 533.17 |
| 131 | 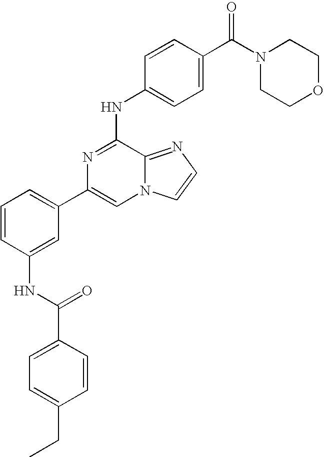 | 4-Ethyl-N-(3-{8-[4- (morpholine-4-carbonyl)- phenylamino]-imidazo[1,2- a]pyrazin-6-yl}-phenyl)- benzamide C32H30N6O3 | 546.23 | 547.17 |
| 132 | 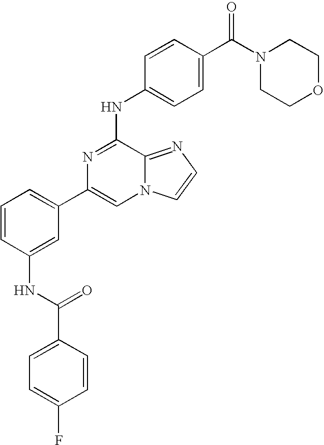 | 4-Fluoro-N-(3-{8-[4- (morpholine-4-carbonyl)- phenylamino]-imidazo[1,2- a]pyrazin-6-yl}-phenyl)- benzamide C30H25FN6O3 | 536.19 | 537.21 |
| 133 | 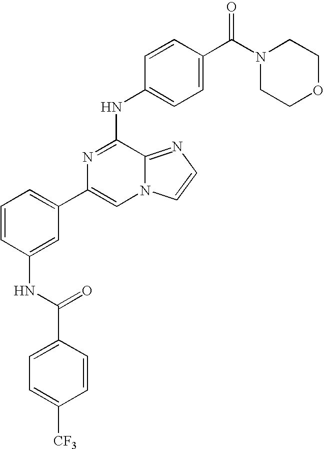 | N-(3-{8-[4-(Morpholine-4- carbonyl)-phenylamino]- imidazo[1,2-a]pyrazin-6-yl}- phenyl)-4-trifluoromethyl- benzamide C31H25F3N6O3 | 586.19 | 587.2 |
| 134 | 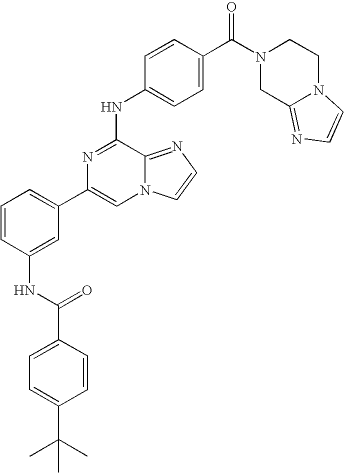 | 4-tert-Butyl-N-(3-{8-[4-(5,6- dihydro-8H-imidazo[1,2- a]pyrazine-7-carbonyl)- phenylamino]-imidazo[1,2- a]pyrazin-6-yl}-phenyl)- benzamide C36H34N8O2 | 610.28 | 611.21 |
| 135 | 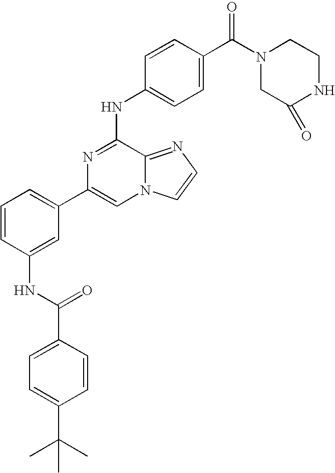 | 4-tert-Butyl-N-(3-{8-[4-(3- oxo-piperazin-1-ylmethyl)- phenylamino]-imidazo[1,2- a]pyrazin-6-yl}-phenyl)- benzamide C34H35N7O2 | 573.28 | 574.18 |
| 136 | 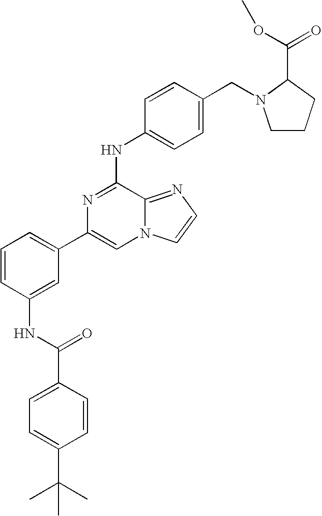 | 1-(4-{6-[3-(4-tert-Butyl- benzoylamino)-phenyl]- imidazo[1,2-a]pyrazin-8- ylamino}-benzyl)- pyrrolidine-2-carboxylic acid methyl ester C36H38N6O3 | 602.3 | 603.39 |
| 137 | 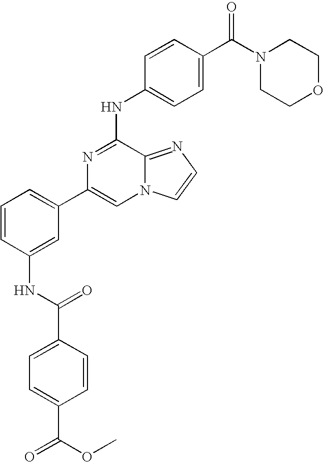 | N-(3-{8-[4-(Morpholine-4- carbonyl)-phenylamino]- imidazo[1,2-a]pyrazin-6-yl}- phenyl)-terephthalamic acid methyl ester C32H28N6O5 | 576.21 | 577.36 |
| 138 | 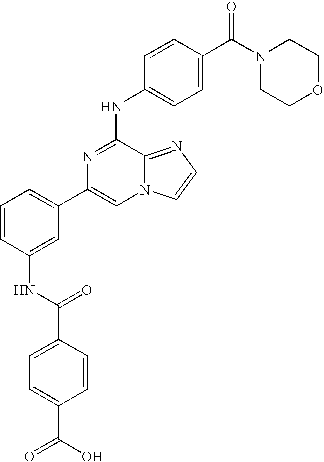 | N-(3-{8-[4-(Morpholine-4- carbonyl)-phenylamino]- imidazo[1,2-a]pyrazin-6-yl}- phenyl)-terephthalamic acid C31H26N6O5 | 562.19 | 563.33 |
| 139 | 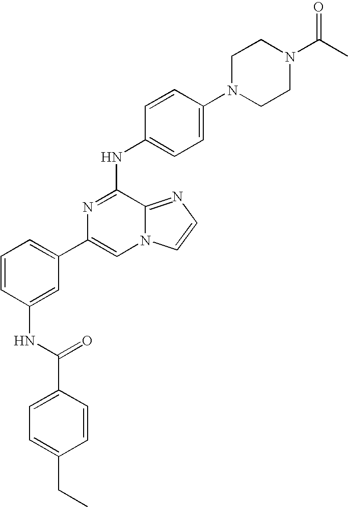 | N-(3-{8[4-(4-Acetyl- piperazin-1-yl)- phenylamino]-imidazo[1,2- a]pyrazin-6-yl}-phenyl)-4- ethyl-benzamide C33H33N7O2 | 559.26 | 560.25 |
| 140 | 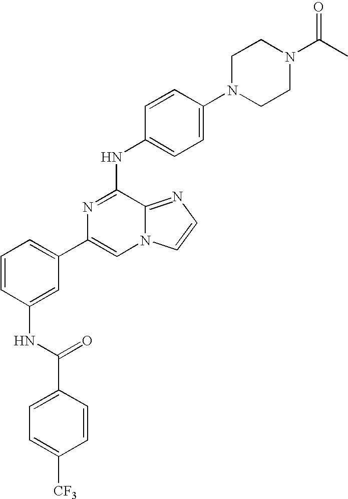 | N-(3-{8-[4-(4-Acetyl- piperazin-1-yl)- phenylamino]-imidazo[1,2- a]pyrazin-6-yl}-phenyl)-4- trifluoromethyl-benzamide C32H28F3N7O2 | 599.22 | 600.22 |
| 141 | 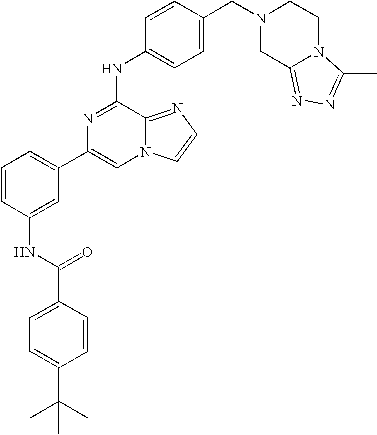 | 4-tert-Butyl-N-(3-{8-[4-(3- methyl-5,6-dihydro-8H- [1,2,4]triazolo[4,3-a]pyrazin- 7-ylmethyl)-phenylamino]- imidazo[1,2-a]pyrazin-6-yl}- phenyl)-benzamide C36H37N9O | 611.31 | 612.41 |
| 142 | 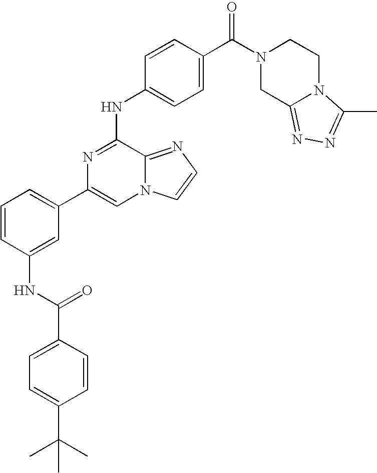 | 4-tert-Butyl-N-(3-{8-[4-(3- methyl-5,6-dihydro-8H- [1,2,4]triazolo[4,3- a]pyrazine-7-carbonyl)- phenylamino]-imidazo[1,2- a]pyrazin-6-yl}-phenyl)- benzamide C36H35N9O2 | 625.29 | 626.4 |
| 143 | 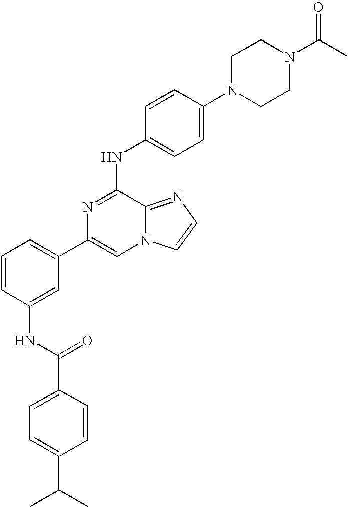 | N-(3-{8-[4-(4-Acetyl- piperazin-1-yl)- phenylamino]-imidazo[1,2- a]pyrazin-6-yl}-phenyl)-4- isopropyl-benzamide C34H35N7O2 | 573.28 | 574.38 |
| 144 | 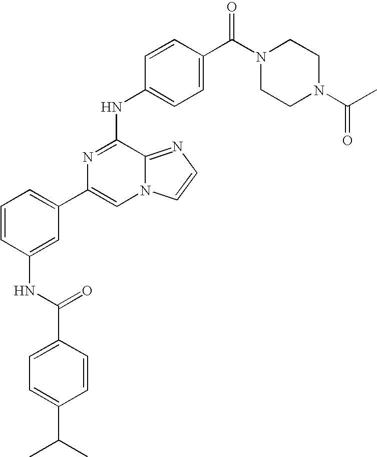 | N-(3-{8-[4-(4-Acetyl- piperazine-1-carbonyl)- phenylamino]-imidazo[1,2- a]pyrazin-6-yl}-phenyl)-4- isopropyl-benzamide C35H35N7O3 | 601.28 | 602.27 |
| 145 | 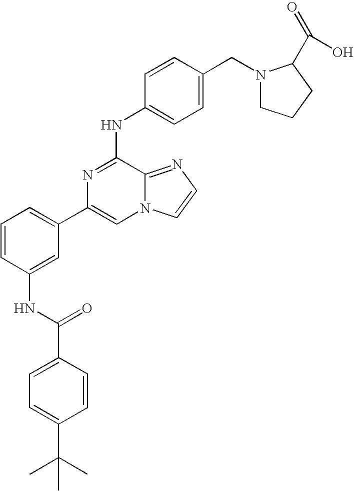 | 1-(4-{6-[3-(4-tert-Butyl- benzoylamino)-phenyl]- imidazo[1,2-a]pyrazin-8- ylamino)-benzyl)- pyrrolidine-2-carboxylic acid C35H36N6O3 | 588.28 | 589.25 |
| 146 | 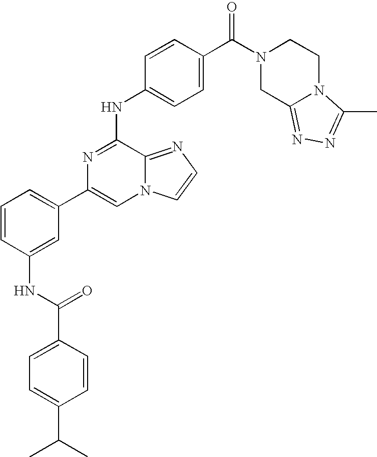 | 4-Isopropyl-N-(3-{8-[4-(3- methyl-5,6-dihydro-8H- [1,2,4]triazolo[4,3- a]pyrazine-7-carbonyl)- phenylamino]-imidazo[1,2- a]pyrazin-6-yl}-phenyl)- benzamide C35H33N9O2 | 611.27 | 612.26 |
| 147 | 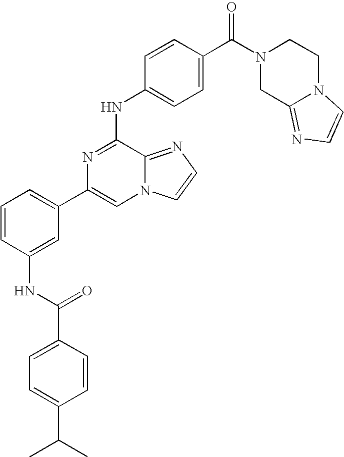 | N-(3-{8-[4-(5,6-Dihydro- 8H-imidazo[1,2-a]pyrazine- 7-carbonyl)-phenylamino]- imidazo[1,2-a]pyrazin-6-yl}- phenyl)-4-isopropyl- benzamide C35H32N8O2 | 596.26 | 597.26 |
| 148 | 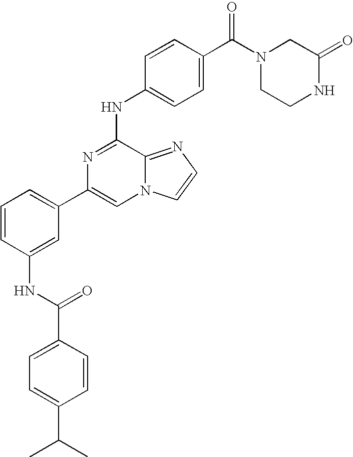 | 4-Isopropyl-N-(3-{8-[4-(3- oxo-piperazine-1-carbonyl)- phenylamino]-imidazo[1,2- a]pyrazin-6-yl}-phenyl)- benzamide C33H31N7O3 | 573.24 | 574.27 |
| 149 | 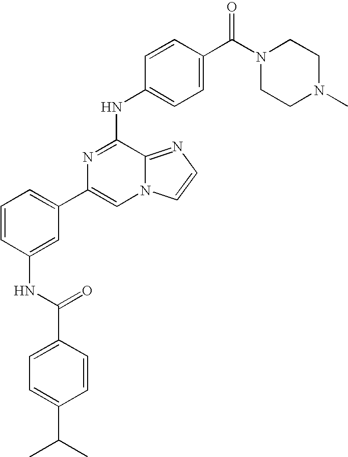 | 4-Isopropyl-N-(3-{8-[4-(4- methyl-piperazine-1- carbonyl)-phenylamino]- imidazo[1,2-a]pyrazin-6-yl}- phenyl)-benzamide C34H35N7O2 | 573.28 | 574.29 |
| 150 | 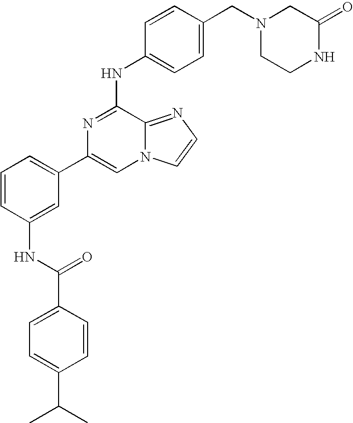 | 4-Isopropyl-N-(3-{8-[4-(3- oxo-piperazin-1-ylmethyl)- phenylamino]-imidazo[1,2- a]pyrazin-6-yl}-phenyl)- benzamide C33H33N7O2 | 559.26 | 560.29 |
| 151 | 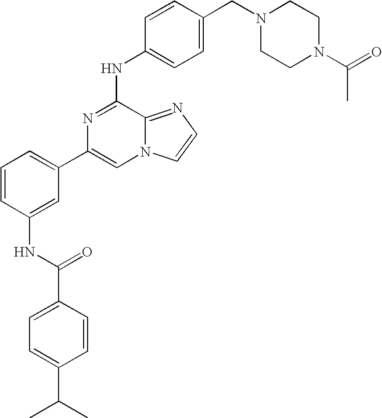 | N-(3-{8-[4-(4-Acetyl- piperazin-1-ylmethyl)- phenylamino]-imidazo[1,2- a]pyrazin-6-yl}-phenyl)-4- isopropyl-benzamide C35H37N7O2 | 587.3 | 588.31 |
| 152 | 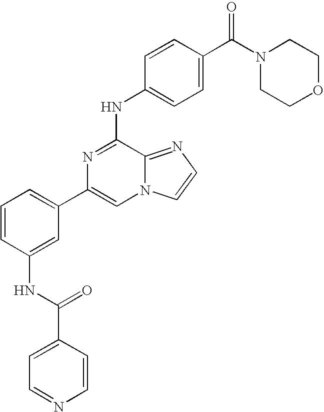 | N-(3-{8-[4-(Morpholine-4- carbonyl)-phenylamino]- imidazo[1,2-a]pyrazin-6-yl}- phenyl)-isonicotinamide C29H25N7O3 | 519.2 | 520.2 |
| 153 | 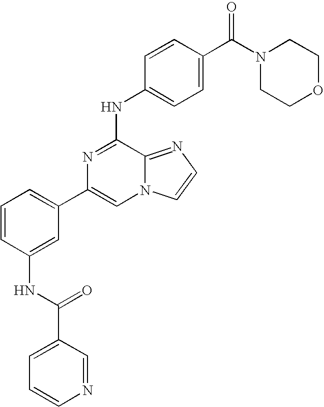 | N-(3-{8-[4-(Morpholine-4- carbonyl)-phenylamino]- imidazo[1,2-a]pyrazin-6-yl}- phenyl)-nicotinamide C29H25N7O3 | 519.2 | 520.19 |
| 154 | 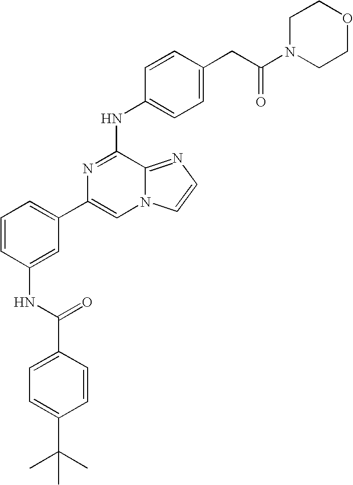 | 4-tert-Butyl-N-(3-{8-[4-(2- morpholin-4-yl-2-oxo- ethyl)-phenylamino]- imidazo[1,2-a]pyrazin-6-yl}- phenyl)-benzamide C35H36N6O3 | 588.28 | 589.28 |
| 155 | 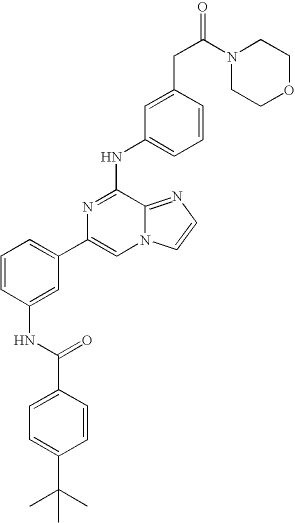 | 4-tert-Butyl-N-(3-{8-[3-(2- morpholin-4-yl-2-oxo- ethyl)-phenylamino]- imidazo[1,2-a]pyrazin-6-yl}- phenyl)-benzamide C35H36N6O3 | 588.28 | 589.29 |
| 156 | 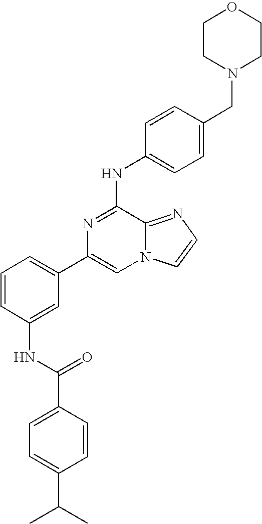 | 4-Isopropyl-N-{3-[8-(4- morpholin-4-ylmethyl- phenylamino)-imidazo[1,2- a]pyrazin-6-yl]-phenyl}- benzamide C33H34N6O2 | 546.27 | 547.28 |
| 157 | 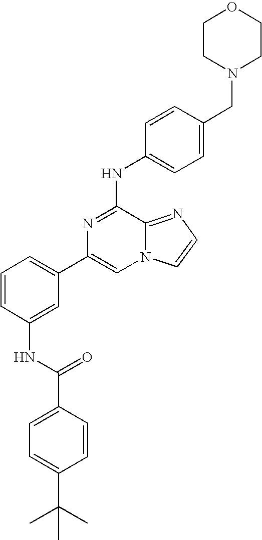 | 4-tert-Butyl-N-{3-[8-(4- morpholin-4-ylmethyl- phenylamino)-imidazo[1,2- a]pyrazin-6-yl]-phenyl}- benzamide C34H36N6O2 | 560.28 | 561.43 |
| 158 | 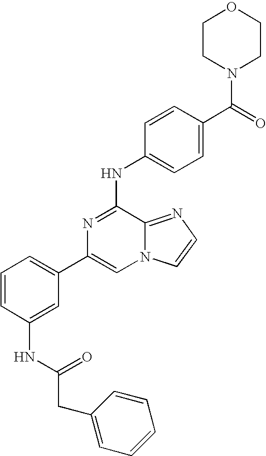 | N-(3-{8-[4-(Morpholine-4- carbonyl)-phenylamino]- imidazo[1,2-a]pyrazin-6-yl}- phenyl)-2-phenyl-acetamide C31H28N6O3 | 532.22 | 533.21 |
| 159 | 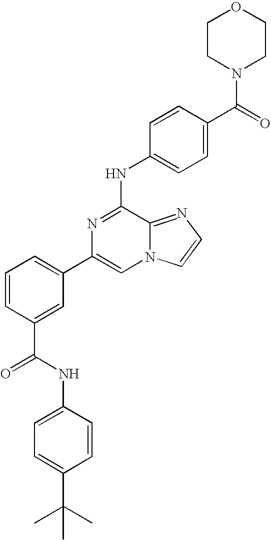 | N-(4-tert-Butyl-phenyl)-3- {8-[4-(morpholine-4- carbonyl)-phenylamino]- imidazo[1,2-a]pyrazin-6-yl}- benzamide C34H34N6O3 | 574.27 | 575.23 |
| 160 | 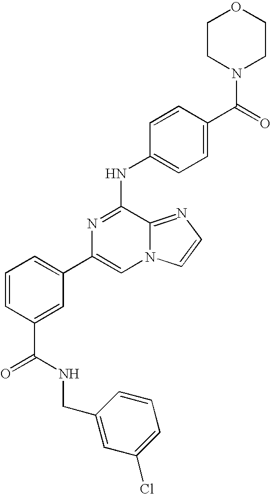 | N-(3-Chloro-benzyl)-3-{8- [4-(morpholine-4-carbonyl)- phenylamino]-imidazo[1,2- a]pyrazin-6-yl}-benzamide C31H27ClN6O3 | 566.18 | 567.17 |
| 161 | 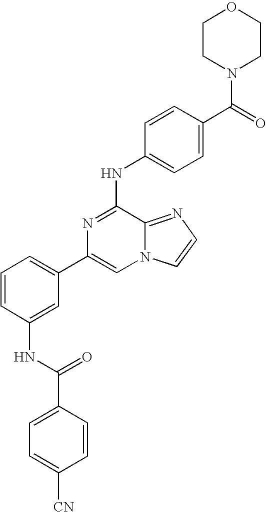 | 4-Cyano-N-(3-{8-[4- (morpholine-4-carbonyl)- phenylamino]-imidazo[1,2- a]pyrazin-6-yl}-phenyl)- benzamide C31H25N7O3 | 543.2 | 544.32 |
| 162 | 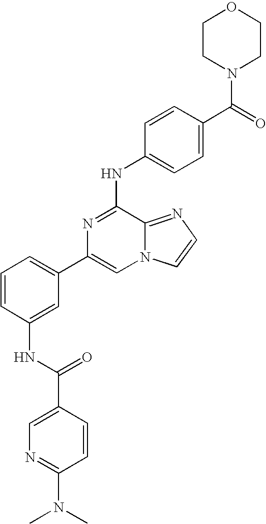 | 6-Dimethylamino-N-(3-{8- [4-(morpholine-4-carbonyl)- phenylamino]-imidazo[1,2- a]pyrazin-6-yl}-phenyl)- nicotinamide C31H30N8O3 | 562.24 | 563.26 |
| 163 | 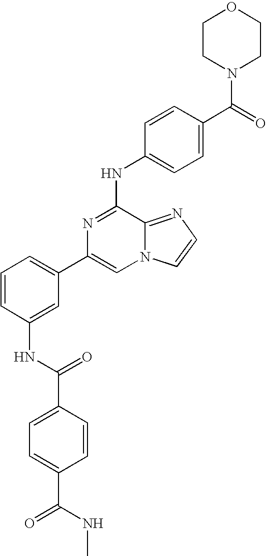 | N-Methyl-N′-(3-{8-[4- (morpholine-4-carbonyl)- phenylamino]-imidazo[1,2- a]pyrazin-6-yl}-phenyl)- terephthalamide C32H29N7O4 | 575.22 | 576.25 |
| 164 | 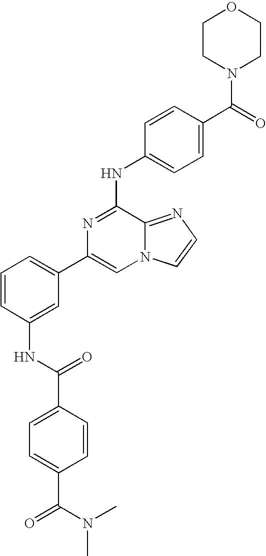 | N,N-Dimethyl-N′-(3-{8-[4- (morpholine-4-carbonyl)- phenylamino]-imidazo[1,2- a]pyrazin-6-yl}-phenyl)- terephthalamide C33H31N7O4 | 589.24 | 590.26 |
| 165 | 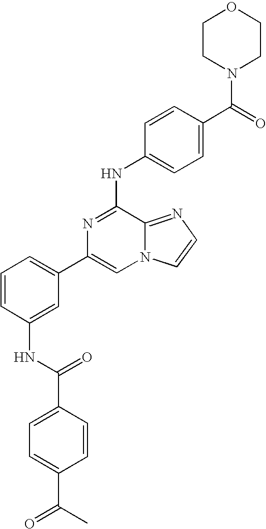 | 4-Acetyl-N-(3-{8-[4- (morpholine-4-carbonyl)- phenylamino]-imidazo[1,2- a]pyrazin-6-yl}-phenyl)- benzamide C32H28N6O4 | 560.21 | 561.25 |
| 166 | 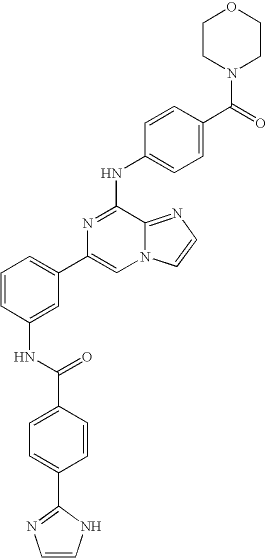 | 4-(1H-Imidazol-2-yl)-N-(3- {8-[4-(morpholine-4- carbonyl)-phenylamino]- imidazo[1,2-a]pyrazin-6-yl}- phenyl)-benzamide C33H28N8O3 | 584.22 | 585.31 |
| 167 | 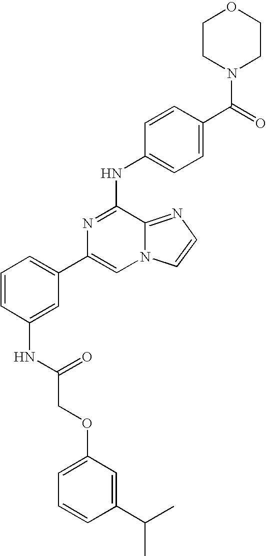 | 2-(3-Isopropyl-phenoxy)-N- (3-{8-[4-(morpholine-4- carbonyl)-phenylamino]- imidazo[1,2-a]pyrazin-6-yl}- phenyl)-acetamide C34H34N6O4 | 590.26 | 591.32 |
| 168 | 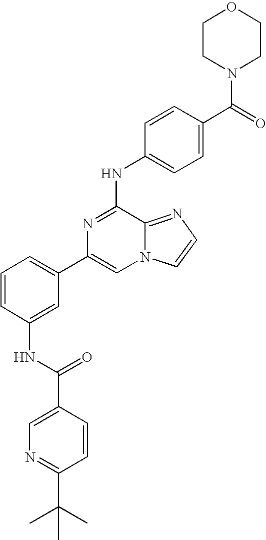 | 6-tert-Butyl-N-(3-{8-[4- (morpholine-4-carbonyl)- phenylamino]-imidazo[1,2- a]pyrazin-6-yl}-phenyl)- nicotinamide C33H33N7O3 | 575.26 | 576.24 |
| 169 | 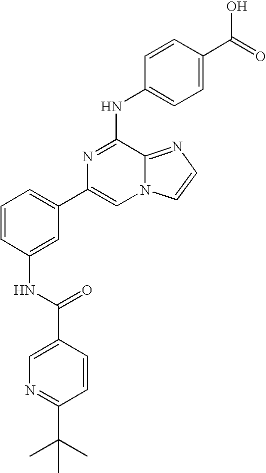 | 4-(6-{3-[(6-tert-Butyl- pyridine-3-carbonyl)- amino]-phenyl}- imidazo[1,2a]pyrazin-8-ylamino)-benzoic acid C29H26N6O3 | 506.2 | 507.21 |
| 170 | 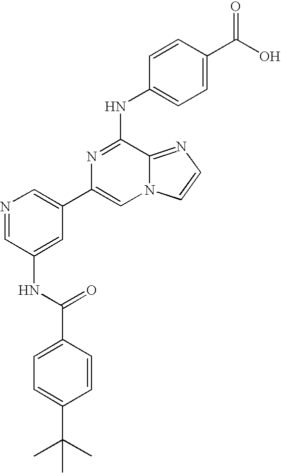 | 4-{6-[5-(4-tert-Butyl- benzoylamino)-pyridin-3- yl]-imidazo[1,2-a]pyrazin-8- ylamino}-benzoic acid C29H26N6O3 | 506.21 | 507.07 |
| 171 | 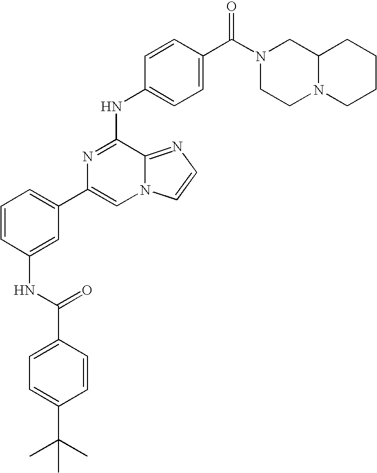 | 4-tert-Butyl-N-(3-{8-[4- (octahydro-pyrido[1,2- a]pyrazine-2-carbonyl)- phenylamino]-imidazo[1,2- a]pyrazin-6-yl}-phenyl)- benzamide C38H41N7O2 | 627.33 | 628.46 |
| 172 | 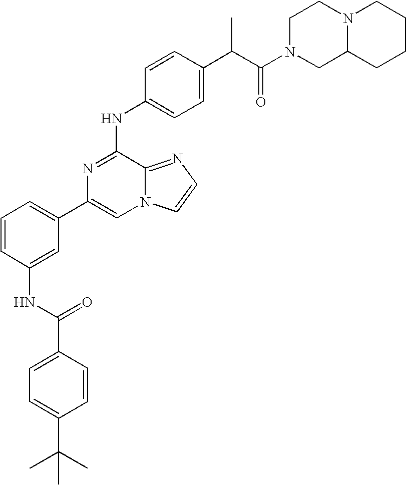 | 4-tert-Butyl-N-[3-(8-{4-[1- methyl-2-(octahydro- [1,2-a]pyrazin-2-yl)-2- oxo-ethyl]-phenylamino}- imidazo[1,2-a]pyrazin-6-yl)- phenyl]-benzamide C40H45N7O2 | 655.36 | 656.44 |
| 173 | 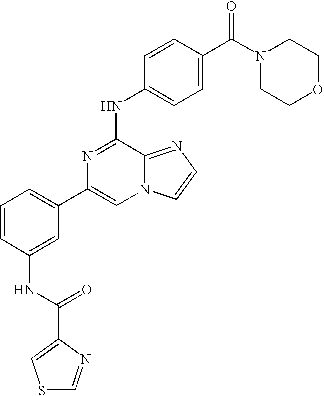 | Thiazole-4-carboxylic acid (3-{8-[4-(morpholine-4- carbonyl)-phenylamino]- imidazo[1,2-a]pyrazin-6- yl}-phenyl)-amide C27H23N7O3S | 525.16 | 526.21 |
| 174 | 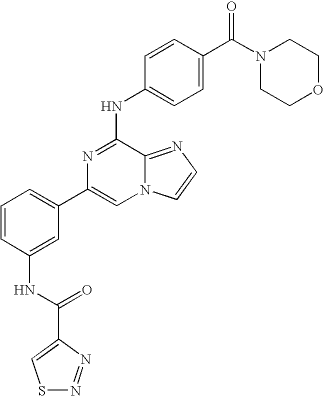 | [1,2,3]Thiadiazole-4- carboxylic acid (3-{8-[4- (morpholine-4-carbonyl)- phenylamino]-imidazo[1,2- a]pyrazin-6-yl}-phenyl)- amide C26H22N8O3S | 526.15 | 527.35 |
| 175 | 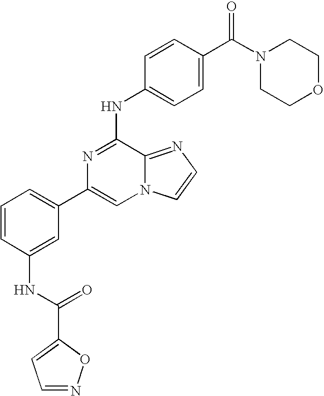 | Isoxazole-5-carboxylic acid (3-{8-[4-(morpholine-4- carbonyl)-phenylamino]- imidazo[1,2-a]pyrazin-6- yl)-phenyl)-amide C27H23N7O4 | 509.18 | 510.54 |
| 176 | 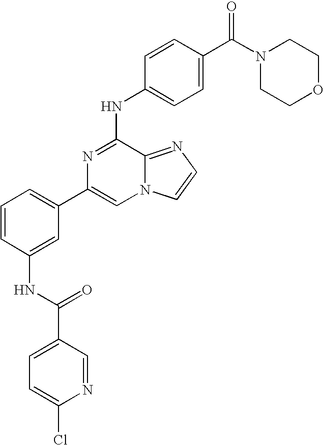 | 6-Chloro-N-(3-{8-[4- (morpholine-4-carbonyl)- phenylamino]-imidazo[1,2- a]pyrazin-6-yl}-phenyl)- nicotinamide C29H24ClN7O3 | 553.16 | 554.6 |
| 177 | 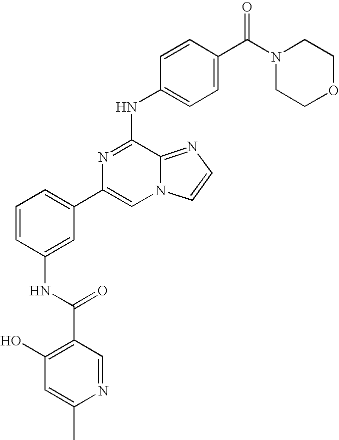 | 4-Hydroxy-6-methyl-N-(3- {8-[4-(morpholine-4- carbonyl)-phenylamino]- imidazo[1,2-a]pyrazin-6-yl}- phenyl)-nicotinamide C30H27N7O4 | 549.21 | 550.31 |

The compounds disclosed in synthetic Examples 1 to 4 are tested in the Btk biochemical assay described herein and exhibit an IC₅₀ value less than or equal to 1 micromolar. Certain of those compounds exhibit an IC₅₀ value less than or equal to 100 nM. Certain of those compounds exhibit an IC₅₀ value less than or equal to 10 nM.

Some of the compounds disclosed in synthetic Examples 1 to 4 are tested in the B-cell proliferation assay and exhibit an IC₅₀ value less than or equal to 10 micromolar. Certain of those compounds exhibit an IC₅₀ value less than or equal to 1 micromolar. Certain of those compounds exhibit an IC₅₀ value less than or equal to 500 nM in this assay. Certain of those compounds do not inhibit T-cell proliferation and have IC₅₀ values greater than or equal to 5 micromolar when assayed under conditions described herein. Certain compounds disclosed in Examples 1 to 4 exhibit IC₅₀ values for inhibition of T-cell proliferation that were at least 3-fold, and in some instances 5-fold, or even 10-fold greater than the IC₅₀ values of those compounds for inhibition of B-cell proliferation.

### Example 6

### Biochemical Btk Assay

A generalized procedure for one standard biochemical Btk Kinase Assay that can be used to test compounds disclosed in this application is as follows.

A master mix minus Btk enzyme is prepared containing 1× Cell Signaling kinase buffer (25 mM Tris-HCl, pH 7.5, 5 mM beta-glycerophosphate, 2 mM dithiothreitol, 0.1 mM Na₃VO₄, 10 mM MgCl₂), 0.5 μM Promega PTK Biotinylated peptide substrate 2, and 0.01% BSA. A master mix plus Btk enzyme is prepared containing 1× Cell Signaling kinase buffer, 0.5 μM PTK Biotinylated peptide substrate 2, 0.01% BSA, and 100 ng/well (0.06 mU/well) Btk enzyme. Btk enzyme is prepared as follows: full length human wildtype Btk (accession number NM-000061) with a C-terminal V5 and 6×His tag was subcloned into pFastBac vector for making baculovirus carrying this epitope-tagged Btk. Generation of baculovirus was done based on Invitrogen's instructions detailed in its published protocol “Bac-toBac Baculovirus Expression Systems” (Cat. Nos. 10359-016 and 10608-016). Passage 3 virus was used to infect Sf9 cells to overexpress the recombinant Btk protein. The Btk protein was then purified to homogeneity using Ni-NTA column. The purity of the final protein preparation was greater than 95% based on the sensitive Sypro-Ruby staining. A solution of 200 μM ATP is prepared in water and adjusted to pH7.4 with 1N NaOH. A quantity of 1.25 μL of compounds in 5% DMSO is transferred to a 96-well ½ area Costar polystyrene plate. Compounds are tested singly and with an 11-point dose-responsive curve (starting concentration is 10 μM; 1:2 dilution). A quantity of 18.75 μL of master mix minus enzyme (as a negative control) and master mix plus enzyme is transferred to appropriate wells in 96-well ½ area costar polystyrene plate. 5 μL of 200 μM ATP is added to that mixture in the 96-well ½ area Costar polystyrene plate for final ATP concentration of 40 μM. The reaction is allowed to incubate for 1 hour at room temperature. The reaction is stopped with Perkin Elmer 1× detection buffer containing 30 mM EDTA, 20 nM SA-APC, and 1 nM PT66 Ab. The plate is read using time-resolved fluorescence with a Perkin Elmer Envision using excitation filter 330 nm, emission filter 665 nm, and 2^{nd} emission filter 615 nm. IC₅₀ values are subsequently calculated.

### Example 7

### Ramos Cell Btk Assay

Another generalized procedure for a standard cellular Btk Kinase Assay that can be used to test compounds disclosed in this application is as follows.

Ramos cells are incubated at a density of 0.5×10⁷ cells/ml in the presence of test compound for 1 hr at 37° C. Cells are then stimulated by incubating with 10 μg/ml anti-human IgM F(ab)₂ for 5 minutes at 37° C. Cells are pelleted, lysed, and a protein assay is performed on the cleared lysate. Equal protein amounts of each sample are subject to SDS-PAGE and western blotting with either anti-phosphoBtk(Tyr223) antibody (Cell Signaling Technology #3531) to assess Btk autophosphorylation or an anti-Btk antibody (BD Transduction Labs #611116) to control for total amounts of Btk in each lysate.

### Example 8

### B-Cell Proliferation Assay

A generalized procedure for a standard cellular B-cell proliferation assay that can be used to test compounds disclosed in this application is as follows.

B-cells are purified from spleens of 8-16 week old Balb/c mice using a B-cell isolation kit (Miltenyi Biotech, Cat # 130-090-862). Testing compounds are diluted in 0.25% DMSO and incubated with 2.5×10⁵ purified mouse splenic B-cells for 30 min prior to addition of 10 μg/ml of an anti-mouse IgM antibody (Southern Biotechnology Associates Cat # 1022-01) in a final volume of 100 μl. Following 24 hr incubation, 1 μCi ³H-thymidine is added and plates are incubated an additional 36 hr prior to harvest using the manufacturer's protocol for SPA[³H] thymidine uptake assay system (Amersham Biosciences # RPNQ 0130). SPA-bead based fluorescence is counted in a microbeta counter (Wallace Triplex 1450, Perkin Elmer).

### Example 9

### T Cell Proliferation Assay

A generalized procedure for a standard T cell proliferation assay that can be used to test compounds disclosed in this application is as follows.

T cells are purified from spleens of 8-16 week old Balb/c mice using a Pan T cell isolation kit (Miltenyi Biotech, Cat # 130-090-861). Testing compounds are diluted in 0.25% DMSO and incubated with 2.5×10⁵ purified mouse splenic T cells in a final volume of 100 μl in flat clear bottom plates precoated for 90 min at 37° C. with 10 μg/ml each of anti-CD3 (BD # 553057) and anti-CD28 (BD # 553294) antibodies. Following 24 hr incubation, 1 μCi ³H-thymidine is added and plates incubated an additional 36 hr prior to harvest using the manufacturer's protocol for SPA[³H] thymidine uptake assay system (Amersham Biosciences # RPNQ 0130). SPA-bead based fluorescence was counted in a microbeta counter (Wallace Triplex 1450, Perkin Elmer).

1. At least one chemical entity chosen from compounds of Formula 1 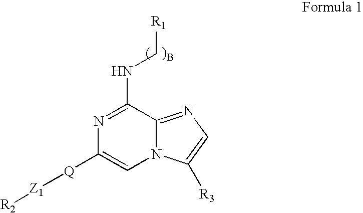
and pharmaceutically acceptable salts, solvates, crystal forms, chelates, non-covalent complexes, prodrugs, and mixtures thereof, wherein
B is chosen from 0 and 1;
R₁ is chosen from
substituted phenyl wherein the substituent is chosen from
hydroxy,
—CHO,
—COOH,
—CONH₂,
—CONHOH,
C₂-C₆ alkenyl,
substituted C₂-C₆ alkenyl chosen from mono-, di- and tri-substituted C₂-C₆ alkenyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di- and tri-substituted C₂-C₆ alkynyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di- and tri-substituted C₁-C₆alkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di- and tri-substituted C₁-C₆alkoxy, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di- and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di- and tri-substituted aryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di- and tri-substituted heteroaryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁,
substituted —C₁-C₆alkylNR₁₀(SO₂)R₁₁ chosen from mono-, di- and tri-substituted —C₁-C₆alkylNR₁₀(SO₂)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di- and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di- and tri-substituted —C₀-C₆alkyl(SO₂)R₁₀, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di- and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di- and tri-substituted C₁-C₂alkylene, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di- and tri-substituted C₀-C₂alkylene-O—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di- and tri-substituted —(C₁-C₂alkylene)(C═O)—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di- and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di- and tri-substituted C₃-C₇cycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di- and tri-substituted aryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, -mono-C₁-C₄alkylamino, di-C₁-C₄alkylamino, mono-C₁-C₄alkylcarboxamido, di-C₁-C₄alkylcarboxamido, and phenyl, and
heteroaryl, and
substituted heteroaryl chosen from mono-, di- and tri-substituted heteroaryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
substituted heteroaryl wherein the substituent is chosen from
hydroxy,
—CHO,
—COOH,
—CONH₂,
—CONHOH,
C₂-C₆ alkenyl,
substituted C₂-C₆ alkenyl chosen from mono-, di- and tri-substituted C₂-C₆ alkenyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di- and tri-substituted C₂-C₆ alkynyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di- and tri-substituted C₁-C₆alkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di- and tri-substituted C₁-C₆alkoxy, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di- and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di- and tri-substituted aryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di- and tri-substituted heteroaryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di- and tri-substituted —C₁-C₆alkylNR₁₀(SO₂)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di- and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di- and tri-substituted —C₀-C₆alkyl(SO₂)R₁₀, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di- and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di- and tri-substituted C₁-C₂alkylene, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di- and tri-substituted C₀-C₂alkylene-O—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di- and tri-substituted —(C₁-C₂alkylene)(C═O)—, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di- and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di- and tri-substituted C₃-C₇cycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di- and tri-substituted aryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
heteroaryl, and
substituted heteroaryl chosen from mono-, di- and tri-substituted heteroaryl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl, and
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl;
R₂ is chosen from
C₁-C₇ alkyl,
(C₁-C₆alkoxy)C₁-C₆alkoxy,
(heterocycloalkyl) C₀-C₂alkyl,
(C₃-C₇cycloalkyl)C₀-C₂alkyl,
(phenyl)C₀-C₂alkyl,
substituted (phenyl)C₀-C₂alkyl chosen from mono-, di-, and tri-substituted (phenyl)C₀-C₂alkyl, wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
(phenoxy)C₀-C₂alkyl,
substituted (phenoxy)C₀-C₂alkyl chosen from mono-, di-, and tri-substituted (phenoxy)C₀-C₂alkyl, wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
(heteroaryl)C₀-C₂alkyl, and
substituted (heteroaryl)C₀-C₂alkyl chosen from mono-, di-, and tri-substituted (heteroaryl)C₀-C₂alkyl, wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆alkylamino, di-C₁-C₆alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl;
Z₁ is chosen from 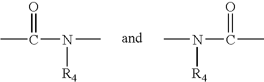
wherein
R₄ is chosen from
hydrogen,
C₁-C₆alkyl,
substituted C₁-C₆alkyl chosen from C₁-C₆alkyl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
C₃-C₇cylcoalkyl,
substituted C₃-C₇cycloalkyl chosen from C₃-C₇cycloalkyl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
heterocycloalkyl,
substituted heterocycloalkyl chosen from heterocycloalkyl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
phenyl,
substituted phenyl chosen from phenyl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
heteroaryl, and
substituted heteroaryl chosen from heteroaryl substituted by 1 or more substituents, wherein the substituents are chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl;
Q is chosen from phenylene and pyridylidene; and
R₃ is chosen from
hydrogen,
halo,
C₁-C₇ alkyl,
heterocycloalkyl,
substituted heterocycloalkyl chosen from mono-, di-, and tri-substituted heterocycloalkyl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
C₃-C₇cycloalkyl,
substituted C₃-C₇cycloalkyl chosen from mono-, di-, and tri-substituted C₃-C₇cycloalkyl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
heteroaryl, and
substituted heteroaryl chosen from mono-, di-, and tri-substituted heteroaryl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl.

2. At least one chemical entity of claim 1, wherein A is 0.

3. At least one chemical entity of claim 1, wherein A is 1.

4. At least one chemical entity of claim 1, wherein
R₁ is chosen from
phenyl substituted with a substituent chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁, chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a substituent chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₇-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

5. At least one chemical entity of claim 4, wherein
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH,
wherein R₁ is optionally substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

6. At least one chemical entity of claim 5, wherein
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH, wherein
R₁ is optionally further mono-, di-, or tri-substituted with groups independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

7. At least one chemical entity of claim 6, wherein
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH, wherein
R₁ is optionally further mono-, di-, or tri-substituted with groups independently chosen from hydroxy, cyano, halo, C₁-C₂alkyl, and C₁-C₂alkoxy.

8. At least one chemical entity of claim 4, wherein
R₁ is chosen from
phenyl substituted with a group chosen from
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

9. At least one chemical entity of claim 8, wherein
R₁ is chosen from
phenyl substituted with a group chosen from
C₁-C₆alkyl,
substituted C₁-C₆alkyl chosen from mono-, di-, and tri-substituted C₁-C₆alkyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
C₁-C₆alkoxy, and
substituted C₁-C₆alkoxy chosen from mono-, di-, and tri-substituted C₁-C₆alkoxy, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido, and
pyridyl substituted with a group chosen from
C₁-C₆alkyl,
substituted C₁-C₆alkyl chosen from mono-, di-, and tri-substituted C₁-C₆alkyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
C₁-C₆alkoxy, and
substituted C₁-C₆alkoxy chosen from mono-, di-, and tri-substituted C₁-C₆alkoxy, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

10. At least one chemical entity of claim 9, wherein
R₁ is chosen from
phenyl substituted with a group chosen from C₁-C₆hydroxyalkyl, C₁-C₆hydroxyalkoxy, (mono-C₁-C₆alkylamino)C₁-C₆alkoxy, (di-C₁-C₆alkylamino)C₁-C₆alkoxy, (C₁-C₆alkoxy)(C₁-C₆alkylamino)C₀-C₆alkyl, and (C₁-C₆alkoxy)(C₁-C₆alkoxy)C₁-C₆alkoxy, and
pyridyl substituted with a group chosen from C₁-C₆hydroxyalkyl, C₁-C₆hydroxyalkoxy, (mono-C₁-C₆alkylamino)C₁-C₆alkoxy, (di-C₁-C₆alkylamino)C₁-C₆alkoxy, (C₁-C₆alkoxy)(C₁-C₆alkylamino)C₀-C₆alkyl, and (C₁-C₆alkoxy)(C₁-C₆alkoxy)C₁-C₆alkoxy,
wherein R₁ is optionally further mono-, di-, or tri-substituted with substituents independently chosen from hydroxy, cyano, halo, C₁-C₂alkyl, and C₁-C₂alkoxy.

11. At least one chemical entity of claim 4, wherein
R₁ is chosen from
phenyl substituted with a group chosen from
pyrrolidinyl,
substituted pyrrolidinyl chosen from mono-, di- and tri-substituted pyrrolidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from hhalo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine,
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
pyrrolidinyl,
substituted pyrrolidinyl chosen from mono-, di- and tri-substituted pyrrolidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

12. At least one chemical entity of claim 11, wherein
R₁ is chosen from
phenyl substituted with a group chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy.

13. At least one chemical entity of claim 4, wherein
R₁ is chosen from
phenyl substituted with a group chosen from
—C₁-C₆alkylene(C═O)OR₁₀,
substituted —C₁-C₆alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylene(C═O)NR₁₀R₁₁,
substituted —C₀-C₆alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂, and
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from
—C₁-C₆alkylene(C═O)OR₁₀,
substituted —C₁-C₆alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylene(C═O)NR₁₀R₁₁,
substituted —C₀-C₆alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂, and
substituted —C₀-C₆alkylNR₁₀(C═O)NR₁₁R₁₂ chosen from mono-, di-, and tri-substituted C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl, and
wherein R₁₀, R₁₁, and R₁₂ are independently chosen from hydrogen, hydroxy, C₁-C₆alkyl, C₁-C₆alkoxy, C₃-C₇cycloalkyl, and heterocycloalkyl.

14. At least one chemical entity of claim 13, wherein
R₁ is chosen from
phenyl substituted with a group chosen from
—C₁-C₄alkylene(C═O)OR₁₀,
substituted —C₁-C₄alkylene(C═O)OR₁₀ chosen from mono-, di-, and tri-substituted —C₁-C₄alkylene(C═O)OR₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylene(C═O)NR₁₀R₁₁,
substituted —C₀-C₄alkylene(C═O)NR₁₀R₁₁ chosen from mono-, di-, and tri-substituted —C₀-C₄alkylene(C═O)NR₁₀R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₁-C₄alkylNR₁₀(SO₂)R₁₁,
substituted —C₁-C₄alkylNR₁₀(SO₂)R₁₁ chosen from mono-, di-, and tri-substituted —C₁-C₄alkylNR₁₀(SO₂)R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylNR₁₀(C═O)R₁₁,
substituted —C₀-C₄alkylNR₁₀(C═O)R₁₁, chosen from mono-, di-, and tri-substituted —C₀-C₄alkylNR₁₀(C═O)R₁₁, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkyl(SO₂)R₁₀,
substituted —C₀-C₄alkyl(SO₂)R₁₀ chosen from mono-, di-, and tri-substituted —C₀-C₄alkyl(SO₂)R₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylNR₁₀(C═O)NR₁₁R₁₂, and
substituted —C₀-C₄alkylNR₁₀(C═O)NR₁₁R₁₂ chosen from mono-, di-, and tri-substituted —C₀-C₄alkylNR₁₀(C═O)NR₁₁R₁₂, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
—C₁-C₄alkylene(C═O)OR₁₀,
substituted —C₁-C₄alkylene(C═O)OR₁₀ chosen from mono-, di-, and tri-substituted —C₁-C₄alkylene(C═O)OR₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylene(C═O)NR₁₀R₁₁,
substituted —C₀-C₄alkylene(C═O)NR₁₀R₁₁ chosen from mono-, di-, and tri-substituted —C₀-C₄alkylene(C═O)NR₁₀R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₁-C₄alkylNR₁₀(SO₂)R₁₁,
substituted —C₁-C₄alkylNR₁₀(SO₂)R₁₁ chosen from mono-, di-, and tri-substituted —C₁-C₄alkylNR₁₀(SO₂)R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylNR₁₀(C═O)R₁₁,
substituted —C₀-C₄alkylNR₁₀(C═O)R₁₁ chosen from mono-, di-, and tri-substituted —C₀-C₄alkylNR₁₀(C═O)R₁₁, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkyl(SO₂)R₁₀,
substituted —C₀-C₄alkyl(SO₂)R₁₀ chosen from mono-, di-, and tri-substituted —C₀-C₄alkyl(SO₂)R₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylNR₁₀(C═O)NR₁₁R₁₂ and
substituted —C₀-C₄alkylNR₁₀(C═O)NR₁₁R₁₂ chosen from mono-, di-, and tri-substituted —C₀-C₄alkylNR₁₀(C═O)NR₁₁R₁₂, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy, and
wherein R₁₀, R₁₁, and R₁₂ are independently chosen from hydrogen, hydroxy, C₁-C₆alkyl, C₁-C₆alkoxy, C₃-C₁₀cycloalkyl, pyrrolidinyl, morpholinyl, thiomorpholinyl, piperazinyl, piperidinyl, and [1,4]diazepanyl.

15. At least one chemical entity of claim 4, wherein
R₁ is chosen from
phenyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

16. At least one chemical entity of claim 15, wherein
R₁ is chosen from
phenyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, and di-C₁-C₄alkylcarboxamido,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
G is chosen from
pyrrolidinyl,
substituted pyrrolidinyl chosen from mono-, di-, and tri-substituted pyrrolidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkokycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di-, and tri-substituted morpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di-, and tri-substituted thiomorpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di-, and tri-substituted piperazinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di-, and tri-substituted piperidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di-, and tri-substituted [1,4]diazepanyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di-, and tri-substituted phenyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di-, and tri-substituted imidazolyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di-, and tri-substituted 5,6-dihydro-8H-[1,2,4]triazolo[4,3-a]pyrazine wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
G is chosen from
pyrrolidinyl,
substituted pyrrolidinyl chosen from mono-, di-, and tri-substituted pyrrolidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di-, and tri-substituted morpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di-, and tri-substituted thiomorpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di-, and tri-substituted piperazinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di-, and tri-substituted piperidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di-, and tri-substituted [1,4]diazepanyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di-, and tri-substituted phenyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di-, and tri-substituted imidazolyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di-, and tri-substituted 5,6-dihydro-8H-[1,2,4]triazolo[4,3-a]pyrazine wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

17. At least one chemical entity of claim 16, wherein
R₁ is chosen from
phenyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
G is chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
G is chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy.

18. At least one chemical entity of claim 17, wherein
L is chosen from
phenyl substituted with a group chosen from —CH₂—, —(C═O)—, and —(CH₂)(C═O)—, and
pyridyl substituted with a group chosen from —CH₂—, —(C═O)—, and —(CH₂)(C═O)—, and
G is chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
octahydro-pyrido[1,2-a]pyrazin-2-yl, and
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₂alkyl, C₁-C₂alkoxy, C₂alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
wherein R₁ is optionally further mono-, di-, or tri-substituted with substituents independently chosen from hydroxy, halo, C₁-C₂alkyl, and C₁-C₂alkoxy.

19. At least one chemical entity of claim 1, which is chosen from compounds of Formula 2 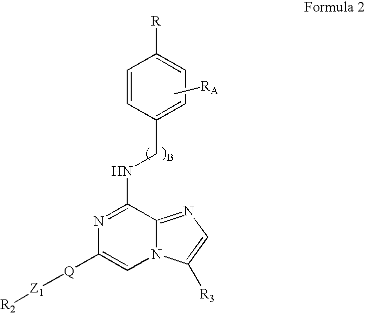
wherein
B is chosen from 0 and 1;
R is chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁, chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl, and
wherein R_{A} is optional and when present, can be present up to four times, and is independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

20. At least one chemical entity of claim 19, which is chosen from compounds of Formula 3

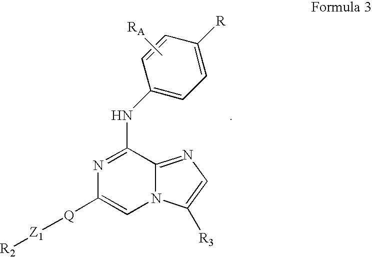

21. At least one chemical entity of claim 1, wherein
R₂ is chosen from
(phenyl)C₀-C₂alkyl,
substituted (phenyl)C₀-C₂alkyl chosen from mono-, di-, and tri-substituted (phenyl)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
(phenoxy)C₀-C₂alkyl,
substituted (phenoxy)C₀-C₂alkyl chosen from mono-, di-, and tri-substituted (phenoxy)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl,
(pyridyl)C₀-C₂alkyl, and
substituted (pyridyl)C₀-C₂alkyl chosen from mono-, di-, and tri-substituted (pyridyl)C₀-C₂alkyl wherein the substituents are independently chosen from hydroxy, nitro, cyano, amino, halo, —SO₂NH₂, oxo, —COOH, C₁-C₆ alkyl, C₂-C₆alkenyl, C₂-C₆alkynyl, C₁-C₆ alkoxy, C₂-C₆alkanoyl, C₁-C₆hydroxyalkyl, C₁-C₃haloalkyl, C₁-C₃haloalkoxy, C₁-C₆ alkylthio, (C₁-C₆ alkoxy)C₁-C₆ alkoxy, aminoC₁-C₆alkyl, mono-C₁-C₆ alkylamino, di-C₁-C₆ alkylamino, mono-C₁-C₆alkylcarboxamido, di-C₁-C₆alkylcarboxamido, C₁-C₆alkoxycarbonyl, phenyl, heteroaryl, and —(C═O)R₁₃ wherein R₁₃ is chosen from C₁-C₆alkyl, (C₃-C₇cycloalkyl)C₀-C₂alkyl, C₁-C₃haloalkyl, heterocycloalkyl, phenyl, and heteroaryl.

22. At least one chemical entity of claim 21, wherein
R₂ is chosen from
(phenyl)C₀-C₂alkyl,
substituted (phenyl)C₀-C₂alkyl chosen from mono-, di-, and tri-substituted (phenyl)C₀-C₂alkyl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl,
(phenoxy)C₀-C₂alkyl,
substituted (phenoxy)C₀-C₂alkyl chosen from mono-, di-, and tri-substituted (phenoxy)C₀-C₂alkyl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl,
(pyridyl)C₀-C₂alkyl, and
substituted (pyridyl)C₀-C₂alkyl chosen from mono-, di-, and tri-substituted (pyridyl)C₀-C₂alkyl wherein the substituents are independently chosen from hydroxy, cyano, amino, halo, —SO₂NH₂, oxo, —COOH, C₁-C₄ alkyl, C₁-C₄ alkoxy, C₂-C₄alkanoyl, C₁-C₄hydroxyalkyl, C₁-C₂haloalkyl, C₁-C₂haloalkoxy, mono-C₁-C₆alkylamino, di-C₁-C₆alkylamino, mono-C₁-C₆alkylcarboxamido, di-C₁-C₆alkylcarboxamido, C₁-C₄alkoxycarbonyl, phenyl, and imidazolyl.

23. At least one chemical entity of claim 1, wherein
Z₁ is chosen from 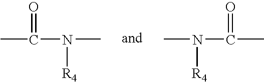
and
R₄ is chosen from hydrogen, C₁-C₆alkyl, and C₃-C₇cycloalkyl.

24. At least one chemical entity of claim 23, wherein R₄ is chosen from hydrogen and methyl.

25. At least one chemical entity of claim 1, which is chosen from compounds of Formula 4

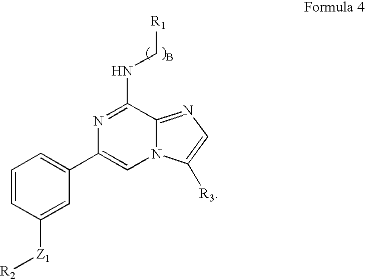

26. At least one chemical entity of claim 25, which is chosen from compounds of Formula 5 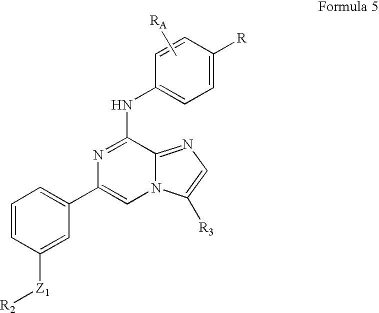
wherein
R is chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is
C₁-C₂alkyl,
substituted C₁-C₂alkyl chosen from mono-, di-, and tri-substituted C₁-C₂alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R_{A} is optional and when present, can be present up to four times, and is independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl, and
wherein R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl.

27. At least one chemical entity of claim 1, wherein R₃ is chosen from hydrogen and methyl.

28. At least one chemical entity chosen from compounds of Formula 6 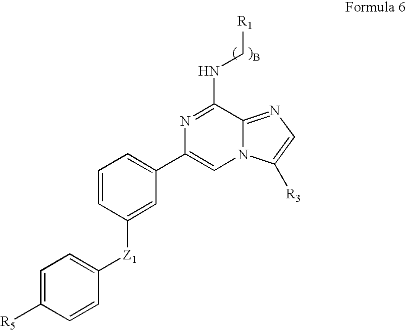
and pharmaceutically acceptable salts, solvates, crystal forms, chelates, non-covalent complexes, prodrugs, and mixtures thereof, wherein
B is chosen from 0 and 1;
R₁ is chosen from
phenyl,
phenyl substituted with a group chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁, chosen from mono-, di-, and tri-substituted —C₁-C₆alkylNR₁₀(SO₂)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino; mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
heteroaryl substituted with a group chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁, chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂ and
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl, and
wherein R₁ is further optionally substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl;
R₃ is chosen from
hydrogen,
halo,
C₁-C₇ alkyl,
heterocycloalkyl,
substituted heterocycloalkyl chosen from mono-, di-, and tri-substituted heterocycloalkyl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
C₃-C₇cycloalkyl,
substituted C₃-C₇cycloalkyl chosen from mono-, di-, and tri-substituted C₃-C₇cycloalkyl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl,
heteroaryl, and
substituted heteroaryl chosen from mono-, di-, and tri-substituted heteroaryl, wherein the substituents are independently chosen from hydroxy, nitro, cyano, amino, halo, —SO₂NH₂, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆-alkoxy)C₁-C₆ alkoxy, mono-C₁-C₆alkylamino, di-C₁-C₆alkylamino, aminoC₁-C₆ alkyl, and —C(O)R₁₄ wherein R₁₄ is chosen from C₁-C₆ alkyl, C₁-C₃haloalkyl, and phenyl;
Z₁ is chosen from 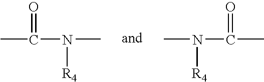
wherein
R₄ is chosen from
hydrogen,
C₁-C₆alkyl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
C₃-C₇cycloalkyl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
heterocycloalkyl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl,
phenyl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl, and
heteroaryl, which is optionally substituted with 1 or more substituents independently chosen from hydroxy, nitro, cyano, amino, halo, oxo, C₁-C₆ alkyl, C₂-C₆alkenyl, C₂-C₆alkynyl, C₁-C₆ alkoxy, C₁-C₃haloalkyl, C₁-C₃haloalkoxy, C₁-C₆ alkylthio, (C₁-C₆ alkoxy)C₁-C₆ alkoxy, aminoC₁-C₆ alkyl, mono-C₁-C₆ alkylamino, di-C₁-C₆ alkylamino, and —C(O)R₁₄ wherein R₁₄ is chosen from C₁-C₆ alkyl, C₁-C₃haloalkyl, and phenyl; and
R₅ is chosen from isopropyl and t-butyl.

29. At least one chemical entity of claim 28, wherein A is 0.

30. At least one chemical entity of claim 28, wherein A is 1.

31. At least one chemical entity of claim 28, wherein
R₁ is chosen from
phenyl,
phenyl substituted with a group chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁, chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
pyridyl, and
pyridyl substituted with
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁, chosen from mono-, di-, and tri-substituted —C₁-C₆alkylNR₁₀(SO₂)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂, and
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

32. At least one chemical entity of claim 31, wherein
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

33. At least one chemical entity of claim 32, wherein
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

34. At least one chemical entity of claim 33, wherein
R₁ is chosen from
phenyl substituted with a group chosen from hydroxy, —CHO, —COOH, —CONH₂, and —CONHOH, and
pyridyl substituted with a group chosen from hydroxy, —CHO, —COOH, —CONH₂, and —CONHOH,
wherein R₁ is optionally further mono-, di-, or tri-substituted with substituents independently chosen from hydroxy, cyano, halo, C₁-C₂alkyl, and C₁-C₂alkoxy.

35. At least one chemical entity of claim 31, wherein
R₁ is chosen from
phenyl substituted with a group chosen from
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

36. At least one chemical entity of claim 35, wherein
R₁ is chosen from
phenyl substituted with a group chosen from
C₁-C₆alkyl,
substituted C₁-C₆alkyl chosen from mono-, di-, and tri-substituted C₁-C₆alkyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
C₁-C₆alkoxy, and
substituted C₁-C₆alkoxy chosen from mono-, di-, and tri-substituted C₁-C₆alkoxy, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido, and
pyridyl substituted with a group chosen from
C₁-C₆alkyl,
substituted C₁-C₆alkyl chosen from mono-, di-, and tri-substituted C₁-C₆alkyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
C₁-C₆alkoxy, and
substituted C₁-C₆alkoxy chosen from mono-, di-, and tri-substituted C₁-C₆alkoxy, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₆alkylamino, di-C₁-C₆alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

37. At least one chemical entity of claim 36, wherein
R₁ is chosen from
phenyl substituted with a group chosen from C₁-C₆hydroxyalkyl, C₁-C₆hydroxyalkoxy, (mono-C₁-C₆alkylamino)C₁-C₆alkoxy, (di-C₁-C₆alkylamino)C₁-C₆alkoxy, (C₁-C₆alkoxy)(C₁-C₆alkylamino)C₀-C₆alkyl, and (C₁-C₆alkoxy)(C₁-C₆alkoxy)C₁-C₆alkoxy, and
pyridyl, substituted with a group chosen from C₁-C₆hydroxyalkyl, C₁-C₆hydroxyalkoxy, (mono-C₁-C₆alkylamino)C₁-C₆alkoxy, (di-C₁-C₆alkylamino)C₁-C₆alkoxy, (C₁-C₆alkoxy)(C₁-C₆alkylamino)C₀-C₆alkyl, and (C₁-C₆alkoxy)(C₁-C₆alkoxy)C₁-C₆alkoxy,
wherein R₁ is optionally further mono-, di-, or tri-substituted with substituents independently chosen from hydroxy, cyano, halo, C₁-C₂alkyl, and C₁-C₂alkoxy.

38. At least one chemical entity of claim 31, wherein
R₁ is chosen from
phenyl substituted with a group chosen from
pyrrolidinyl,
substituted pyrrolidinyl chosen from mono-, di- and tri-substituted pyrrolidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
pyrrolidinyl,
substituted pyrrolidinyl chosen from mono-, di- and tri-substituted pyrrolidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

39. At least one chemical entity of claim 38, wherein
R₁ is chosen from
phenyl substituted with a group chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkyl amino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy.

40. At least one chemical entity of claim 31, wherein
R₁ is chosen from
phenyl substituted with a group chosen from
—C₁-C₆alkylene(C═O)OR₁₀,
substituted —C₁-C₆alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylene(C═O)NR₁₀R₁₁,
substituted —C₀-C₆alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁, chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂, and
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from
—C₁-C₆alkylene(C═O)OR₁₀,
substituted —C₁-C₆alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylene(C═O)NR₁₀R₁₁,
substituted —C₀-C₆alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂ and
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁₀, R₁₁, and R₁₂ are independently chosen from hydrogen, hydroxy, C₁-C₆alkyl, C₁-C₆alkoxy, C₃-C₇cycloalkyl, and heterocycloalkyl, and
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

41. At least one chemical entity of claim 40, wherein
R₁ is chosen from
phenyl substituted with a group chosen from
—C₁-C₄alkylene(C═O)OR₁₀,
substituted —C₁-C₄alkylene(C═O)OR₁₀ chosen from mono-, di-, and tri-substituted —C₁-C₄alkylene(C═O)OR₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylene(C═O)NR₁₀R₁₁,
substituted —C₀-C₄alkylene(C═O)NR₁₀R₁₁ chosen from mono-, di-, and tri-substituted —C₀-C₄alkylene(C═O)NR₁₀R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₁-C₄alkylNR₁₀(SO₂)R₁₁,
substituted —C₁-C₄alkylNR₁₀(SO₂)R₁₁ chosen from mono-, di-, and tri-substituted —C₁-C₄alkylNR₁₀(SO₂)R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylNR₁₀(C═O)R₁₁,
substituted —C₀-C₄alkylNR₁₀(C═O)R₁₁ chosen from mono-, di-, and tri-substituted —C₀-C₄alkylNR₁₀(C═O)R₁₁, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkyl(SO₂)R₁₀,
substituted —C₀-C₄alkyl(SO₂)R₁₀ chosen from mono-, di-, and tri-substituted —C₀-C₄alkyl(SO₂)R₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylNR₁₀(C═O)NR₁₁R₁₂ and
substituted —C₀-C₄alkylNR₁₀(C═O)NR₁₁R₁₂ chosen from mono-, di-, and tri-substituted —C₀-C₄alkylNR₁₀(C═O)NR₁₁R₁₂, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with a group chosen from
—C₁-C₄alkylene(C═O)OR₁₀,
substituted —C₁-C₄alkylene(C═O)OR₁₀ chosen from mono-, di-, and tri-substituted —C₁-C₆alkylene(C═O)OR₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylene(C═O)NR₁₀R₁₁,
substituted —C₀-C₄alkylene(C═O)NR₁₀R₁₁ chosen from mono-, di-, and tri-substituted —C₀-C₆alkylene(C═O)NR₁₀R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₁-C₄alkylNR₁₀(SO₂)R₁₁,
substituted —C₁-C₄alkylNR₁₀(SO₂)R₁₁ chosen from mono-, di-, and tri-substituted —C₁-C₄alkylNR₁₀(SO₂)R₁₁ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylNR₁₀(C═O)R₁₁,
substituted —C₀-C₄alkylNR₁₀(C═O)R₁₁ chosen from mono-, di-, and tri-substituted —C₀-C₄alkylNR₁₀(C═O)R₁₁, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkyl(SO₂)R₁₀,
substituted —C₀-C₄alkyl(SO₂)R₁₀ chosen from mono-, di-, and tri-substituted —C₀-C₄alkyl(SO₂)R₁₀ wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—C₀-C₄alkylNR₁₀(C═O)NR₁₁R₁₂ and
substituted —C₀-C₄alkylNR₁₀(C═O)NR₁₁R₁₂ chosen from mono-, di-, and tri-substituted —C₀-C₄alkylNR₁₀(C═O)NR₁₁R₁₂, wherein the substituents are independently chosen from halo, hydroxy, amino, oxo, —COOH, —CONH₂, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁₀, R₁₁, and R₁₂ are independently chosen from hydrogen, hydroxy, C₁-C₆alkyl, C₁-C₆alkoxy, C₃-C₁₀cycloalkyl, pyrrolidinyl, morpholinyl, thiomorpholinyl, piperazinyl, piperidinyl, and [1,4]diazepanyl, and
wherein R₁ is optionally further mono-, di-, or tri-substituted with substituents independently chosen from hydroxy, cyano, halo, C₁-C₂alkyl, and C₁-C₂alkoxy.

42. At least one chemical entity of claim 31, wherein
R₁ is chosen from
phenyl substituted with a group chosen from
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
pyridyl substituted with a group chosen from
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R₁ is optionally further substituted with 1 or more substituents independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

43. At least one chemical entity of claim 42, wherein
R₁ is chosen from
phenyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
G is chosen from
pyrrolidinyl,
substituted pyrrolidinyl chosen from mono-, di-, and tri-substituted pyrrolidinyl wherein the substituents are independently chosen from halo, hydroxy, cyano, amino, oxo, —COOH, —CONH₂, C₁-C₄alkyl, C₁-C₆alkoxy, C₂-C₄alkanoyl C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di-, and tri-substituted morpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di-, and tri-substituted thiomorpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di-, and tri-substituted piperazinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di-, and tri-substituted piperidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkyl amino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di-, and tri-substituted [1,4]diazepanyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di-, and tri-substituted phenyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di-, and tri-substituted imidazolyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine,
substituted 5,6-dihydro-8H-[1,2,4]triazolo[4,3-a]pyrazine chosen from mono-, di-, and tri-substituted 5,6-dihydro-8H-[1,2,4]triazolo[4,3-a]pyrazine wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—(C═O)—,
—(C₁-C₂alkyl)(C═O)—, and
substituted —(C₁-C₂alkyl)(C═O)— chosen from mono-, di-, and tri-substituted —(C—C₂alkyl)(C═O)— wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
G is chosen from
pyrrolidinyl,
substituted pyrrolidinyl chosen from mono-, di-, and tri-substituted pyrrolidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di-, and tri-substituted morpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di-, and tri-substituted thiomorpholinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di-, and tri-substituted piperazinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di-, and tri-substituted piperidinyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di-, and tri-substituted [1,4]diazepanyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di-, and tri-substituted phenyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di-, and tri-substituted imidazolyl wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di-, and tri-substituted 5,6-dihydro-8H-[1,2,4]triazolo[4,3-a]pyrazine wherein the substituents are independently chosen from halo, hydroxy, cyano, amino, oxo, —COOH, —CONH₂, C₁-C₄alkyl, C₁-C₆alkoxy, C₂-C₄alkanoyl, C₁-C₂haloalkyl, C₁-C₂haloalkoxy, C₁-C₄alkoxycarbonyl, mono-C₁-C₄alkyl amino, di-C₁-C₄alkyl amino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₄alkyl, C₂-C₄alkanoyl, C₁-C₂haloalkyl, C₁-C₂haloalkoxy, C₁-C₄alkoxy, mono-C₁-C₄alkylamino, and di-C₁-C₄alkylamino.

44. At least one chemical entity of claim 43, wherein
R₁ is chosen from
phenyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
G is chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
pyridyl substituted with -L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, oxo, —COOH, —CONH₂, C₁-C₄alkyl, C₁-C₆alkoxy, C₂-C₄alkanoyl C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
—(C═O)—,
—(C₁-C₂alkyl)(C═O)—, and
substituted —(C₁-C₂alkyl)(C═O)— chosen from mono-, di-, and tri-substituted —(C₁-C₂alkyl)(C═O)— wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido, and
G is chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
[1,4]diazepanyl,
substituted [1,4]diazepanyl chosen from mono-, di- and tri-substituted [1,4]diazepanyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
octahydro-pyrido[1,2-a]pyrazin-2-yl,
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
phenyl,
substituted phenyl chosen from mono-, di- and tri-substituted phenyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
imidazolyl,
substituted imidazolyl chosen from mono-, di- and tri-substituted imidazolyl, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
5,6-dihydro-8H-[1,2,4]triazolo[4,3-a]pyrazine, and
substituted 5,6-dihydro-8H-[1,2,4]triazolo[4,3-a]pyrazine chosen from mono-, di- and tri-substituted 5,6-dihydro-8H-[1,2,4]triazolo[4,3-a]pyrazine, wherein the substituents are independently chosen from halo, hydroxy, oxo, —COOH, —CONH₂, C₁-C₄alkyl, C₁-C₆alkoxy, C₂-C₄alkanoyl, C₁-C₄alkoxycarbonyl, mono-C₁-C₄alkylamino, di-C₁-C₄alkylamino, mono-C₁-C₄alkylcarboxamido, and di-C₁-C₄alkylcarboxamido,
wherein R₁ is optionally further mono-, di-, or tri-substituted with substituents independently chosen from hydroxy, cyano, halo, C₁-C₂alkyl, and C₁-C₂alkoxy.

45. At least one chemical entity of claim 44, wherein
L is chosen from
phenyl substituted with a group chosen from —CH₂—, —(C═O)—, and —(CH₂)(C═O)— and
pyridyl substituted with a group chosen from —CH₂—, —(C═O)—, and —(CH₂)(C═O)— and
G is chosen from
pyrrolidinyl,
substituted pyrrolidinyl chosen from mono-, di- and tri-substituted pyrrolidinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
morpholinyl,
substituted morpholinyl chosen from mono-, di- and tri-substituted morpholinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
thiomorpholinyl,
substituted thiomorpholinyl chosen from mono-, di- and tri-substituted thiomorpholinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
piperazinyl,
substituted piperazinyl chosen from mono-, di- and tri-substituted piperazinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
piperidinyl,
substituted piperidinyl chosen from mono-, di- and tri-substituted piperidinyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
[1,4]diazepanyl, and
substituted [1,4]diazepanyl chosen from mono-, di- and tri-substituted [1,4]diazepanyl, wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido,
octahydro-pyrido[1,2-a]pyrazin-2-yl, and
substituted octahydro-pyrido[1,2-a]pyrazin-2-yl chosen from mono-, di-, and tri-substituted octahydro-pyrido[1,2-a]pyrazin-2-yl wherein the substituents are independently chosen from halo, hydroxy, oxo, —COOH, —CONH₂, C₁-C₂alkyl, C₁-C₂alkoxy, C₂-alkanoyl, C₁-C₂alkoxycarbonyl, mono-C₁-C₂alkylamino, di-C₁-C₂alkylamino, mono-C₁-C₂alkylcarboxamido, and di-C₁-C₂alkylcarboxamido.

46. At least one chemical entity of claim 28, which is chosen from compounds of Formula 7 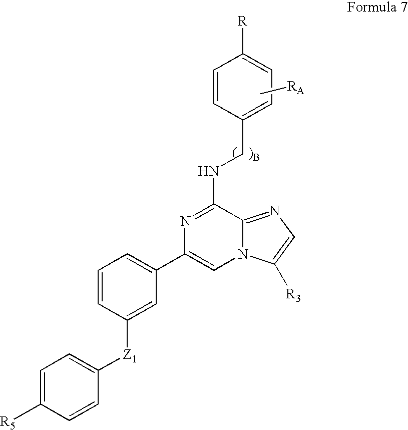
wherein
B is chosen from 0 and 1;
R is chosen from
hydroxy,
—CHO,
—COOH,
—CONH₂,
—CONHOH,
C₂-C₆ alkenyl,
substituted C₂-C₆ alkenyl chosen from mono-, di-, and tri-substituted C₂-C₆ alkenyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₂-C₆ alkynyl,
substituted C₂-C₆ alkynyl chosen from mono-, di-, and tri-substituted C₂-C₆ alkynyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkyl,
substituted C₁-C₆alkyl chosen from mono-, di-, and tri-substituted C₁-C₆alkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₁-C₆alkoxy,
substituted C₁-C₆alkoxy chosen from mono-, di-, and tri-substituted C₁-C₆alkoxy wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heterocycloalkyl,
substituted heterocycloalkyl chosen from mono-, di-, and tri-substituted heterocycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl,
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₁-C₆alkylNR₁₀(SO₂)R₁₁,
substituted —C₁-C₆alkylNR₁₀(SO₂)R₁₁ chosen from mono-, di-, and tri-substituted —C₁-C₆alkylNR₁₀(SO₂)R₁₁ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)R₁₁,
substituted —C₀-C₆alkylNR₁₀(C═O)R₁₁ chosen from mono-, di-, and tri-substituted —C₀-C₆alkylNR₁₀(C═O)R₁₁, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkyl(SO₂)R₁₀,
substituted —C₀-C₆alkyl(SO₂)R₁₀ chosen from mono-, di-, and tri-substituted —C₀-C₆alkyl(SO₂)R₁₀ wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—C₀-C₆alkylNR₁₀(C═O)NR₁₁R₁₂,
substituted —C₀-C₆alkylNR₁₀(C═O)NR₁₁R₁₂ chosen from mono-, di-, and tri-substituted —C₀-C₆alkylNR₁₀(C═O)NR₁₁R₁₂, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
where R₁₀, R₁₁, and R₁₂ are independently chosen from hydrogen, hydroxy, C₁-C₆alkyl, C₁-C₆alkoxy, C₃-C₁₀cycloalkyl, and heterocycloalkyl, and
-L-G, where
L is chosen from
C₁-C₂alkylene,
substituted C₁-C₂alkylene chosen from mono-, di-, and tri-substituted C₁-C₂alkylene wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₀-C₂alkylene-O—,
substituted C₀-C₂alkylene-O— chosen from mono-, di-, and tri-substituted C₀-C₂alkylene-O— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
—(C═O)—,
—(C₁-C₂alkylene)(C═O)—, and
substituted —(C₁-C₂alkylene)(C═O)— chosen from mono-, di-, and tri-substituted —(C₁-C₂alkylene)(C═O)— wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl, and
and G is chosen from
heterocycloalkyl,
substituted heterocycloalkyl chosen from mono-, di-, and tri-substituted heterocycloalkyl, wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
C₃-C₇cycloalkyl,
substituted C₃-C₇cycloalkyl chosen from mono-, di-, and tri-substituted C₃-C₇cycloalkyl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
aryl,
substituted aryl chosen from mono-, di-, and tri-substituted aryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
heteroaryl, and
substituted heteroaryl chosen from mono-, di-, and tri-substituted heteroaryl wherein the substituents are independently chosen from halo, hydroxy, amino, oxo, —COOH, —CONH₂, C₁-C₄alkyl, C₂-C₄alkenyl, C₂-C₄alkynyl, C₁-C₆alkoxy, C₂-C₄alkanoyl, C₁-C₄alkylthio, C₁-C₂haloalkyl, C₁-C₂haloalkoxy, C₁-C₄alkoxycarbonyl, mono-C₁-C₄alkylamino, di-C₁-C₄alkylamino, mono-C₁-C₄alkylcarboxamido, di-C₁-C₄alkylcarboxamido, and phenyl,
wherein R_{A} is optional and when present, can be present up to four times, and is independently chosen from hydroxy, nitro, cyano, amino, —SO₂NH₂, halo, C₁-C₆ alkyl, C₂-C₆alkanoyl, C₁-C₄haloalkyl, C₁-C₄haloalkoxy, C₁-C₆ alkylthio, C₃-C₇cycloalkyl, (C₁-C₆ alkoxy)C₀-C₆alkyl, (mono-C₁-C₆ alkylamino)C₀-C₆ alkyl, (di-C₁-C₆ alkylamino)C₀-C₆ alkyl, and C₁-C₆alkoxycarbonyl.

47. At least one chemical entity of claim 46, which is chosen from compounds of Formula 8

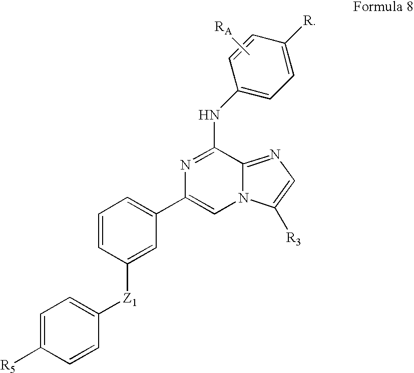

48. At least one chemical entity of claim 47, which is chosen from compounds of Formula 9

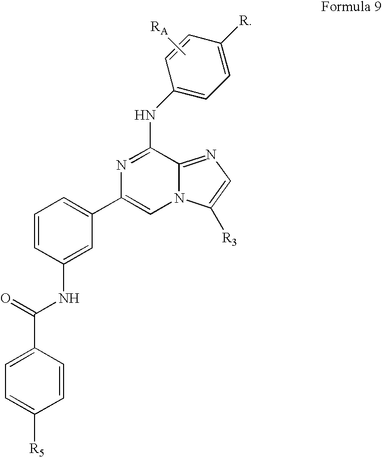

49. At least one chemical entity of claim 28, wherein R₃ is chosen from hydrogen and methyl.

50. At least one chemical entity of claim 1, wherein the at least one chemical entity exhibits an IC₅₀ of 1 micromolar or less in an in vitro biochemical assay of Btk activity.

51. At least one chemical entity of claim 50, wherein the at least one chemical entity exhibits an IC₅₀ of 100 nanomolar or less in an in vitro biochemical assay of Btk activity.

52. At least one chemical entity of claim 52, wherein the at least one chemical entity-exhibits an IC₅₀ of 10 nanomolar or less in an in vitro biochemical assay of Btk activity.

53. At least one chemical entity of claim 1, wherein the at least one chemical entity exhibits an IC₅₀ of 10 micromolar or less in an assay for inhibition of B-cell proliferation.

54. At least one chemical entity of claim 53, wherein the at least one chemical entity exhibits an IC₅₀ of 1 micromolar or less in an assay for inhibition of B-cell proliferation.

55. At least one chemical entity of claim 54, wherein the at least one chemical entity exhibits an IC₅₀ of 500 nanomolar or less in an assay for inhibition of B-cell proliferation.

56. At least one chemical entity of claim 1, wherein the at least one chemical entity exhibits an IC₅₀ value in an assay for inhibition of T-cell proliferation that is at least 3-fold greater than an IC₅₀ value that the at least one chemical entity exhibits in an assay for inhibition of B-cell proliferation.

57. At least one chemical entity of claim 56, wherein the at least one chemical entity exhibits an IC₅₀ value in an assay for inhibition of T-cell proliferation that is at least 5-fold greater than an IC₅₀ value that the at least one chemical entity exhibits in an assay for inhibition of B-cell proliferation.

58. At least one chemical entity of claim 57, wherein the at least one chemical entity exhibits an IC₅₀ value in an assay for inhibition of T-cell proliferation that is at least 10-fold greater than an IC₅₀ value the that at least one chemical entity exhibits in an assay for inhibition of B-cell proliferation.

59. A pharmaceutical composition, comprising at least one chemical entity of claim 1, together with at least one pharmaceutically acceptable carrier or excipient.

60. A pharmaceutical composition of claim 59, wherein the composition is formulated in a form chosen from injectable fluids, aerosols, creams, gels, tablets, pills, capsules, syrups, ophthalmic solutions, and transdermal patches.

61. A packaged pharmaceutical composition, comprising a pharmaceutical composition of claim 59; and instructions for using the composition to treat a patient suffering from a disease responsive to Btk inhibition.

62. The packaged pharmaceutical composition of claim 61 wherein the disease responsive to Btk inhibition is cancer.

63. The packaged pharmaceutical composition of claim 61 wherein the disease responsive to Btk inhibition is chosen from autoimmune diseases, inflammatory diseases, and acute inflammatory reactions.

64. A method for treating a patient having a disease responsive to inhibition of Btk activity, comprising administering to the patient an effective amount of at least one chemical entity of claim 1.

65. The method of claim 64 wherein the patient is a human.

66. The method of claim 64 wherein the patient is chosen from cats and dogs.

67. The method of claim 64 wherein the disease responsive to Btk inhibition is cancer.

68. The method of claim 64 wherein the disease responsive to Btk inhibition is administered by a method chosen from intravenously, intramuscularly, and parenterally.

69. The method of claim 64 wherein at least one chemical entity is administered orally.

70. The method of claim 64 wherein the at least one chemical entity is administered intravenously, intramuscularly, or parenterally.

71. A method for increasing sensitivity of cancer cells to chemotherapy, comprising administering to a patient undergoing chemotherapy with a chemotherapeutic agent an amount of at least one chemical entity of claim 1, sufficient to increase the sensitivity of cancer cells to the chemotherapeutic agent.

72. A method of reducing medication error and enhancing therapeutic compliance of a patient being treated for a disease responsive to Btk inhibition, the method comprising providing a packaged pharmaceutical preparation of claim 61 wherein the instructions additionally include contraindication and adverse reaction information pertaining to the packaged pharmaceutical composition.

73. A method for increasing sensitivity of cancer cells to chemotherapy, comprising administering to a patient undergoing chemotherapy with a chemotherapeutic agent an amount of at least one chemical entity of claim 1, sufficient to increase the sensitivity of cancer cells to the chemotherapeutic agent.

74. A method for inhibiting ATP hydrolysis, the method comprising contacting cells expressing Btk with at least one chemical entity of claim 1 in an amount sufficient to detectably decrease the level of ATP hydrolysis in vitro.

75. The method of claim 74 wherein the cells are present in a mammal.

76. The method of claim 75 wherein the mammal is a human.

77. The method of claim 75 wherein the mammal is chosen from cats and dogs.

78. A method for determining the presence of Btk in a sample, comprising contacting the sample with at least one chemical entity of claim 1 under conditions that permit detection of Btk activity, detecting a level of Btk activity in the sample, and therefrom determining the presence or absence of Btk in the sample.

79. A method for inhibiting B-cell proliferation comprising contacting cells expressing Btk with at least one chemical entity, of claim 1, in an amount sufficient to detectably decrease Btk activity in vitro.

80. At least one one chemical entity chosen from
4-tert-Butyl-N-[3-(8-phenylamino-imidazo[1,2-a]pyrazin-6-yl)-phenyl]-benzamide;
4-Isopropyl-N-[3-(8-phenylamino-imidazo[1,2-a]pyrazin-6-yl)-phenyl]-benzamide;
4-tert-Butyl-N-{3-[8-(pyridin-3-ylamino)-imidazo[1,2-a]pyrazin-6-yl]-phenyl}-benzamide;
4-tert-Butyl-N-{3-[8-(4-methoxy-phenylamino)-imidazo[1,2-a]pyrazin-6-yl]-phenyl}-benzamide;
4-tert-Butyl-N-{3-[8-(pyridin-4-ylamino)-imidazo[1,2-a]pyrazin-6-yl]-phenyl}-benzamide;
4-tert-Butyl-N-{3-[8-(4-fluoro-phenylamino)-imidazo[1,2-a]pyrazin-6-yl]-phenyl}-benzamide;
4-tert-Butyl-N-{3-[8-(3-fluoro-phenylamino)-imidazo[1,2-a]pyrazin-6-yl]-phenyl}-benzamide;
4-{6-[3-(4-tert-Butyl-benzoylamino)-phenyl]-imidazo[1,2-a]pyrazin-8-ylamino}-benzoic acid;
4-tert-Butyl-N-{3-[8-(3-methoxy-phenylamino)-imidazo[1,2-a]pyrazin-6-yl]-phenyl}-benzamide;
4-tert-Butyl-N-{3-[8-(6-methoxy-pyridin-3-ylamino)-imidazo[1,2-a]pyrazin-6-yl]-phenyl}-benzamide;
3-{6-[3-(4-tert-Butyl-benzoylamino)-phenyl]-imidazo[1,2-a]pyrazin-8-ylamino}-benzoic acid;
4-{6-[3-(4-Isopropyl-benzoylamino)-phenyl]-imidazo[1,2-a]pyrazin-8-ylamino}-benzoic acid;
4-tert-Butyl-N-{3-[8-(4-cyano-phenylamino)-imidazo[1,2-a]pyrazin-6-yl]-phenyl}-benzamide;
4-{6-[3-(4-tert-Butyl-benzoylamino)-phenyl]-imidazo[1,2-a]pyrazin-8-ylamino}-benzamide;
4-tert-Butyl-N-{3-[8-(4-morpholin-4-yl-phenylamino)-imidazo[1,2-a]pyrazin-6-yl]-phenyl}-benzamide;
3-{6-[3-(4-tert-Butyl-benzoylamino)-phenyl]-imidazo[1,2-a]pyrazin-8-ylamino}-benzoic acid;
4-tert-Butyl-N-{3-[8-(3-fluoro-4-methoxy-phenylamino)-imidazo[1,2-a]pyrazin-6-yl]-phenyl}-benzamide;
4-{6-[3-(4-tert-Butyl-benzoylamino)-phenyl]-imidazo[1,2-a]pyrazin-8-ylamino}-benzoic N-Methyl amide;
4-tert-Butyl-N-{3-[8-(4-piperazin-1-yl-phenylamino)-imidazo[1,2-a]pyrazin-6-yl]-phenyl}-benzamide;
4-tert-Butyl-N-[3-(8-m-tolylamino-imidazo[1,2-a]pyrazin-6-yl)-phenyl]-benzamide;
(4-{6-[3-(4-tert-Butyl-benzoylamino)-phenyl]-imidazo[1,2-a]pyrazin-8-ylamino}-phenyl)-acetic acid methyl ester;
(4-{6-[3-(4-tert-Butyl-benzoylamino)-phenyl]-imidazo[1,2-a]pyrazin-8-ylamino}-phenyl)-acetic-acid;
4-tert-Butyl-N-(3-{8-[4-(piperazine-1-carbonyl)-phenylamino]-imidazo[1,2-a]pyrazin-6-yl}-phenyl)-benzamide;
4-tert-Butyl-N-(3-{8-[4-(2-methoxy-ethoxymethoxy)-phenylamino]-imidazo[1,2-a]pyrazin-6-yl}-phenyl)-benzamide;
4-tert-Butyl-N-{3-[8-(4-hydroxy-phenylamino)-imidazo[1,2-a]pyrazin-6-yl]-phenyl}-benzamide;
4-tert-Butyl-N-{3-[8-(6-piperazin-1-yl-pyridin-3-ylamino)-imidazo[1,2-a]pyrazin-6-yl]-phenyl}-benzamide;
4-tert-Butyl-N-(3-{8-[4-(4-methyl-piperazin-1-yl)-phenylamino]-imidazo[1,2-a]pyrazin-6-yl}-phenyl)-benzamide;
4-tert-Butyl-N-{3-[8-(4-[1,4]diazepan-1-yl-phenylamino)-imidazo[1,2-a]pyrazin-6-yl]-phenyl}-benzamide;
4-tert-Butyl-N-(3-{8-[4-(4-oxo-piperidin-1-yl)-phenylamino]-imidazo[1,2-a]pyrazin-6-yl}-phenyl)-benzamide;
(4-{6-[3-(4-tert-Butyl-benzoylamino)-phenyl]-imidazo[1,2-a]pyrazin-8-ylamino}-benzoylamino)-acetic acid methyl ester;
(4-{6-[3-(4-tert-Butyl-benzoylamino)-phenyl]-imidazo[1,2-a]pyrazin-8-ylamino}-benzoylamino)-acetic acid;
N-(3-{8-[4-(4-Acetyl-piperazin-1-yl)-phenylamino]-imidazo[1,2-a]pyrazin-6-yl}-phenyl)-4-tert-butyl-benzamide;
4-tert-Butyl-N-(3-{8-[4-(3-methyl-piperazin-1-yl)-phenylamino]-imidazo[1,2-a]pyrazin-6-yl}-phenyl)-benzamide;
4-{6-[3-(4-tert-Butyl-benzoylamino)-phenyl]-imidazo[1,2-a]pyrazin-8-ylamino}-benzoic hydroxyl amide;
4-tert-Butyl-N-{3-[8-(4-hydroxymethyl-phenylamino)-imidazo[1,2-a]pyrazin-6-yl]-phenyl}-benzamide;
4-tert-Butyl-N-{3-[8-(4-piperazin-1-ylmethyl-phenylamino)-imidazo[1,2-a]pyrazin-6-yl]-phenyl}-benzamide;
4-{6-[3-(4-Bromo-3-methyl-benzoylamino)-phenyl]-imidazo[1,2-a]pyrazin-8-ylamino}-benzoic acid;
4-tert-Butyl-N-{3-[8-(3-methyl-4-piperazin-1-yl-phenylamino)-imidazo[1,2-a]pyrazin-6-yl]-phenyl}-benzamide;
4-tert-Butyl-N-(3-{8-[4-(3-methyl-piperazin-1-yl)-phenylamino]-imidazo[1,2-a]pyrazin-6-yl}-phenyl)-benzamide;
4-tert-Butyl-N-{3-[8-(3-hydroxymethyl-phenylamino)-imidazo[1,2-a]pyrazin-6-yl]-phenyl}-benzamide;
4-tert-Butyl-N-(3-{8-[3-(3-methyl-piperazin-1-ylmethyl)-phenylamino]-imidazo[1,2-a]pyrazin-6-yl}-phenyl)-benzamide;
4-tert-Butyl-N-(3-{8-[3-(3-methyl-piperazin-1-ylmethyl)-phenylamino]-imidazo[1,2-a]pyrazin-6-yl}-phenyl)-benzamide;
4-tert-Butyl-N-(3-{8-[3-(3,5-dimethyl-piperazin-1-ylmethyl)-phenylamino]-imidazo[1,2-a]pyrazin-6-yl}-phenyl)-benzamide;
4-tert-Butyl-N-(3-{8-[4-(3,5-dimethyl-piperazin-1-yl)-phenylamino]-imidazo[1,2-a]pyrazin-6-yl}-phenyl)-benzamide;
4-({6-[3-(4-tert-Butyl-benzoylamino)-phenyl]-imidazo[1,2-a]pyrazin-8-ylamino}-methyl)-benzoic acid;
4-tert-Butyl-N-(3-{8-[4-(3-methyl-piperazin-1-yl)-phenylamino]-imidazo[1,2-a]pyrazin-6-yl}-phenyl)-benzamide;
4-(1-Hydroxy-1-methyl-ethyl)-N-[3-(8-phenylamino-imidazo[1,2-a]pyrazin-6-yl)-phenyl]-benzamide;
(3-{6-[3-(4-tert-Butyl-benzoylamino)-phenyl]-imidazo[1,2-a]pyrazin-8-ylamino}-phenyl)-acetic acid;
N-[3-(8-Phenylamino-imidazo[1,2-a]pyrazin-6-yl)-phenyl]-terephthalamic acid methyl ester;
N-[3-(8-Phenylamino-imidazo[1,2-a]pyrazin-6-yl)-phenyl]-terephthalamic acid;
2-(4-{6-[3-(4-tert-Butyl-benzoylamino)-phenyl]-imidazo[1,2-a]pyrazin-8-ylamino}-phenyl)-propionic acid ethyl ester;
2-(4-{6-[3-(4-tert-Butyl-benzoylamino)-phenyl]-imidazo[1,2-a]pyrazin-8-ylamino}-phenyl)-propionic acid;
4-{6-[3-(4-tert-Butyl-benzoylamino)-phenyl]-imidazo[1,2-a]pyrazin-8-ylamino}-2-methoxy-benzoic acid methyl ester;
4-{6-[3-(4-tert-Butyl-benzoylamino)-phenyl]-imidazo[1,2-a]pyrazin-8-ylamino}-2-methoxy-benzoic acid;
4-tert-Butyl-N-{3-[8-(3-methylcarbamoylmethyl-phenylamino)-imidazo[1,2-a]pyrazin-6-yl]-phenyl}-benzamide;
4-tert-Butyl-N-{3-[8-(4-methylcarbamoylmethyl-phenylamino)-imidazo[1,2-a]pyrazin-6-yl]-phenyl}-benzamide;
4-tert-Butyl-N-{3-[8-(4-cyclooctylcarbamoylmethyl-phenylamino)-imidazo[1,2-a]pyrazin-6-yl]-phenyl}-benzamide;
4-tert-Butyl-N-[3-(8-{4-[(diisopropylcarbamoyl)-methyl]-phenylamino}-imidazo[1,2-a]pyrazin-6-yl)-phenyl]-benzamide;
4-tert-Butyl-N-{3-[8-(4-carbamoylmethyl-phenylamino)-imidazo[1,2-a]pyrazin-6-yl]-phenyl}-benzamide;
3-(4-{6-[3-(4-tert-Butyl-benzoylamino)-phenyl]-imidazo[1,2-a]pyrazin-8-ylamino}-phenyl)-propionic acid;
4-tert-Butyl-N-(3-{8-[4-(2-hydroxy-ethyl)-phenylamino]-imidazo[1,2-a]pyrazin-6-yl}-phenyl)-benzamide;
4-tert-Butyl-N-(3-{8-[4-(2-methylamino-ethyl)-phenylamino]-imidazo[1,2-a]pyrazin-6-yl}-phenyl)-benzamide;
4-tert-Butyl-N-{3-[8-(4-methylaminomethyl-phenylamino)-imidazo[1,2-a]pyrazin-6-yl]-phenyl}-benzamide;
4-{6-[3-(4-tert-Butyl-benzoylamino)-phenyl]-imidazo[1,2-a]pyrazin-8-ylamino}-2-hydroxy-benzoic acid;
4-tert-Butyl-N-(3-{8-[4-(1-hydroxy-1-methyl-ethyl)-phenylamino]-imidazo[1,2-a]pyrazin-6-yl}-phenyl)-benzamide;
4-tert-Butyl-N-(3-{8-[4-(2-hydroxy-ethoxy)-phenylamino]-imidazo[1,2-a]pyrazin-6-yl}-phenyl)-benzamide;
4-tert-Butyl-N-(3-{8-[4-(4-methyl-piperazine-1-carbonyl)-phenylamino]-imidazo[1,2-a]pyrazin-6-yl}-phenyl)-benzamide;
4-tert-Butyl-N-(3-{8-[4-(2-methylamino-ethoxy)-phenylamino]-imidazo[1,2-a]pyrazin-6-yl}-phenyl)-benzamide;
4-tert-Butyl-N-(3-{8-[4-(2-dimethylamino-ethoxy)-phenylamino]-imidazo[1,2-a]pyrazin-6-yl}-phenyl)-benzamide;
4-{6-[3-(4-tert-Butyl-benzoylamino)-phenyl]-imidazo[1,2-a]pyrazin-8-ylamino}-benzoic methoxy amide;
4-{6-[3-(4-tert-Butyl-benzoylamino)-phenyl]-imidazo[1,2-a]pyrazin-8-ylamino}-benzoic hyrdoxyl methyl amide;
4-tert-Butyl-N-(3-{8-[4-([1,4]diazepane-1-carbonyl)-phenylamino]-imidazo[1,2-a]pyrazin-6-yl}-phenyl)-benzamide;
4-tert-Butyl-N-(3-{8-[4-(pyrrolidine-1-carbonyl)-phenylamino]-imidazo[1,2-a]pyrazin-6-yl}-phenyl)-benzamide;
4-tert-Butyl-N-(3-{8-[4-(morpholine-4-carbonyl)-phenylamino]-imidazo[1,2-a]pyrazin-6-yl}-phenyl)-benzamide;
4-tert-Butyl-N-(3-{8-[4-(ethoxyamine-4-carbonyl)-phenylamino]-imidazo[1,2-a]pyrazin-6-yl}-phenyl)-benzamide;
4-tert-Butyl-N-(3-{8-[4-(dimethylamine-4-carbonyl)-phenylamino]-imidazo[1,2-a]pyrazin-6-yl}-phenyl)-benzamide;
4-tert-Butyl-N-(3-{8-[4-(methylethylamine-4-carbonyl)-phenylamino]-imidazo[1,2-a]pyrazin-6-yl}-phenyl)-benzamide;
4-tert-Butyl-N-(3-{8-[4-(3-oxo-piperazine-1-carbonyl)-phenylamino]-imidazo[1,2-a]pyrazin-6-yl}-phenyl)-benzamide;
4-tert-Butyl-N-(3-{8-[N,N,N′-Trimethyl-ethane-1,2-diamine-4-carbonyl)-phenylamino]-imidazo[1,2-a]pyrazin-6-yl}-phenyl)-benzamide;
4-tert-Butyl-N-(3-{8-[4-(4-methyl-[1,4]diazepane-1-carbonyl)-phenylamino]-imidazo[1,2-a]pyrazin-6-yl}-phenyl)-benzamide;
4-tert-Butyl-N-(3-{8-[4-(N-methyl ethoxyamine-4-carbonyl)-phenylamino]-imidazo[1,2-a]pyrazin-6-yl}-phenyl)-benzamide;
4-tert-Butyl-N-(3-{8-[4-(4-ethyl-piperazine-1-carbonyl)-phenylamino]-imidazo[1,2-a]pyrazin-6-yl}-phenyl)-benzamide;
4-{6-[3-(4-tert-Butyl-benzoylamino)-phenyl]-imidazo[1,2-a]pyrazin-8-ylamino}-benzoic benzyl amide;
4-tert-Butyl-N-(3-{8-[4-(4-methyl-piperazin-1-ylmethyl)-phenylamino]-imidazo[1,2-a]pyrazin-6-yl}-phenyl)-benzamide;
4-tert-Butyl-N-(3-{8-[4-(4-ethyl-piperazin-1-ylmethyl)-phenylamino]-imidazo[1,2-a]pyrazin-6-yl}-phenyl)-benzamide;
4-tert-Butyl-N-{3-[8-(4-[1,4]diazepan-1-ylmethyl-phenylamino)-imidazo[1,2-a]pyrazin-6-yl]-phenyl}-benzamide;
4-tert-Butyl-N-(3-{8-[4-(3-methyl-piperazin-1-ylmethyl)-phenylamino]-imidazo[1,2-a]pyrazin-6-yl}-phenyl)-benzamide;
4-tert-Butyl-N-{3-[8-(4-dimethylaminomethyl-phenylamino)-imidazo[1,2-a]pyrazin-6-yl]-phenyl}-benzamide;
4-tert-Butyl-N-[3-(8-{4-[(ethyl-methyl-amino)-methyl]-phenylamino}-imidazo[1,2-a]pyrazin-6-yl)-phenyl]-benzamide;
4-tert-Butyl-N-{3-[8-(4-{[(2-methoxy-ethyl)-methyl-amino]-methyl}-phenylamino)-imidazo[1,2-a]pyrazin-6-yl]-phenyl}-benzamide;
4-tert-Butyl-N-[3-(8-{4-[(2-methoxy-ethylamino)-methyl]-phenylamino}-imidazo[1,2-a]pyrazin-6-yl)-phenyl]-benzamide;
4-tert-Butyl-N-{3-[8-(4-{[(2-dimethylamino-ethyl)-methyl-amino]-methyl}-phenylamino)-imidazo[1,2-a]pyrazin-6-yl]-phenyl}-benzamide;
N-(3-{8-[4-(4-Acetyl-piperazine-1-carbonyl)-phenylamino]-imidazo[1,2-a]pyrazin-6-yl}-phenyl)-4-tert-butyl-benzamide;
4-tert-Butyl-N-{3-[8-(4-{[(2-hydroxy-ethyl)-methyl-amino]-methyl}-phenylamino)-imidazo[1,2-a]pyrazin-6-yl]-phenyl}-benzamide;
4-tert-Butyl-N-[3-(8-{4-[(2-hydroxy-ethylamino)-methyl]-phenylamino}-imidazo[1,2-a]pyrazin-6-yl)-phenyl]-benzamide;
4-tert-Butyl-N-(3-{8-[4-(4-methyl-[1,4]diazepan-1-ylmethyl)-phenylamino]-imidazo[1,2-a]pyrazin-6-yl}-phenyl)-benzamide;
N-[3-(8-{4-[2-(Acetyl-methyl-amino)-ethoxy]-phenylamino}-imidazo[1,2-a]pyrazin-6-yl)-phenyl]-4-tert-butyl-benzamide;
4-(4-{6-[3-(4-tert-Butyl-benzoylamino)-phenyl]-imidazo[1,2-a]pyrazin-8-ylamino}-phenyl)-piperazine-1-carboxylic acid ethyl ester;
N-(3-{8-[4-(4-Acetyl-piperazin-1-ylmethyl)-phenylamino]-imidazo[1,2-a]pyrazin-6-yl}-phenyl)-4-tert-butyl-benzamide;
4-tert-Butyl-N-{3-[8-(4-imidazol-1-ylmethyl-phenylamino)-imidazo[1,2-a]pyrazin-6-yl]-phenyl}-benzamide;
4-tert-Butyl-N-{3-[8-(4-pyrrolidin-1-ylmethyl-phenylamino)-imidazo[1,2-a]pyrazin-6-yl]-phenyl}-benzamide;
N-[3-(8-{4-[(Acetyl-methyl-amino)-methyl]-phenylamino}-imidazo[1,2-a]pyrazin-6-yl)-phenyl]-4-tert-butyl-benzamide;
4-tert-Butyl-N-[3-(8-{4-[(methanesulfonyl-methyl-amino)-methyl]-phenylamino}-imidazo[1,2-a]pyrazin-6-yl)-phenyl]-benzamide;
4-tert-Butyl-N-(3-{8-[4-(3-isopropyl-1-methyl-ureidomethyl)-phenylamino]-imidazo[1,2-a]pyrazin-6-yl}-phenyl)-benzamide;
4-Isopropyl-N-(3-{8-[4-(morpholine-4-carbonyl)-phenylamino]-imidazo[1,2-a]pyrazin-6-yl}-phenyl)-benzamide;
N-{3-[8-(4-{[(2-Hydroxy-ethyl)-methyl-amino]-carbonyl}-phenylamino)-imidazo[1,2-a]pyrazin-6-yl]-phenyl}-4-isopropyl-benzamide;
N-(3-{8-[4-(Morpholine-4-carbonyl)-phenylamino]-imidazo[1,2-a]pyrazin-6-yl}-phenyl)-benzamide;
4-Methyl-N-(3-{8-[4-(morpholine-4-carbonyl)-phenylamino]-imidazo[1,2-a]pyrazin-6-yl}-phenyl)-benzamide;
4-Ethyl-N-(3-{8-[4-(morpholine-4-carbonyl)-phenylamino]-imidazo[1,2-a]pyrazin-6-yl}-phenyl)-benzamide;
4-Fluoro-N-(3-{8-[4-(morpholine-4-carbonyl)-phenylamino]-imidazo[1,2-a]pyrazin-6-yl}-phenyl)-benzamide;
N-(3-{8-[4-(Morpholine-4-carbonyl)-phenylamino]-imidazo[1,2-a]pyrazin-6-yl}-phenyl)-4-trifluoromethyl-benzamide;
4-tert-Butyl-N-(3-{8-[4-(5,6-dihydro-8H-imidazo[1,2-a]pyrazine-7-carbonyl)-phenylamino]-imidazo[1,2-a]pyrazin-6-yl}-phenyl)-benzamide;
4-tert-Butyl-N-(3-{8-[4-(3-oxo-piperazin-1-ylmethyl)-phenylamino]-imidazo[1,2-a]pyrazin-6-yl}-phenyl)-benzamide;
1-(4-{6-[3-(4-tert-Butyl-benzoylamino)-phenyl]-imidazo[1,2-a]pyrazin-8-ylamino}-benzyl)-pyrrolidine-2-carboxylic acid methyl ester;
N-(3-{8-[4-(Morpholine-4-carbonyl)-phenylamino]-imidazo[1,2-a]pyrazin-6-yl}-phenyl)-terephthalamic acid methyl ester;
N-(3-{8-[4-(Morpholine-4-carbonyl)-phenylamino]-imidazo[1,2-a]pyrazin-6-yl}-phenyl)-terephthalamic acid;
N-(3-{8-[4-(4-Acetyl-piperazin-1-yl)-phenylamino]-imidazo[1,2-a]pyrazin-6-yl}-phenyl)-4-ethyl-benzamide;
N-(3-{8-[4-(4-Acetyl-piperazin-1-yl)-phenylamino]-imidazo[1,2-a]pyrazin-6-yl}-phenyl)-4-trifluoromethyl-benzamide;
4-tert-Butyl-N-(3-{8-[4-(3-methyl-5,6-dihydro-8H-[1,2,4]triazolo[4,3-a]pyrazin-7-ylmethyl)-phenylamino]-imidazo[1,2-a]pyrazin-6-yl}-phenyl)-benzamide;
4-tert-Butyl-N-(3-{8-[4-(3-methyl-5,6-dihydro-8H-[1,2,4]triazolo[4,3-a]pyrazine-7-carbonyl)-phenylamino]-imidazo[1,2-a]pyrazin-6-yl}-phenyl)-benzamide;
N-(3-{8-[4-(4-Acetyl-piperazin-1-yl)-phenylamino]-imidazo[1,2-a]pyrazin-6-yl}-phenyl)-4-isopropyl-benzamide;
N-(3-{8-[4-(4-Acetyl-piperazine-1-carbonyl)-phenylamino]-imidazo[1,2-a]pyrazin-6-yl}-phenyl)-4-isopropyl-benzamide;
1-(4-{6-[3-(4-tert-Butyl-benzoylamino)-phenyl]-imidazo[1,2-a]pyrazin-8-ylamino}-benzyl)-pyrrolidine-2-carboxylic acid;
4-Isopropyl-N-(3-{8-[4-(3-methyl-5,6-dihydro-8H-[1,2,4]triazolo[4,3-a]pyrazine-7-carbonyl)-phenylamino]-imidazo[1,2-a]pyrazin-6-yl}-phenyl)-benzamide;
N-(3-{8-[4-(5,6-Dihydro-8H-imidazo[1,2-a]pyrazine-7-carbonyl)-phenylamino]-imidazo[1,2-a]pyrazin-6-yl}-phenyl)-4-isopropyl-benzamide;
4-Isopropyl-N-(3-{8-[4-(3-oxo-piperazine-1-carbonyl)-phenyl amino]-imidazo[1,2-a]pyrazin-6-yl}-phenyl)-benzamide;
4-Isopropyl-N-(3-{8-[4-(4-methyl-piperazine-1-carbonyl)-phenylamino]-imidazo[1,2-a]pyrazin-6-yl}-phenyl)-benzamide;
4-Isopropyl-N-(3-{8-[4-(3-oxo-piperazin-1-ylmethyl)-phenylamino]-imidazo[1,2-a]pyrazin-6-yl}-phenyl)-benzamide;
N-(3-{8-[4-(4-Acetyl-piperazin-1-ylmethyl)-phenylamino]-imidazo[1,2-a]pyrazin-6-yl}-phenyl)-4-isopropyl-benzamide;
N-(3-{8-[4-(Morpholine-4-carbonyl)-phenylamino]-imidazo[1,2-a]pyrazin-6-yl}-phenyl)-isonicotinamide;
N-(3-{8-[4-(Morpholine-4-carbonyl)-phenylamino]-imidazo[1,2-a]pyrazin-6-yl}-phenyl)-nicotinamide;
4-tert-Butyl-N-(3-{8-[4-(2-morpholin-4-yl-2-oxo-ethyl)-phenylamino]-imidazo[1,2-a]pyrazin-6-yl}-phenyl)-benzamide;
4-tert-Butyl-N-(3-{8-[3-(2-morpholin-4-yl-2-oxo-ethyl)-phenylamino]-imidazo[1,2-a]pyrazin-6-yl}-phenyl)-benzamide;
4-Isopropyl-N-{3-[8-(4-morpholin-4-ylmethyl-phenylamino)-imidazo[1,2-a]pyrazin-6-yl]-phenyl}-benzamide;
4-tert-Butyl-N-{3-[8-(4-morpholin-4-ylmethyl-phenylamino)-imidazo[1,2-a]pyrazin-6-yl]-phenyl}-benzamide;
N-(3-{8-[4-(Morpholine-4-carbonyl)-phenylamino]-imidazo[1,2-a]pyrazin-6-yl}-phenyl)-2-phenyl-acetamide;
N-(4-tert-Butyl-phenyl)-3-{8-[4-(morpholine-4-carbonyl)-phenylamino]-imidazo[1,2-a]pyrazin-6-yl}-benzamide;
N-(3-Chloro-benzyl)-3-{8-[4-(morpholine-4-carbonyl)-phenylamino]-imidazo[1,2-a]pyrazin-6-yl}-benzamide;
4-Cyano-N-(3-{8-[4-(morpholine-4-carbonyl)-phenylamino]-imidazo[1,2-a]pyrazin-6-yl}-phenyl)-benzamide;
6-Dimethylamino-N-(3-{8-[4-(morpholine-4-carbonyl)-phenylamino]-imidazo[1,2-a]pyrazin-6-yl}-phenyl)-nicotinamide;
N-Methyl-N′-(3-{8-[4-(morpholine-4-carbonyl)-phenylamino]-imidazo[1,2-a]pyrazin-6-yl}-phenyl)-terephthalamide;
N,N-Dimethyl-N′-(3-{8-[4-(morpholine-4-carbonyl)-phenylamino]-imidazo[1,2-a]pyrazin-6-yl}-phenyl)-terephthalamide;
4-Acetyl-N-(3-{8-[4-(morpholine-4-carbonyl)-phenylamino]-imidazo[1,2-a]pyrazin-6-yl}-phenyl)-benzamide;
4-(1H-Imidazol-2-yl)-N-(3-{8-[4-(morpholine-4-carbonyl)-phenylamino]-imidazo[1,2-a]pyrazin-6-yl}-phenyl)-benzamide;
2-(3-Isopropyl-phenoxy)-N-(3-{8-[4-(morpholine-4-carbonyl)-phenylamino]-imidazo[1,2-a]pyrazin-6-yl}-phenyl)-acetamide;
6-tert-Butyl-N-(3-{8-[4-(morpholine-4-carbonyl)-phenylamino]-imidazo[1,2-a]pyrazin-6-yl}-phenyl)-nicotinamide;
4-(6-{3-[(6-tert-Butyl-pyridine-3-carbonyl)-amino]-phenyl}-imidazo[1,2a]pyrazin-8-ylamino)-benzoic acid;
4-{6-[5-(4-tert-Butyl-benzoylamino)-pyridin-3-yl]-imidazo[1,2-a]pyrazin-8-ylamino}-benzoic acid;
4-tert-Butyl-N-(3-{8-[4-(octahydro-pyrido[1,2-a]pyrazine-2-carbonyl)-phenylamino]-imidazo[1,2-a]pyrazin-6-yl}-phenyl)-benzamide;
4-tert-Butyl-N-[3-(8-{4-[1-methyl-2-(octahydro-pyrido[1,2-a]pyrazin-2-yl)-2-oxo-ethyl]-phenylamino}-imidazo[1,2-a]pyrazin-6-yl)-phenyl]-benzamide;
Thiazole-4-carboxylic acid (3-{8-[4-(morpholine-4-carbonyl)-phenylamino]-imidazo[1,2a]pyrazin-6-yl}-phenyl)-amide;
[1,2,3]Thiadiazole-4-carboxylic acid (3-{8-[4-(morpholine-4-carbonyl)-phenylamino]-imidazo[1,2-a]pyrazin-6-yl}-phenyl)-amide;
Isoxazole-5-carboxylic acid (3-{8-[4-(morpholine-4-carbonyl)-phenylamino]-imidazo[1,2-a]pyrazin-6-yl}-phenyl)-amide;
6-Chloro-N-(3-{8-[4-(morpholine-4-carbonyl)-phenylamino]-imidazo[1,2-a]pyrazin-6-yl}-phenyl)-nicotinamide;
4-Hydroxy-6-methyl-N-(3-{8-[4-(morpholine-4-carbonyl)-phenylamino]-imidazo[1,2-a]pyrazin-6-yl}-phenyl)-nicotinamide;
and pharmaceutically acceptable salts, solvates, crystal forms, chelates, non-covalent complexes, prodrugs, and mixtures thereof.
